(19) United States
(12) Patent Application Publication
(10) Pub. No.: US 2026/0091147 A1
(43) Pub. Date: Apr. 2, 2026

(54) METHOD FOR DETECTING INNATE IMMUNE ACTION IN VIVO USING GPR84-PET

(71) Applicant: The Board of Trustees of the Leland Standford Junior University, Stanford, CA (US)

(72) Inventors: Michelle L. James, Palo Alto, CA (US); Isaac M. Jackson, San Bruno, CA (US); Mausam Kalita, Belmont, CA (US); Sydney C. Nagy, Portland, OR (US)

(21) Appl. No.: 19/475,452

(22) PCT Filed: Apr. 17, 2024
(86) PCT No.: PCT/US2024/024901

§ 371 Date: Oct. 15, 2025

Related U.S. Application Data
(60) Provisional application No. 63/496,431, filed on Apr. 17, 2023.

(51) Int. Cl. A61K 51/04; A61B 6/03; C07D 471/04; C07D 519/00

(52) U.S. Cl. CPC A61K 51/0459; A61B 6/037; C07D 471/04; C07D 519/00; A61K 2123/00

(57) ABSTRACT

In one aspect, the disclosure relates to ¹¹C and ¹⁸F radiolabeled compounds useful as positron emission tomography (PET) tracers for selective imaging of neuroinflammation associated with expression transmembrane protein GPR84, methods of making the same, and compositions comprising the same. In one aspect, the compounds can cross the blood brain barrier, have a high signal to noise ratio, a large dynamic range, and are highly specific for their targets. This abstract is intended as a scanning tool for purposes of searching in the particular art and is not intended to be limiting of the present disclosure.

### CROSS REFERENCE TO RELATED APPLICATIONS

This application claims the benefit of U.S. Provisional Application No. 63/496,431 filed on Apr. 17, 2023, which is incorporated herein by reference in its entirety.

### BACKGROUND

Limitations of current positron emission tomography (PET) approaches for imaging neuroinflammation necessitate development of improved PET biomarkers and tracers. Unfortunately, a lack of effective methods for predicting central nervous system (CNS) uptake of tracers prior to in vivo testing means that novel tracer development remains an extremely resource intensive process. While computational approaches, particularly machine learning (ML), have greatly enhanced drug development, key differences between drugs and PET tracers preclude direct application to CNS tracer design.

Maladaptive innate immune activation is a key driver and it, along with neuroinflammation, are hallmarks of many central nervous system (CNS) diseases including Alzheimer's disease, multiple sclerosis, glioblastoma, and stroke. Neuroinflammation is also an underlying feature of CNS trauma or injury and psychiatric disorders. Diagnostic approaches for CNS disease, including neurological and psychological exams, genetic tests, structural imaging (e.g. CT or MRI), testing of blood and cerebrospinal fluid (CSF), and postmortem analyses lack sensitivity and specificity.

Molecular imaging techniques such as PET can enable non-invasive visualization of biochemical processes longitudinally in living subjects. Unfortunately, most previously investigated biomarkers for quantifying neuroinflammation in vivo using positron emission tomography (PET) imaging have several limitations. For example, the most widely evaluated PET tracers for neuroinflammation target the translocator protein 18 kDa (TSPO), which is expressed indiscriminately on activated myeloid lineage cells, reactive astrocytes, and endothelial cells. TSPO PET tracers also suffer from variable binding affinity in human subjects due to a polymorphism in the TSPO gene. Other currently used technologies include PET tracers such as ¹⁸F-OP-801, which is a dendrimer-based PET tracer. This dendrimer agent is large in size and, thus, cannot cross the intact blood brain barrier.

In contrast, GPR84 is a seven transmembrane domain orphan G-protein coupled receptor (GPCR) expressed predominantly on pro-inflammatory myeloid cells (including macrophages, microglia, and neutrophils) following CNS injury or insult. Importantly, the expression of GPR84 is significantly upregulated on these innate immune cells after exposure to inflammatory stimuli (FIGS. 1A-1B) such as lipopolysaccharide (LPS) and tumor necrosis factor (TNF)-α. In murine tissues, GPR84 has been shown to be elevated by endotoxemia, hyperglycemia, and hypercholesterolemia. Likewise, LPS has been shown to dramatically increase expression of GPR84 mRNA in human monocyte-derived macrophages (hMDMs). Functionally, GPR84 has been shown to mediate the release of TNF-α and interleukin (IL)-12 from macrophages, as well as reduce cyclic adenosine monophosphate (cAMP) production, collectively enhancing and propagating pro-inflammatory signaling pathways. Moreover, there is growing evidence highlighting the involvement of GPR84 in many inflammatory, metabolic, and neurological conditions including IBD, atherosclerosis, pulmonary fibrosis, neuropathic pain, stroke, and Alzheimer's disease. The selective elevation in expression of GPR84 on innate immune cells, combined with its mechanistic association with amplifying inflammation in several diseases of interest, motivated pursuit of the development of PET radiotracers for this promising biomarker.

LPS (1 mg/kg) elevates GPR84 mRNA expression in mouse brain, lung, adipose tissue, and bone marrow, with microglial GPR84 messenger RNA (mRNA) increasing 4-fold versus its basal expression. This effect is absent in GPR84^{-/-} cells or following GPR84 antagonist treatment as AKT and ERK phosphorylation is partially blocked and expression of proinflammatory cytokines (interleukin [IL]-6, IL-12b, and tumor necrosis factor [TNF]-α) thus reduced. Audoy-Rémus et al. reported GPR84 upregulation in microglia of APP/PS1 transgenic mice model of AD. The GPR84 senses an elusive ligand that recruits microglia to the amyloid plaques. Microglial GPR84 expression is also elevated in experimental autoimmune encephalomyelitis (EAE), a mouse model of multiple sclerosis; mice suffering from endotoxemia also produced significant amount of GPR84 in microglia, and, to a lesser extent, subpopulations of peripheral macrophages and monocytes. Pro-inflammatory cytokines (tumor necrosis factor [TNF]-α and interleukin [IL]-1) likely regulate the endotoxin-induced GPR84 expression in the cerebral cortex of mouse. In the mouse model of neuropathic pain, microglial GPR84 interacts with the adaptor molecule downstream of kinase-3 (DOK3), thus provoking the inflammatory response. Hyperglycemia and hypercholesterolemia also stimulate GPR84 expression in murine tissues.

Several GPR84 antagonists have been developed for potential therapeutic use. However, many therapeutic molecules are not suitable for use as PET tracers and prediction of effectiveness of a molecule as a CNS penetrant PET tracer is not straightforward and, thus, GPR84 PET tracers remain undeveloped. What is needed is generation of PET tracers that target only activated myeloid cells, key innate immune effector cells. Such tracers would enable high fidelity monitoring of subtle changes in the spatiotemporal dynamics and phenotypes of myeloid cells in different disease types and stages.

Despite advances in neuroimaging research, there is still a scarcity of compounds that are effective GPR84 PET tracers useful for selective imaging of neuroinflammation, such as, for example on pro-inflammatory myeloid cells. Such tracers would ideally be able to cross the blood brain barrier and allow interrogation of innate immune activation in all contexts. They would further have a high signal to noise ratio, a large dynamic range, be highly specific for their targets, and would have functional relevance. These needs and other needs are satisfied by the present disclosure.

### SUMMARY

In accordance with the purpose(s) of the present disclosure, as embodied and broadly described herein, the disclosure, in one aspect, relates to ¹¹C and ¹⁸F radiolabeled compounds useful as positron emission tomography (PET) tracers for selective imaging of neuroinflammation associated with expression transmembrane protein GPR84, methods of making the same, and compositions comprising the same. In one aspect, the compounds can cross the blood brain barrier, have a high signal to noise ratio, a large dynamic range, and are highly specific for their target.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1B show using human brain RNA-seq, mouse brain analysis, and cellular analysis in human monocyte derived macrophages that GPR84 is a more sensitive and specific biomarker of microglia/macrophages than TSPO.

FIG. 2A shows CNSPOP and a support vector ML algorithm can differentiate permeable from non-permeable CNS PET tracers with good sensitivity, PPV, and overall accuracy. CNSPOP score and key physicochemical parameters of GLGP38 are encouraging for CNS uptake. FIG. 2B shows dynamic PET imaging of a Rhesus macaque was conducted over 60 minutes and the resulting time activity curve shows significant CNS uptake (3.3 SUV at 4 minutes post injection). FIGS. 2C-2D show these analyses for a poorly performing tracer ([¹¹C]AZD1283) for comparative purposes.

FIGS. 3A-3C show synthesis and characterization for [¹¹C]GLPG38. FIG. 3A shows an exemplary synthesis scheme, FIG. 3B shows co-elution of [¹¹C]GLPG38 with a no-radioactive standard, and FIG. 3C shows a competitive binding assay with a known GPR84 inhibitor.

FIGS. 4A-4B show cell binding experiments were performed first with hGPR84-stably-transduced human embryonic kidney cells (HEK-293) versus control HEK-293 cells. To assess binding specificity, cells were incubated with the tracer in absence or presence of a blocking compound, the known GPR84 antagonist (35 μM GLPG1205 in DMSO and media), for 40 mins. Cell binding studies showed 13.5-fold higher binding of [¹¹C]GLPG38 to hGPR84-HEK-293 cells vs control cells (p<0.0001, n=4). Co-incubation with GPR84 antagonist (GLPG1205, 1000× by mass) reduced tracer binding in hGPR84-HEK293 cells by >90% (p<0.0001, n=4) proving high specificity of these tracers in cells. Notably, hMDMs challenged with LPS (100 ng/mL) stimulus were found to display significantly higher binding of [¹¹C]GLPG38 compared to unstimulated cells (p>0.001, n=3), and this signal was reduced to baseline following co-incubation with GLPG1205 (p>0.001, n=3). Statistical significance was assessed using a one-way ANOVA: ***p<0.001, ****p<0.0001, ns p>0.05.

FIG. 5 shows time activity curves depicting in vivo uptake of [¹¹C]GLPG38 in whole mouse brain (n=4) over the course of 60 min dynamic PET/CT imaging.

FIGS. 6A-6C show biodistribution results (% ID/g) of [¹¹C]GLPG38 for blood, left brain hemisphere, heart, lung, median lobe of the liver, kidney, spleen, posterior neck adipose tissue, and muscle of the thigh are shown following perfusion with PBS to remove blood from organs. Significant differences between LPS-treated and vehicle-treated animals were found in the brain (p=0.0023) and adipose tissue (p=0.007) with a trend in the spleen (p=0.0571) using Welch's t test: **p>0.01.

FIG. 7 shows representative images of 60 μm thick sagittal right hemibrain sections of mouse brain treated with LPS (left), vehicle (middle), or LPS+GLPG1205 blocking agent (right) show radioactive intensity (top), anatomical stain using cresyl violet Nissl staining (middle), and overlay of the radioactivity signal and anatomical staining (bottom). A marked increase in signal is observed in LPS-treated compared to vehicle or LPS+block-treated animals.

FIG. 8 shows in vitro ARG of [¹¹C]GLPG38 in active UC colon tissue samples demonstrated marked increase of binding for both tracers compared to age-, sex-, and ethnicity-matched healthy samples, and this signal was markedly attenuated by pre-blocking with 1000×GLPG1205 by mass. Regions of high tracer binding corresponded with immune infiltrates shown by H&E staining.

FIGS. 9A-9B show HPLC radiometabolite analysis to determine in vivo stability of ¹¹C-MGX-10S. Radiotracer stability was assessed in mouse (FIG. 9A) brain and (FIG. 9B) liver at 5 min (top panel) and 20 min (bottom panel) post injection. 5 min postinjection HPLC analysis of the brain and liver samples showed a major radioactive parental peak (HPLC elution time=5.0 min). 20 minutes postinjection HPLC analysis revealed a minor radiometabolite peak (M1 metabolite HPLC elution time=2.8 min): 3% in brain and 10% in liver.

FIG. 10A shows time-activity curves (TACs) of the whole brain from 60 min dynamic PET scan clearly shows rapid uptake of the radiotracer in the brain within 1 min of tracer injection followed by clearance. The radiotracer accumulated in the LPS brains 42.46% (at the end of the scan, P<0.05) more than saline-treated brain. GLPG1205 (0.65 mg/kg) reduced this accumulation by 38.50% to the saline baseline at the end of the scan. (FIG. 10B) ¹¹C-MGX-10S detected increased severity of innate immune responses and enabled neuroinflammation measurement (summed 30-60 min) as seen in the axial, coronal, and sagittal planes. (FIG. 10C) Dose-dependent blocking of GPR84 with GLPG1205 determined the radiotracer specificity in the brain. Liv=liver.

FIGS. 11A-11B show ex vivo biodistribution and autoradiography. (FIG. 11A) ¹¹C-MGX-10S radiotracer was administered in saline (n=5)- and LPS (5 mg/kg, n=5)-treated mice. At 20 min postinjection, animals were perfused with saline and tissues were collected, weighed, and gamma-counted. The LPS-induced systemic inflammation activated the innate immune response. The radiotracer accumulated in all organs, with the highest uptake in the liver and kidney. LPS mouse brains (0.84±0.21% ID/gram) displayed ˜1.85-fold increase in radiotracer uptake compared to saline (0.45±0.06% ID/g), suggesting overexpression of GPR84 on microglia. Statistical analysis was performed using “Unpaired t test with Welch correction”, *=P<0.05. (FIG. 11B) Ex vivo autoradiography and histology validation of the ¹¹C-MGX-10S radiotracer. This study highlights the specificity of the disclosed radiotracer in the brain. The LPS (5 mg/kg) brain retained marked radioactivity 20 min post injection compared to saline. The GLPG1205 antagonist blocked the radiotracer signal, suggesting GPR84 specific binding of ¹¹C-MGX-10S. Cb=cerebellum, Ctx=cortex, Str=striatum, H=hippocampus, Mb=midbrain, Ob=olfactory bulb, and BS=brain stem.

FIGS. 12A-12D show a comparison of GPR84 and TSPO PET radiotracers in a murine model of systemic innate immune activation. (FIG. 12A) In vivo PET imaging of TSPO via ¹¹C-DPA-713 enabled visualization of immune responses in the brain and peripheral organs in LPS-induced inflammation. Static PET images were acquired 50-60 min postinjection of ¹¹C-DPA-713. (FIG. 12B) Ex vivo biodistribution of ¹¹C-MGX-10S and ¹¹C-DPA-713 radiotracers revealed GPR84 and TSPO expressions respectively in brain, liver, lung, and spleen. Overall, ¹¹C-MGX-10S performed better than ¹¹C-DPA-713 in liver (P<0.01), lungs (P<0.05), and spleen (P<0.01)—known sites of activated immune cells and comparably in the brain and lung. (FIG. 12C) qPCR analysis of the tspo gene revealed no difference in its expression in LPS- and saline-treated mice (n=4, P=ns). (FIG. 12D) qPCR analysis of gpr84 gene expression demonstrated significant upregulation in brains from LPS (5 mg/kg)-induced systemic inflammation compared to saline-treated mice (n=4, P<0.0001). Statistical analysis was performed using “Unpaired t test with Welch correction”, *=P<0.05, **=P<0.01, ***=P<0.001, ****=P<0.0001.

FIGS. 13A-13C show radiosynthesis and cell binding for ¹¹C-MGX-10S and ¹¹C-MGX-11S. FIG. 13A shows a radiosynthetic scheme to generate ¹¹C-MGX-10S. FIG. 13B shows a radiosynthetic scheme to generate ¹¹C-MGX-11S. FIG. 13C shows in vitro tracer binding specificity (¹¹C-MGX-10S, left, and ¹¹C-MGX-11S, right) studies using human-GPR84-expressing HEK293 cells versus parental control cells (P=0.0001).

FIGS. 14A-14B show time activity curves depicting in vivo uptake of disclosed tracers and PET/CT images of the same over the course of 60 min dynamic PET/CT imaging. FIG. 14A shows time activity curves depicting in vivo uptake of ¹¹C-MGX-10S in whole mouse brain (n=4) over the course of 60 min dynamic PET/CT imaging. FIG. 14B shows time activity curves depicting in vivo uptake of ¹¹C-MGX-11S in whole mouse brain (n=4) over the course of 60 min dynamic PET/CT imaging.

FIGS. 15A-15B show HPLC chromatograms of the crude ¹¹C-MGX-10S and ¹¹C-MGX-11S tracers alone and their co-injection with cold standards.

FIGS. 16A-16C show competitive binding and functional assays of cold standards. FIG. 16A: Competition binding assay with the known GPR84 [³H]G9543 using membrane from Flp-In TREx 293 cells expressing the human GPR84-Gαᵢ₂ fusion protein (n=3). FIG. 16B: cAMP assay using Flp-In TREx 293 cells expressing the human GPR84-Gαᵢ₂ fusion protein (n=3). FIG. 16C: GTPγS assay: 3 mg of membranes from cells expressing the fusion human GPR84-Gi1/2 protein were used to pretreat compounds at increasing concentration and subsequently stimulated with a fixed concentration of the orthoseteric agonist 2-(hexylthiol)pyrimidine-4,6 diol (2-HTP).

FIG. 17 shows ex vivo biodistribution (BioD) of ¹¹C-MGX-10S (% ID/g) for blood, left brain hemisphere, heart, lung, median lobe of the liver, kidney, spleen, posterior neck adipose tissue, and muscle of the thigh are shown following perfusion with PBS to remove blood from organs. Significant differences between LPS-treated and vehicle-treated animals were found in the brain (p=0.0023) and adipose tissue (p=0.007) with a trend in the spleen (p=0.0571) using Welch's t test: **p>0.01.

FIGS. 18A-18B show binding assays of a series of GPR84 antagonists including GLPG38 to determine (FIG. 18A) K_{d} based on a competitive binding assay with radiolabeled GPR84 antagonist [³H]G9543 and (FIG. 18B) IC₅₀ via a cAMP inhibition assay.

FIG. 19 shows identity and radiochemical purity of [¹¹C]GLPG38 was confirmed via HPLC co-injection with reference standard.

FIG. 20 shows a cell binding study of [¹¹C]GLPG38 with hGPR84 expressing HEK293 cells to determine binding specificity. [¹¹C]GLPG38 demonstrated significantly higher uptake in hGPR84⁺HEK293 cells at 20 and 40 minutes compared to parental control cells. Uptake was significantly decreased in hGPR84⁺HEK293 cells in the presence of GPR84 antagonist GLPG1205 as a blocking agent.

FIGS. 21A-21B show qPCR comparison of TSPO vs GPR84 mRNA expression in brain between (FIG. 21A) naïve wild-type vs. mice treated with LPS as a murine model of sepsis and (FIG. 21B) wild-type vs. 5×FAD transgenic mice.

FIG. 22 shows a time-activity curve (TAC) showing PET signal in the whole brain of 4 naïve wild-type mice mouse over the duration of a 60 min dynamic scan.

FIGS. 23A-23B show HPLC characterization of ¹⁸F-MGX-110S. Pure ¹⁸F-MGX-110S (HPLC retention time=7.14 min, FIG. 23A) was confirmed by analytical HPLC using co-injection of the tracer with cold ¹⁹F-MGX-110S compound (HPLC retention time=7.02 min, FIG. 23B).

FIGS. 24A-24B show HPLC characterization of ¹⁸F-MGX-111S. Pure ¹⁸F-MGX-111S (HPLC retention time=8.06 min, FIG. 24A) was confirmed by analytical HPLC using co-injection of the tracer with cold ¹⁹F-MGX-110S compound (HPLC retention time=7.94 min, FIG. 24B).

FIG. 25 shows a competition binding experiment. 5 μg of membranes was prepared from a stable cell line expressing hGPR84-Gi fusion protein and measured the ability of the compounds to compete for binding of [³H]G9543 (chemical analog of lead negative allosteric modulator, GLPG1205). Log Kᵢ values are shown in the table.

FIG. 26A shows cell binding experiments were performed first with hGPR84-stably-transduced human embryonic kidney cells (HEK-293) versus control HEK-293 cells. To assess binding specificity, cells were incubated with the tracer in absence or presence of a blocking compound, the known GPR84 antagonist (35 μM GLPG1205 in DMSO and media), for 40 mins. Cell binding studies showed 25-fold higher binding of [¹⁸F]MGX-110S to hGPR84-HEK-293 cells vs control cells (p<0.0001, n=5-7). Co-incubation with GPR84 antagonist (GLPG1205, 1000× by mass) reduced tracer binding in hGPR84-HEK293 cells by >90% (p<0.0001, n=5-7) proving high specificity of these tracers in cells. FIG. 26B shows hMDMs challenged with LPS (100 ng/mL) stimulus were found to display significantly higher binding of [¹⁸F]MGX-110S compared to unstimulated cells (p>0.001, n=3), and this signal was reduced to baseline following co-incubation with GLPG1205 (p>0.001, n=3). Statistical significance was assessed using a one-way ANOVA: *p<0.05, **p<0.01, ****p<0.0001, ns p>0.05.

FIG. 27 shows a time activity curve illustrating kinetics of ¹⁸F-MGX-110S in the whole brain of a healthy naïve mouse demonstrating radiotracer crossing the blood brain barrier with over 6% ID/g peak uptake and a rapid washout with less than 1.5% ID/g at 60-min post-injection.

FIGS. 28A-28B show spinal cord time activity curves illustrating increased signal of ¹⁸F-MGX-110S in both the lumbar and cervical/thoracic spinal cord of EAE compared to naive mice. Significantly higher signal at 60 min in the lumbar was found with Welch's t test. *p<0.05, **p<0.01, ***p<0.001, ****p<0.0001, ns p>0.05.

FIG. 29A shows gamma counting of spinal cords confirmed elevated uptake of ¹⁸F-MGX-110S using Welch's t test. *p<0.05, **p<0.01, ***p<0.001, ****p<0.0001, ns p>0.05. FIG. 29B shows qPCR of an EAE lumbar spinal cord (“MS-like”) mouse model indicating that TSPO PET is less sensitive at detecting inflammation in this model. qPCR shows that GPR84 is significantly upregulated in the spinal cord of an MS mouse model and this elevation can be detected using the disclosed tracers with a 1.5-1.9 fold increase in signal.

FIG. 30A shows mRNA expression of Gpr84 in spinal cords from EAE versus naive mice demonstrating significantly higher Gpr84 expression in all segments of the spinal cord via ordinary one way ANOVA test. FIG. 30B shows a comparison using Welch's t test of naïve normalized Gpr84 vs Tspo mRNA in EAE spinal cord reveals significantly higher Gpr84 expression, indicating higher sensitivity of this biomarker in EAE mice. *p<0.05, **p<0.01, ***p<0.001, ****p<0.0001, ns p>0.05.

FIG. 31A shows representative high-resolution images of tracer binding in ex vivo autoradiography of lumbar spinal cord sections (40 μm-thick) shows tracer binding is highly specific for GPR84. FIG. 31B shows quantification of autoradiography data shows significantly more tracer binding in the high score EAE mouse compared to both the blocked high score and naïve groups using Welch's t test. #denotes comparison to naïve. *^{,#}p<0.05, **^{,##}p<0.01, ***^{,###}p<0.001, ****^{,####}p<0.0001, ns p>0.05.

FIGS. 32A-32E show post-scan ex vivo gamma counting confirms specificity of ¹⁸F-MGX-110S in all organs via significant blocking with GLPG1205, a known GPR84 agonist. Additionally, gamma counting shows significantly higher binding in high scoring EAE mice compared to Naïve mice in all organs except spleen and significantly higher radiotracer binding in the spinal cords of the low scoring EAE mice using Welch's t test. #denotes comparison to naïve. *^{,#}p<0.05, **^{,##}p<0.01, ***^{,###}p<0.001, ****^{,####}p<0.0001, ns p>0.05.

FIG. 33 shows ¹⁸F-MGX-110S can be used to monitor response to immunotherapy in a mouse model of brain metastasis by imaging GPR84 expressing immune cells in the tumor microenvironment. Six-to-eight-week-old female C57BL/6 mice were implanted subcutaneously with 0.25×10⁶ B16F1 melanoma cells on the right flank and with 5×10³ B16F1 melanoma cells in the striatum two days post-extracranial tumor inoculation. Mice were randomized into treatment and no treatment groups on day 5 post-intracranial tumor inoculation based on the intracranial bioluminescence signals. Mice in the treatment group were treated with anti-PD-1 and anti-CTLA-4 (200 μg via intraperitoneal injection) on days 5, 7 and 9. MRI was performed on day 9 post-intracranial tumor implantation to identify brain tumor of interest, and PET/CT imaging using ¹⁸F-MGX-110S followed. Representative PET/CT images of mouse with brain metastasis showed significantly elevated signal in the intracranial tumor compared to immunotherapy-treated mouse. ¹⁸F-MGX-110S can detect a significant reduction in signal representing GPR84-positive immune cells (e.g., myeloid derived suppressor cells; MDSCs) following treatment with anti-PD-1 and anti-CTLA-4 immunotherapies. Notably the signal (indicative of PET tracer binding) is very low in healthy brain and muscle which affords high signal-to-background images of innate immune cells in the tumor microenvironment of brain tumors (intracranial tumor) and also in the extracranial tumors. These data demonstrate the promise of ¹⁸F-MGX-110S for detecting and tracking immune cells with high specificity in the context of cancer. Importantly, GPR84 is not expressed on cancer cells or healthy brain tissue which is what enables imaging with ¹³F-MGX-110S to afford highly sensitive and specific imaging of immune cells in cancer before and after therapy.

FIG. 34 shows in vitro ARG of [¹⁸F]MGX-110S in active UC colon tissue samples demonstrated marked increase of binding for both tracers compared to age-, sex-, and ethnicity-matched healthy samples, and this signal was markedly attenuated by pre-blocking with 1000×GLPG1205 by mass. Regions of high tracer binding corresponded with immune infiltrates shown by H&E staining.

FIG. 35 shows quantification of PET images which demonstrates that ¹⁸F-MGX-110S can detect a significant reduction in signal representing GPR84-positive immune cells (e.g., myeloid derived suppressor cells; MDSCs) following treatment with anti-PD-1 and anti-CTLA-4 immunotherapies. Notably the signal (indicative of PET tracer binding) is very low in healthy brain and muscle which affords high signal-to-background images of innate immune cells in the tumor microenvironment of brain tumors (intracranial tumor) and also in the extracranial tumors. These data demonstrate the promise of ¹⁸F-MGX-110S for detecting and tracking immune cells with high specificity in the context of cancer. Importantly, GPR84 is not expressed on cancer cells or healthy brain tissue which is what enables imaging with ¹⁸F-MGX-110S to afford highly sensitive and specific imaging of immune cells in cancer before and after therapy.

FIGS. 36A-36C show post PET scan perfusion and dissection of CNS tissue followed by gamma counting confirms significantly decreased radiotracer binding in all CNS organs in a mouse model.

FIG. 37 shows using human brain RNA-seq, mouse brain analysis, and cellular analysis in human monocyte derived macrophages that GPR84 is a more sensitive and specific biomarker of microglia/macrophages than TSPO.

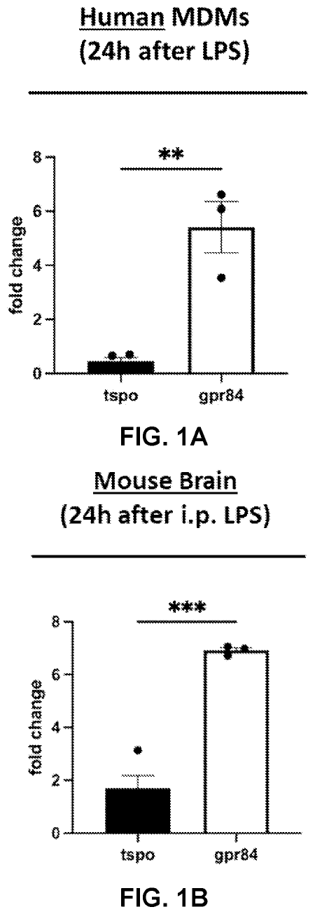

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Disclosed herein are new PET radiotracers labeled with ¹¹C and/or ¹⁸F for quantifying and tracking GPR84-positive cells in vivo. GPR84 PET tracers are far more specific for (and have the ability to detect subtle changes in) innate immune activation compared to existing methods (e.g., radiotracers that target TSPO, CSF1R, or P2×7). The disclosed PET radiotracers enable early and accurate diagnosis as well as real-time monitoring of therapeutic efficacy for neurological and other diseases with an inflammatory component through non-invasive molecular imaging of inflammation.

While GPR84 is an orphan receptor without a known endogenous ligand, [1,4]dioxan-2-ylmethoxy)-6,7-dihydropyrimido[6,1-a]isoquinolin-4-one (DDHPI) ligands were recently reported to be a potent and selective class of negative allosteric GPR84 modulators. The lead antagonist, GLPG1205, bound GPR84 with an inhibition constant of Kᵢ=7.52 nM and reduced disease activity score and human neutrophil infiltration (IC₅₀=11 nM) in a rodent model of inflammatory bowel disease. Importantly, GLPG1205 also showed positive results in a Phase II clinical trial of patients with idiopathic pulmonary fibrosis.

It is known in the art that effectiveness of a molecule as a PET tracer is not predictable based on use of the molecule as a therapeutic agent. In one aspect, the target property profile for tracers and the settings in which they are used can differ. Further in this aspect, small molecule tracers are used for imaging on the time scale of minutes to hours and are typically administered intravenously. In another aspect, when using a tracer for imaging, it is not desirable to see a therapeutic effect from the tracer. In a further aspect, tracers are typically administered in the nanomolar or subnanomolar ranges, so affinity of the tracer for the target rather than therapeutic effect is an important consideration. In another aspect, safety of the tracer at low doses and therapeutic efficacy of the tracer are less important relative to central nervous system uptake and specific binding to the target of interest. In some aspects, tracers can be designed without knowing the structures of clinical drug candidates. Further in this aspect, when a tracer is designed according to the approaches disclosed herein, clinical drug candidates are frequently not among the identified structures of tracer candidates.

Herein it is hypothesized that certain closely related structural analogs of GLPG1205 may be able to maintain selectivity for GPR84 and serve as high affinity PET tracers for assessing this new potential imaging biomarker in murine models of neuroinflammation.

In an aspect, disclosed herein is a radiolabeled compound having a structure of Formula I:

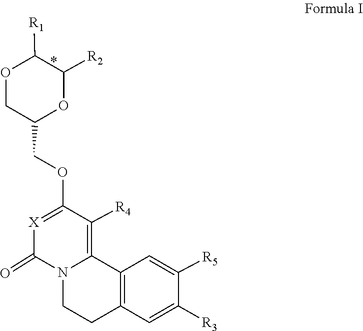

wherein X is N or CH;
wherein R₁ and R₂ are both hydrogen and the bond marked by * is a single bond, or wherein R₁ and R₂ are bridged to form a heteroaromatic ring and the bond marked by * is a component of the heteroaromatic ring;
wherein R₃ comprises a halogen, C1-C4 alkyl ether, or fluorosulfonate group;
   wherein R₃ comprises at least one radioisotope; and
wherein R₄ and R₅ are independently hydrogen or methyl.

In another aspect, the at least one radioisotope can be selected from ¹¹C and ¹⁸F. In a further aspect, R₁ and R₂ are both hydrogen and the bond marked by * is a single bond. In an alternative aspect, the radiolabeled compound has the structure:

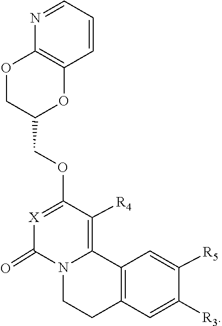

In some aspects, R₄ and R₅ are both methyl or are both hydrogen. In still another aspect, R₃ includes at least one ¹¹C atom or at least one ¹⁸F atom. In some aspects, R₃ can be selected from

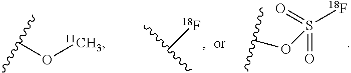

In one aspect, the radiolabeled compound is selected from

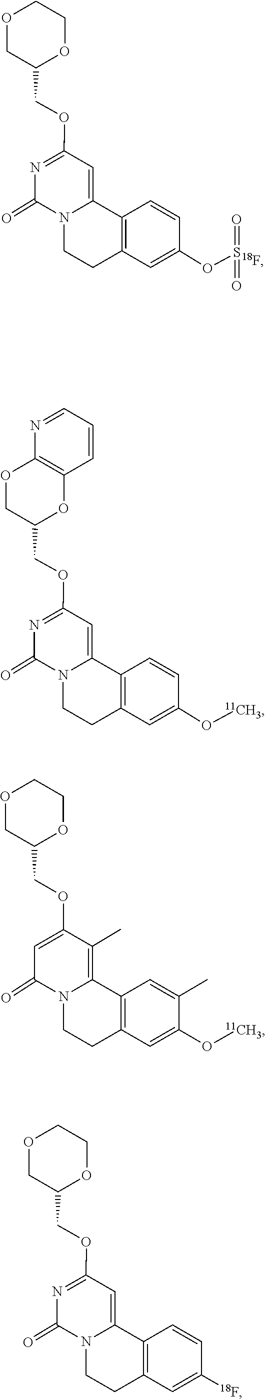

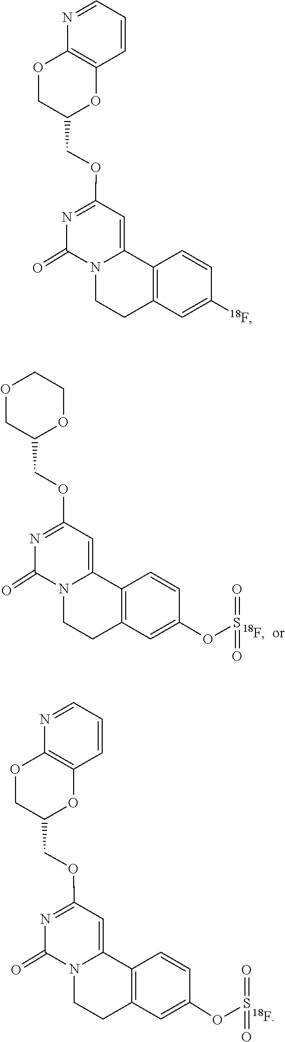

In any of these aspects, the radiolabeled compound can undergo beta plus decay and emit a positron. In some aspects, the radioisotope has a half life of at least about 20 minutes, or of at least about 100 minutes. Without wishing to be bound by theory, compounds including ¹⁸F can have a longer half life (e.g., about 100 minutes, about 105 minutes, about 110 minutes, or the like) than compounds including ¹¹C.

In any of these aspects, the radiolabeled compound is capable of penetrating an intact blood brain barrier (BBB).

Also disclosed herein are pharmaceutical compositions including the disclosed radiolabeled compounds. In one aspect, the compounds or pharmaceutical compositions containing the same can be administered to a patient by injection or intravenously. In a further aspect, the pharmaceutical compositions can further include one or more carriers or excipients to facilitate successful administration.

Also disclosed herein is a method for imaging neuroinflammation in a subject, the method including at least the steps of
(a) administering a disclosed radiolabeled compound or pharmaceutical composition to the subject; and
(b) performing positron emission tomography (PET) on the subject.

Further in this aspect, the inflammation can be associated with increased GPR84 expression relative to a level of GPR84 expression in a subject not experiencing inflammation. In one aspect, the radiolabeled compounds will not accumulate in a subject not experiencing inflammation, but will accumulate at the site of inflammation in a subject experiencing inflammation. In a further aspect, the inflammation can be neuroinflammation, gut inflammation, or any combination thereof. In one aspect, the radiolabeled compound binds to GPR84.

In another aspect, the subject is a mammal such as, for example, a human, mouse, rat, hamster, rabbit, guinea pig, dog, cat, horse, cattle, sheep, swine, or non-human primate.

Also disclosed herein is a method for determining a stage of disease in a subject, the method including at least the steps of
(a) administering the radiolabeled compound of any one of claims 1-16 or the pharmaceutical composition of claim 17 or 18 to the subject;
(b) performing a positron emission tomography (PET) scan on the subject; and
(c) comparing results of the PET scan to one or more standard PET scans.

In one aspect, the one or more standard PET scans can be of subjects diagnosed with specific stages of the disease. In a further aspect, the disease is associated with increased GPR84 expression. In yet another aspect, the disease can be selected from Alzheimer's disease, multiple sclerosis, glioblastoma, brain metastases, stroke, central nervous system (CNS) injury, a psychiatric disorder, multiple sclerosis, ulcerative colitis, another inflammatory bowel disease, atherosclerosis, pulmonary fibrosis, arthritis, neuropathic pain or other types of pain, chronic fatigue syndrome, fibromyalgia, a bacterial or viral infection, cancer, or any combination thereof.

In one aspect, the disclosed radiotracers, when administered to healthy patients, result in a negligible signal, indicating no active disease, while when administered to patients experiencing inflammation, result in a high signal indicative of inflammation.

In one aspect, inflammation is of central importance in diverse CNS and other pathologies. In a further aspect, microglia may play a significant role in the progression of lesions seen in diseases that are not primarily inflammatory such as, for example, Alzheimer's disease. In another aspect, in primary brain tumors, up to ⅓ of the cells in glioblastoma are microglia, while in epilepsy-related tumors, the density of microglia correlates with seizure duration and frequency. In a further aspect, in infections, much of the injury in brain infections is due to inflammation rather than the microorganisms associated with the infections, and microglia/macrophages have a role in viral diseases as well (e.g., HIV/AIDS and COVID). In still another aspect, in demyelinating and other autoimmune diseases, perivascular mononuclear cells such as lymphocytes, macrophages, and plasma cells are causative.

In one aspect, multiple sclerosis is primarily diagnosed in 20-30 year olds and affects about 2.8 million people worldwide. Early and accurate diagnosis can be difficult, with misdiagnosis being common. Disease-modifying treatments are available, but methods to select and monitor therapies are limited and involve some degree of inaccuracy. MRI is, at present, the gold standard for assessing MS-associated lesions but lacks sensitivity. Current PET radiotracers for neuroinflammation such as, for example, those that target translocator protein 18 kDa (TSPO) lack specificity. In an aspect, the disclosed radiotracers allow accurate detection of maladaptive immune cells and neuroinflammation in MS using a non-invasive method.

Also disclosed herein is a method of monitoring immune response in a subject in response to a treatment for a disease, the method including at least the steps of:
(a) administering a disclosed radiolabeled compound or pharmaceutical composition to the subject;
(b) performing a positron emission tomography (PET) scan on the subject; and
(c) comparing results of the PET scan to results of an initial PET scan of the subject, wherein the initial PET scan was conducted prior to disease treatment or at an earlier stage of disease treatment.

In a further aspect, the disease is associated with increased GPR84 expression. In yet another aspect, the disease can be selected from Alzheimer's disease, multiple sclerosis, glioblastoma, brain metastases, stroke, central nervous system (CNS) injury, a psychiatric disorder, multiple sclerosis, ulcerative colitis, another inflammatory bowel disease, atherosclerosis, pulmonary fibrosis, arthritis, neuropathic pain or other types of pain, chronic fatigue syndrome, fibromyalgia, a bacterial or viral infection, cancer, or any combination thereof.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound,” “a subject,” or “a radioisotope,” includes, but is not limited to, mixtures, combinations, or groups of two or more such compounds, subjects, or radioisotopes, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a tracer refers to an amount that is sufficient to achieve the desired result. The specific level in terms of wt % in a composition required as an effective amount will depend upon a variety of factors including the weight and species of the subject to be imaged, method of administration, presence of excipients or carriers in a composition containing the tracer, and particular condition and/or organ being imaged.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

### Pharmaceutical Compositions

As used herein, “administering” can refer to an administration that is a form of injection such as, for example, intramuscular or parenteral. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for intravenous injection or parenteral administration. Carriers include solids or liquids, and the type of carrier is chosen based on the type of administration being used. The compounds may be administered as a dosage that has a known quantity of the compound.

In order to enhance the solubility and/or the stability of a disclosed compound in a disclosed parenteral injection form or intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions, or suspensions, including saline and buffered media. Parenteral vehicles can include mannitol, normal serum albumin, sodium chloride solution, Ringer's dextrose, dextrose and sodium chloride, lactated Ringer's, and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

### Pharmaceutical Compositions

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof. The effect can be therapeutic in terms of a partial or complete cure of a disease, condition, symptom or adverse effect attributed to the disease, disorder, or condition. The term “treatment” as used herein can include any treatment of a disease causing inflammation associated with increased GPR84 expression in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. In an aspect, administering a disclosed compound and following with PET scan can monitor the success of treatment. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration intravenously or by injection.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In various aspects, a disclosed compound comprising an acidic group or moiety, e.g., a carboxylic acid group, can be used to prepare a pharmaceutically acceptable salt. For example, such a disclosed compound may comprise an isolation step comprising treatment with a suitable inorganic or organic base. In some cases, it may be desirable in practice to initially isolate a compound from the reaction mixture as a pharmaceutically unacceptable salt and then simply convert the latter back to the free acid compound by treatment with an acidic reagent, and subsequently convert the free acid to a pharmaceutically acceptable base addition salt. These base addition salts can be readily prepared using conventional techniques, e.g., by treating the corresponding acidic compounds with an aqueous solution containing the desired pharmacologically acceptable cations and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before.

Bases which can be used to prepare the pharmaceutically acceptable base-addition salts of the base compounds are those which can form non-toxic base-addition salts, i.e., salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucamine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylamine, ethylamine, propylamine, isopropylamine, any of the four butylamine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, triethylamine, tripropylamine, tromethamine, 2-diethylaminoethanol, 2-dimethylaminoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

In various aspects, a disclosed compound comprising a protonatable group or moiety, e.g., an amino group, can be used to prepare a pharmaceutically acceptable salt. For example, such a disclosed compound may comprise an isolation step comprising treatment with a suitable inorganic or organic acid. In some cases, it may be desirable in practice to initially isolate a compound from the reaction mixture as a pharmaceutically unacceptable salt and then simply convert the latter back to the free base compound by treatment with an basoc reagent, and subsequently convert the free base to a pharmaceutically acceptable acid addition salt. These acid addition salts can be readily prepared using conventional techniques, e.g., by treating the corresponding basic compounds with an aqueous solution containing the desired pharmacologically acceptable anions and then evaporating the resulting solution to dryness, preferably under reduced pressure. Alternatively, they also can be prepared by treating the free base form of the disclosed compound with a suitable pharmaceutically acceptable non-toxic inorganic or organic acid.

Acids that can be used to prepare the pharmaceutically acceptable acid-addition salts of the base compounds are those which can form non-toxic acid-addition salts, i.e., salts containing pharmacologically acceptable anions formed from their corresponding inorganic and organic acids. Exemplary, but non-limiting, inorganic acids include hydrochloric hydrobromic, sulfuric, nitric, phosphoric and the like. Exemplary, but non-limiting, organic acids include acetic, benzenesulfonic, benzoic, camphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt comprises an anion formed from hydrobromic, hydrochloric, maleic, phosphoric, sulfuric, and tartaric acids.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

In various aspects, a disclosed parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure are suitable for injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions, or suspensions, including saline and buffered media. Parenteral vehicles can include mannitol, normal serum albumin, sodium chloride solution, Ringer's dextrose, dextrose and sodium chloride, lactated Ringer's, and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In an aspect, the disclosed pharmaceutical compositions require minimal processing prior to administration due to the relatively short half lives of the included radioisotopes.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

The present disclosure can be described in accordance with the following numbered aspects, which should not be confused with the claims.

Aspect 1. A radiolabeled compound having a structure of Formula I:

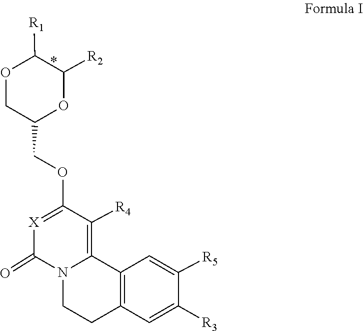

wherein X is N or CH;
wherein R₁ and R₂ are both hydrogen and the bond marked by * is a single bond, or wherein R₁ and R₂ are bridged to form a heteroaromatic ring and the bond marked by * is a component of the heteroaromatic ring;
wherein R₃ comprises a halogen, C1-C4 alkyl ether, or fluorosulfonate group;
wherein R₃ comprises at least one radioisotope; and
wherein R₄ and R₅ are independently hydrogen or methyl.

Aspect 2. The radiolabeled compound of aspect 1, wherein the at least one radioisotope is selected from ¹¹C and ¹³F.

Aspect 3. The radiolabeled compound of aspect 1 or 2, wherein X is N.

Aspect 4. The radiolabeled compound of aspect 1 or 2, wherein X is CH.

Aspect 5. The radiolabeled compound of any one of aspects 1-4, wherein R₁ and R₂ are both hydrogen and the bond marked by * is a single bond.

Aspect 6. The radiolabeled compound of any one of aspects 1-4, wherein the radiolabeled compound has the structure

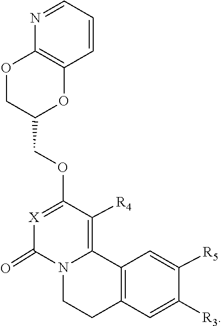

Aspect 7. The radiolabeled compound of any one of aspects 1-6, wherein R₄ and R₅ are methyl.

Aspect 8. The radiolabeled compound of any one of aspects 1-6, wherein R₄ and R₅ are hydrogen.

Aspect 9. The radiolabeled compound of any one of aspects 1-8, wherein R₃ comprises at least one ¹¹C atom.

Aspect 10. The radiolabeled compound of any one of aspects 1-8, wherein R₃ comprises at least one ¹⁸F atom.

Aspect 11. The radiolabeled compound of any one of aspects 1-10, wherein R₃ is selected from

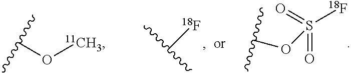

Aspect 12. The radiolabeled compound of any one of aspects 1-11, wherein the radiolabeled compound is selected from

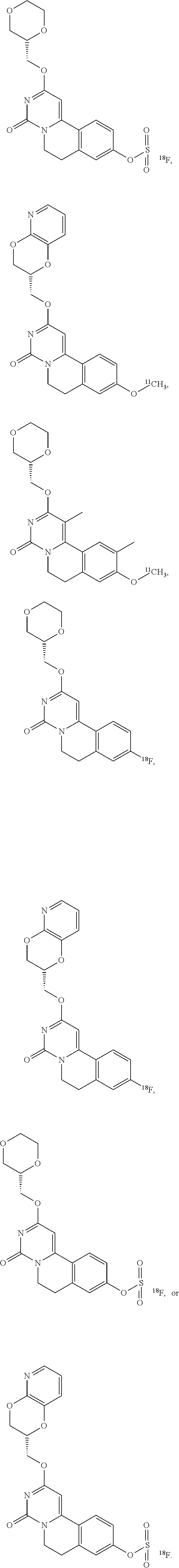

Aspect 13. The radiolabeled compound of any one of aspects 1-12, wherein the radiolabeled compound undergoes beta plus decay and emits a positron.

Aspect 14. The radiolabeled compound of any one of aspects 1-13, wherein the at least one radioisotope has a half life of at least about 20 minutes.

Aspect 15. The radiolabeled compound of any one of aspects 1-13, wherein the at least one radioisotope has a half life of at least about 100 minutes.

Aspect 16. The radiolabeled compound of any one of aspects 1-15, wherein the radiolabeled compound is capable of penetrating an intact blood-brain barrier (BBB).

Aspect 17. A pharmaceutical composition comprising the radiolabeled compound of any one of aspects 1-16.

Aspect 18. The pharmaceutical composition of aspect 17, further comprising at least one excipient or carrier.

Aspect 19. A method for imaging inflammation in a subject, the method comprising:
(a) administering the radiolabeled compound of any one of aspects 1-16 or the pharmaceutical composition of aspect 17 or 18 to the subject; and
(b) performing positron emission tomography (PET) on the subject.

Aspect 20. The method of aspect 19, wherein the inflammation is associated with increased GPR84 expression relative to a level of GPR84 expression in a subject not experiencing inflammation.

Aspect 21. The method of aspect 19 or 20, wherein the inflammation comprises neuroinflammation, gut inflammation, or any combination thereof.

Aspect 22. The method of any one of aspects 19-21, wherein the radiolabeled compound binds to GPR84.

Aspect 23. The method of any one of aspects 19-22, wherein the subject is a mammal.

Aspect 24. The method of aspect 23, wherein the mammal is a human, mouse, rat, hamster, rabbit, guinea pig, dog, cat, horse, cattle, sheep, swine, or non-human primate.

Aspect 25. A method for determining a stage of a disease in a subject, the method comprising:
(a) administering the radiolabeled compound of any one of aspects 1-16 or the pharmaceutical composition of aspect 17 or 18 to the subject;
(b) performing a positron emission tomography (PET) scan on the subject; and
(c) comparing results of the PET scan to one or more standard PET scans.

Aspect 26. The method of aspect 25, wherein the disease is associated with increased GPR84 expression.

Aspect 27. The method of aspect 26, wherein the disease comprises Alzheimer's disease, multiple sclerosis, glioblastoma, brain metastases, stroke, central nervous system (CNS) injury, a psychiatric disorder, multiple sclerosis, ulcerative colitis, another inflammatory bowel disease, atherosclerosis, pulmonary fibrosis, arthritis, neuropathic pain or other types of pain, chronic fatigue syndrome, fibromyalgia, a bacterial or viral infection, cancer, or any combination thereof.

Aspect 28. A method of monitoring immune response in a subject in response to a treatment for a disease, the method comprising:
(a) administering the radiolabeled compound of any one of aspects 1-16 or the pharmaceutical composition of aspect 17 or 18 to the subject;
(b) performing a positron emission tomography (PET) scan on the subject; and
(c) comparing results of the PET scan to results of an initial PET scan of the subject, wherein the initial PET scan was conducted prior to disease treatment or at an earlier stage of disease treatment.

Aspect 29. The method of aspect 28, wherein the disease is associated with increased GPR84 expression.

Aspect 30. The method of aspect 29, wherein the disease comprises Alzheimer's disease, multiple sclerosis, glioblastoma, brain metastases, stroke, central nervous system (CNS) injury, a psychiatric disorder, multiple sclerosis, ulcerative colitis, another inflammatory bowel disease, atherosclerosis, pulmonary fibrosis, arthritis, neuropathic pain or other types of pain, chronic fatigue syndrome, fibromyalgia, a bacterial or viral infection, cancer, or any combination thereof.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Development and Testing of ¹¹C-GLPG38

### Machine Learning Methods

A data set of 140 successful and unsuccessful CNS tracers was compiled in order to develop and evaluate a novel scoring algorithm (CNS penetrance optimization program [CNSPOP]) and 15 ML approaches for predicting CNS uptake. CNSPOP was applied to all 140 tracers and ML approaches were assessed using a 60:40 training:test split to determine sensitivity, specificity, and positive/negative predictive values (PPV/NPV, respectively). This computational approach was subsequently employed to identify GPR84 antagonist GLPG38 as a promising candidate for radiolabeling and use as a CNS PET tracer.

### Identification of GLPG38 as a Candidate PET Radiotracer

Based on encouraging initial biomarker validation, 683 known allosteric GPR84 antagonists199,207-211,213 were reviewed in order to identify promising CNS PET radiotracer candidates using an approach based on published PET MPO methods, and an early precursor to CNSPOP methods described in Chapter 3. Briefly, 683 small molecule antagonists were first sorted by affinity, excluding molecules with an IC50>100 nm. The remaining 409 molecules were then scored using the CNS MPO computational tool137 and reviewed individually to exclude any molecules with a C log P or C log D<1.5. Of the remaining 110 molecules deemed to have sufficient affinity and CNS MPO score, 66 were determined to be amenable to radiolabeling with either C-11 or F-18. Finally, I selected the molecule with the highest CNS MPO score (CNS MPO score 5.9/6, CNS PET MPO score 5/6), in addition to having the best combination of physicochemical parameters (i.e., C log P 2.40, tPSA 57.23, MW 371.43, 0 HBD, no ionizable sites, charge of 0), based on methods I developed and described in Chapter 3. The most promising molecule, GLPG38, was then radiolabeled with C-11 (detailed below) for assessment as a GPR84 PET radiotracer candidate. Notably, radiolabeling based on this initial assessment took place in parallel to development of the final CNSPOP algorithm (see Chapter 3), precluding screening with the final validated algorithm prior to radiosynthesis. However, the CNSPOP score for GLPG38 was retrospectively calculated; the resulting value of 3.125/5 was consistent with a PPV>90% and extremely encouraging for in vivo imaging and CNS uptake.

A synthetic approach for synthesis of a GLPG38 reference standard and synthetic precursor for radiolabeling is presented in Scheme 1:

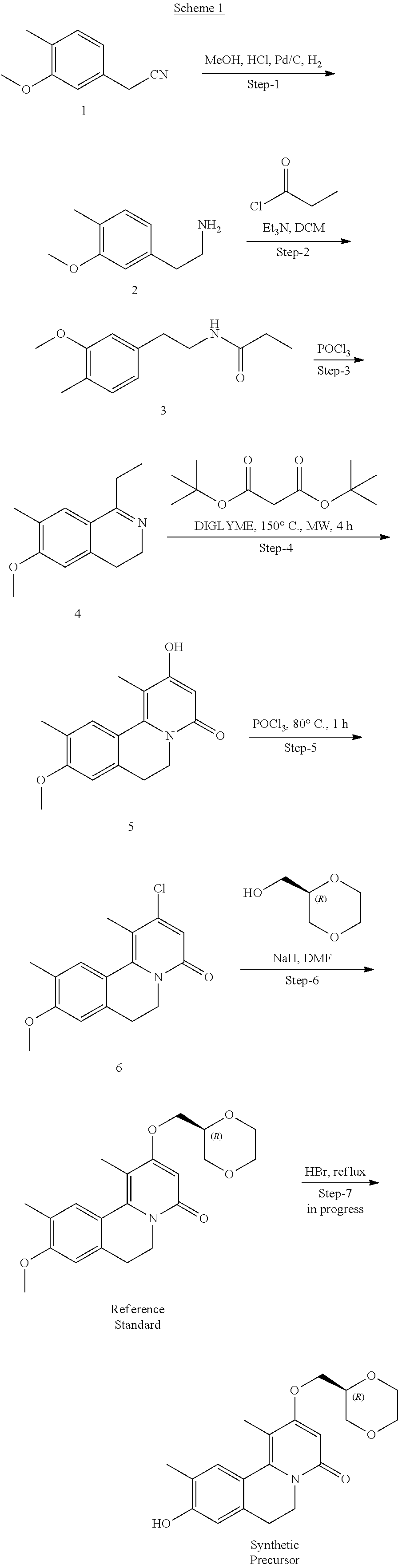

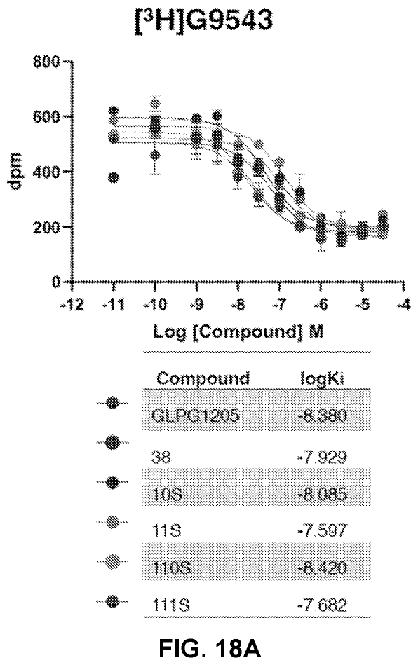

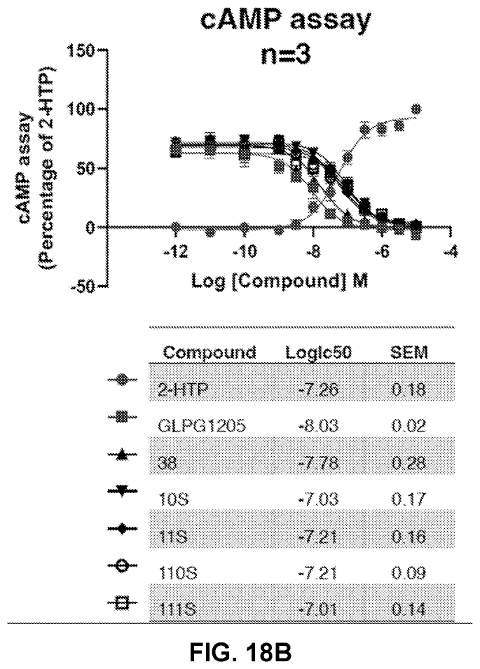

The precursor and reference standard for GLPG38 were synthesized by Jubilant Biosys in sufficient quantity for characterization, radiolabeling, and imaging studies (Scheme 1). To determine affinity of GLPG38 for human GPR84, GLPG38 was assessed alongside other small molecule antagonists, and clinical GPR84 therapeutic GLPG1205, in a [³H]G9543 competitive binding assay and cAMP inhibition assay (FIGS. 18A-18B). Based on the [³H]G9543 assay, the K_{d} of GLPG38 was determined to be 11.77 nM, which falls within the low nanomolar range that is required for in vivo PET imaging studies.

### Radiosynthesis of [¹¹C]GLPG38

Precursor (1.0-1.25 mg) was dissolved in DMF (0.5 mL) and loaded into a glass reactor. Care was taken not to introduce base until just prior to delivery of Mel to the reactor, as addition of base too early leads to drastic decrease of yield and drives formation of a radioactive side product over the desired methylated product (Chapter 4 supplemental information). 5.0 M KOH in water (1.2 μL) was added to the reactor just prior to the delivery of [¹¹C]Mel and the reactor was cooled to −10° C.; the cell was then sealed and the reaction heated to 70° C. for 5 minutes. The reaction mixture was cooled to 40° C. and diluted with 1 mL of 40:60 H₂O MeCN. The crude reaction mixture was purified via semipreparative HPLC (column: Phenomenex Gemini 5 mm C18 110 Å, 250×10 mm; mobile phase A: H₂O with 0.1% trifluoroacetic acid (TFA) by volume; mobile phase B: MeCN with 0.1% TFA by volume; program: 40-60% B in 15 min at 5 mL/min; standard retention time: 8.7 min) to furnish radiochemically pure [¹¹C]GLPG38. Pure product was collected in a round-bottom tube containing 25 mL H2O. The solution was passed through a Sep-pak plus lite C18 (conditioned with 5 mL EtOH and 10 mL H₂O) to trap product, and rinsed with H₂O (10 mL). Pure [¹¹C]GLPG38 product was eluted with EtOH (0.5 mL) formulated in saline (4.5 mL). Purity and identity of final product were confirmed via analytical HPLC (column: Phenomenex Gemini 5 mm C18 110 Å, 250×4.6 mm; mobile phase A: H₂O with 0.1% TFA by volume; mobile phase B: MeCN with 0.1% TFA by volume; program: 50-95% B in 15 minutes at 1 mL/min; standard retention time: 6.78 minutes).

Synthesis of [¹¹C]GLPG38 is shown in Scheme 2:

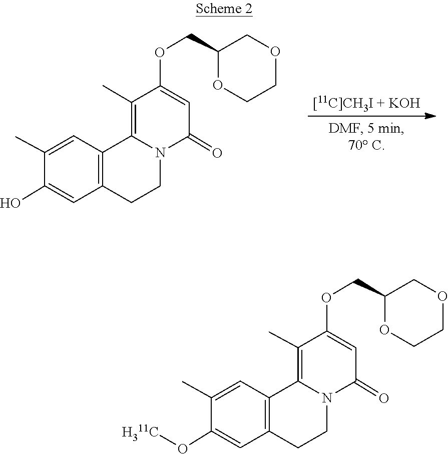 [SMILES: Cc1cc2c(cc1O)CCn1c-2c(C)c(OC[C@H]2COCCO2)cc1=O ; Cc1cc2c(cc1O[11CH3])CCn1c-2c(C)c(OC[C@H]2COCCO2)cc1=O]

### In Vitro Radioactive Cell Binding Studies

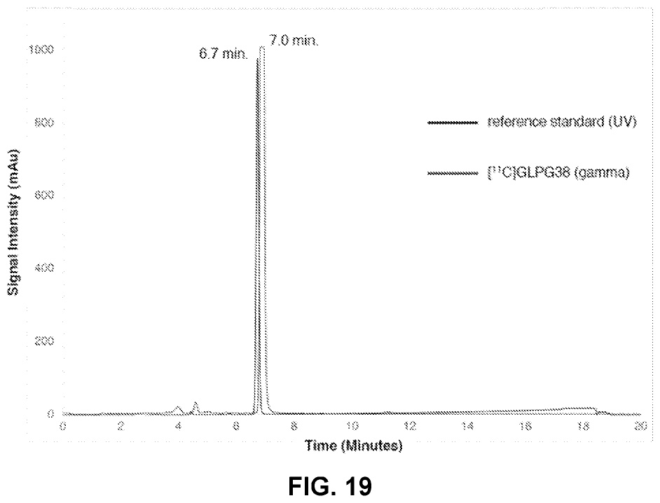

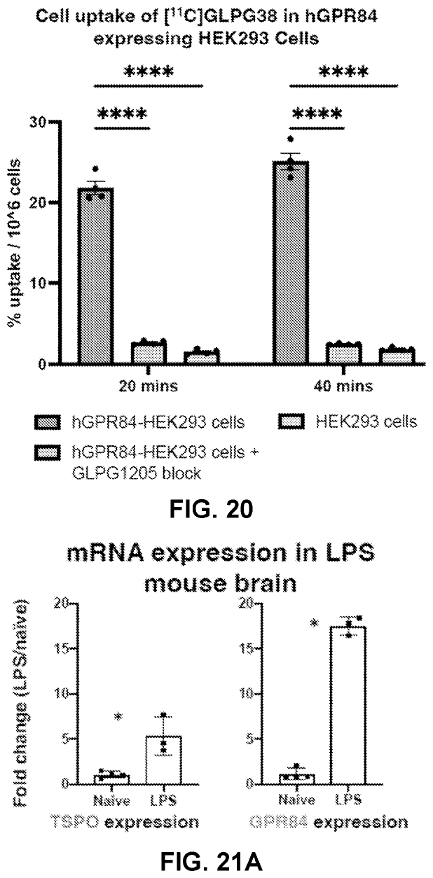

Based on this encouraging preliminary data, [¹¹C]GLPG38 was synthesized via C-11 methylation using [¹¹C]Mel in suitable RCY (8.13%±3.30%), purity, and molar activity (395 mCi/μmol±362 mCi/μmol) for preliminary cell uptake and imaging studies (n=3) (FIGS. 18A-18B, 19). To assess binding specificity, [¹¹C]GLPG38 was incubated for 40 minutes with hGPR84 expressing HEK293 cells, parental control HEK293 cells, or hGPR84 expressing HEK293 cells in the presence of GPR84 inhibitor GLPG1205 as a competitive blocking agent. Results from these studies demonstrated significantly increased (p<0.0001) [¹¹C]GLPG38 binding to hGPR84 expressing HEK293 cells relative to control cells, reflected by a 13.5-fold higher binding in hGPR84⁺HEK293 cells compared to parental control HEK293 cells. Similarly, incubation with hGPR84⁺HEK293 cells in the presence of blocking with GLPG1205 resulted in a 90% reduction (p<0.001) in signal at 40 minutes (FIG. 20).

GPR84-transduced HEK-293 cells and control HEK-293 cells were plated at a concentration of 3×10⁵ cells per well in media (DMEM with 10% FBS and 1% anti-anti) into three 12-well plates 16-24 hr prior to the cell binding assay. Once cells reached ˜90% confluency, the radiotracer was prepared. Additionally, a blocking solution was prepared of 35 μM GLPG1205 in DMSO, and DMEM media warmed. The radiotracer was diluted in DMEM without FBS or antibiotic to yield a 30 mL solution where the amount of activity was 25 μCi in 500 μL. The first 12-well plate was incubated for 20 minutes at 37° C. and 5% CO₂ in air while the next was incubated for 40 minutes at the same conditions. Cells were subsequently washed with PBS, trypsinized, resuspended in DMEM+10% FBS, and pipetted into gamma counting tubes at 500 μL volume. Radioactivity levels bound to cells were measured in a gamma counter (Hidex Oy, Helsinki, Finland), and cell counts were recorded using Trypan blue and an automatic cell counter to normalize radioactivity levels to cell count.

### In Vivo Mouse Imaging

Animals: All experiments involving animals were in accordance with the Stanford Administrative Panel on Laboratory Animal Care (APLAC), accredited by the Association for the Assessment and Accreditation of Laboratory Animal Care (AAALAC). 3-4 mo female C57BL/6J mice were purchased from Jackson Laboratories (Jax) and were acclimated to the facility for at least 2 weeks prior to experimentation. The mice were in a temperature-controlled environment with a 12 hr light/dark schedule, and provided unrestricted access to food and water.

Dynamic PET/CT Imaging and Analysis of [¹¹C]GLPG38 in Mice: Healthy female mice (n=4) were placed in a 2×2 mouse holder and loaded into an Inveon dPET (Siemens) for 60 minute dynamic PET imaging. Data were acquired in list mode format over 60 minutes (4×15 second frames, 4×1 minute frames, 11×5 minute frames) commencing just prior to tail-vein injection of radiotracer (average 346 μCi) and reconstructed via OSEM-3D with scatter correction. A transmission scan was acquired to correct for attenuation during image reconstruction. Following PET imaging, mice were transferred to a SOFIE GNEXT PET/CT for CT acquisition. PET/CT data were co-registered and analyzed using Inveon Research Workplace software to generate images and quantify radiotracer uptake in regions of interest (ROIs). A 3D ROI was manually defined for the whole brain using CT data as anatomical reference, then applied to the full dynamic data set to generate a time activity curve (TAC).

Imaging studies were performed on a Concorde P4 microPET (Knoxville, TN) in an intact, mature female rhesus monkey (n=1, animal weight 9.36 kg). The animal was anesthetized in the home cage with Telazol and transported to the PET facility, where the subject was intubated for mechanical ventilation; anesthesia was continued with isoflurane and maintained throughout the duration of the PET scan. A venous catheter was inserted into one hind limb and the monkey was placed on the PET gantry with its head secured to prevent motion artifacts. Ten minutes later, 5.19 mCi of the radiotracer of interest was injected intravenously and respiratory rate was collected for the entire duration of the scan. Data were corrected for attenuation and scatter and reconstructed using the three-dimensional-maximum a priori method (3D MAP algorithm). A ROI was defined for the whole brain on multiple planes by using a summed image. The volumetric ROI was then applied to a full dynamic data set to generate a TAC.

### PET Imaging of Rhesus Macaque: General Considerations

Primate studies were conducted in accordance with the standards set by the University of Michigan Institutional Animal Care & Use Committee (IACUC), which is accredited by the Association for the Assessment and Accreditation of Laboratory Animal Care (AAALAC International). The University of Michigan PET Center has maintained 2 rhesus macaques for-19 years and the monkeys are individually housed in adjacent steel cages (83.3 cm high×152.4 cm wide×78.8 cm deep) equipped with foraging boxes. They are currently housed in adjacent cages as repeated attempts to socially house them in the same cage have been unsuccessful due to aggressive incompatibility. Cages are metal and contain gridded floors for radiation safety reasons (radioactive waste is contained to the gridded floor and is easier to clean). Temperature and humidity are carefully controlled, and the monkeys are kept on a 12 hr light/12 hr dark schedule. Monkeys are fed Lab Fiber Plus Monkey Diet (PMI Nutrition Intl. LLC, Shoreview MN, USA) that is supplemented with fresh fruit and vegetables daily. Water and enrichment toys (manipulanda and food-based treats) are available continuously in the home cage.

### PET Imaging and Analysis of [¹¹C]GLPG38 in Rhesus Macaque: Imaging Studies

Imaging studies were performed on a Concorde P4 microPET (Knoxville, TN) in an intact, mature female rhesus monkey (n=1, animal weight 9.36 kg). The animal was anesthetized in the home cage with Telazol and transported to the PET facility, where the subject was intubated for mechanical ventilation; anesthesia was continued with isoflurane and maintained throughout the duration of the PET scan. A venous catheter was inserted into one hind limb and the monkey was placed on the PET gantry with its head secured to prevent motion artifacts. Ten minutes later, 5.19 mCi of [¹¹C]GLPG38 was administered in a bolus dose over 1 minute, and the brain imaged for 60 minutes (5×1 min frames, 2×2.5 min frames, 2×5 min frames, 4×10 min frames). Emission data were collected beginning with the injection and continued for 60 minutes. Vitals (HR, SPO2, EtCO2 and respiratory rate) were collected for the entire duration of the scan. Data were corrected for attenuation and scatter and reconstructed using the three-dimensional-maximum a priori method (3D MAP algorithm). A ROI was defined for the whole brain on multiple planes by using a summed image. The volumetric ROI was then applied to a full dynamic data set to generate a TAC.

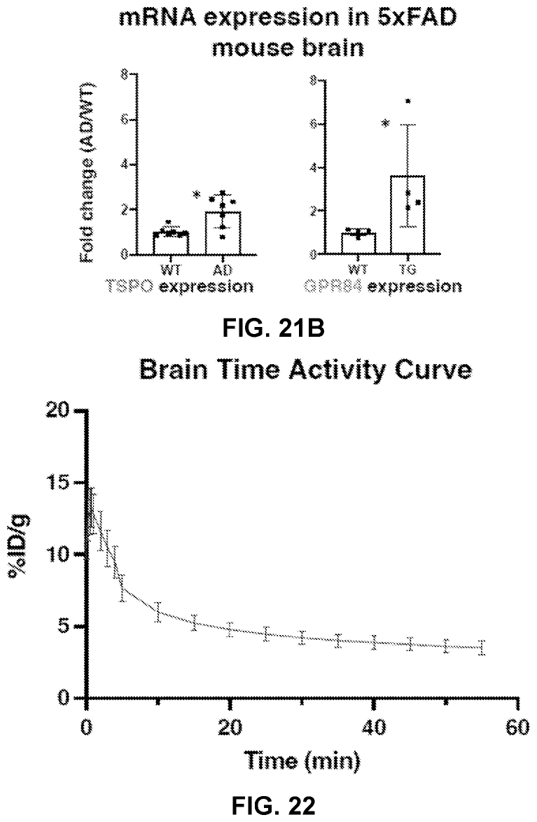

qPCR analysis of GPR84 gene expression in brains from two murine disease models known to have a significant neuroinflammatory component, LPS-induced sepsis and the AD model 5×FAD transgenic mice, demonstrated significant upregulation of GPR84 compared to naïve/wild-type control mice (FIGS. 21A-21B). Importantly, changes in GPR84 expression between background and disease were higher compared to TSPO in both LPS (approx. 15-fold change for GPR84 vs 5-fold for TSPO) and 5×FAD (approx. 4-fold change for GPR84 vs 2-fold for TSPO) mouse brains, encouraging the use of GPR84 as a PET biomarker.

### In Vivo Imaging of [¹¹C]GLPG38 in Mice and Rhesus Macaque

After confirming high affinity and binding specificity, [¹¹C]GLPG38 was administered to naïve wild-type mice to assess in vivo kinetics and uptake. PET images and the corresponding time activity curve (TAC) showed excellent CNS uptake of [¹¹C]GLPG38 (uptake 9.46% ID/g at 4 minutes post injection) and appropriate washout to 3.53% ID/g in healthy mice, consistent with what is known about low basal GPR84 receptor expression (FIG. 22).

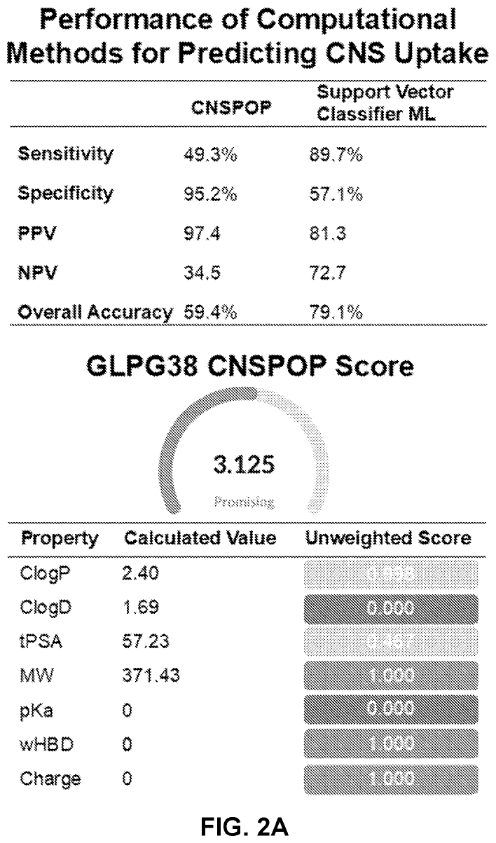

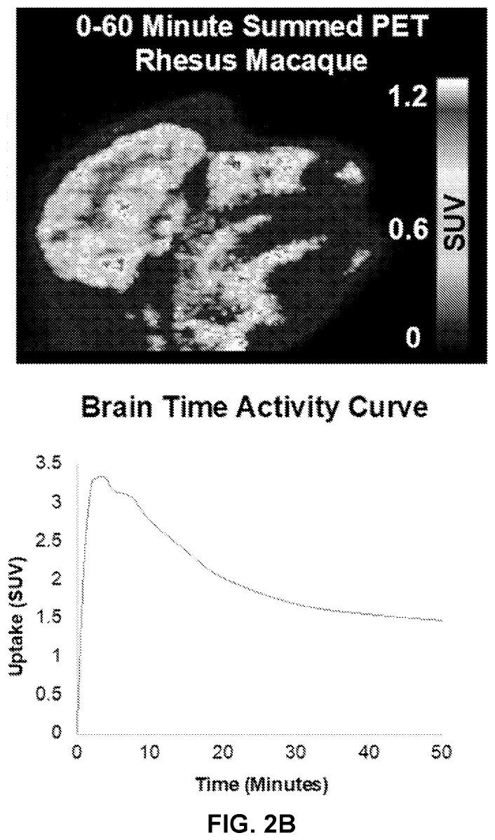

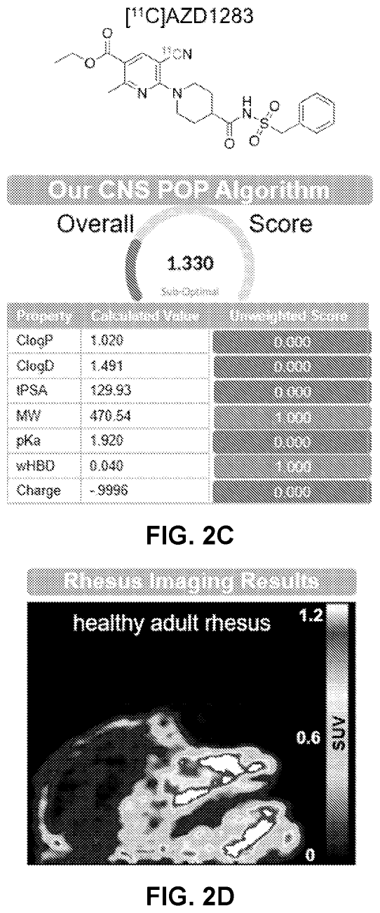

Mouse imaging studies and murine models of disease are an essential aspect of evaluating and assessing the translational potential of novel PET biomarkers and radiotracers; however, significant inter-species differences in overall physiology (e.g. metabolism, efflux pumps at the BBB) mean that imaging in higher-order species such as non-human primates is a more accurate predictor of radiotracer uptake in humans. To more accurately predict uptake and kinetics of [¹¹C]GLPG38 in humans and overall translational potential, a single healthy adult Rhesus macaque was administered [¹¹C]GLPG38 intravenously and underwent a 60 minute dynamic PET scan (FIG. 2B). Imaging and a TAC demonstrated high initial uptake of [¹¹C]GLPG38 into the healthy brain with a peak uptake of 3.3 SUV at 4 minutes post injection and appropriate washout over the course of 60 min to a level of 1.47 SUV. Collectively, preliminary data show that [¹¹C]GLPG38 is a highly CNS penetrant PET radiotracer with high affinity for GPR84.

### Dissociation Constant Determination and Imaging of an LPS Model

To determine K_{D} of GLPG38, a competitive binding assay was performed using a membrane preparation from cells expressing GPR84-Gi in addition to [³H]G9543, a known GPR84 antagonist. [¹¹C]GLPG38 was then radiosynthesized via [¹¹C]methylation of desmethyl precursor using [¹¹C]methyl iodide. Binding specificity of [¹¹C]GLPG38 was assessed using human GPR84-stably-expressing human embryonic kidney cells (HEK-293) versus control HEK-293 cells. For animal studies, sepsis was induced in female C57BL/6J mice via intraperitoneal injection of lipopolysaccharide (LPS, 10 mg/kg); vehicle-treated mice were injected with saline alone. Dynamic 60 min PET/CT imaging was performed 24 hr following LPS-administration using [¹¹C]GLPG38. Subsequently, mice were perfused with PBS to remove blood, and radioactive signal was assessed in CNS and peripheral tissues via gamma counting. Ex vivo autoradiography of sagittal mouse brain sections was conducted to obtain high resolution images of [¹¹C]GLPG38 binding. To investigate alterations in GPR84 mRNA expression, quantitative polymerase-chain reaction was performed using mouse brain tissue and human monocyte-derived macrophage cells stimulated with LPS 24 hr prior to RNA extraction.

The affinity of GLPG38 for GPR84 was found to be low nanomolar (11.77 nM), indicating its potential as a PET tracer. [¹¹C]GLPG38 was synthesized in sufficient radiochemical yield (8.13%±3.30%, n=3) and >95% radiochemical purity. Cell binding studies demonstrated 13.5-fold higher binding of [¹¹C]GLPG38 to hGPR84-HEK-293 cells compared to control cells (p<0.0001, n=4). Co-incubation with GPR84 antagonist (GLPG1205, 35 μM) reduced tracer binding in hGPR84-HEK293 cells by >90% (p<0.0001, n=4) demonstrating high specificity of [¹¹C]GLPG38. Dynamic mouse PET imaging revealed significantly elevated [¹¹C]GLPG38 signal in the brain of LPS-treated mice compared to those treated with saline at 40-60 min post-tracer injection (p<0.05). Autoradiography of brain sections from LPS-treated mice demonstrated a marked increase in [¹¹C]GLPG38 binding compared to vehicles, with substantial reduction following pre-blocking with GLPG1205 (5 mg/kg). Gamma counting confirmed imaging findings, with increased signal in the brain (p=0.0023) of LPS-injected mice, indicating the utility of [¹¹C]GLPG38 for detecting neuroinflammation known to occur in this model. A significant increase in tracer binding to adipose tissue from LPS mice was also observed (p=0.007), suggesting [¹¹C]GLPG38 could be used to quantify known myeloid-driven inflammation in this tissue in LPS models. Importantly, GPR84 mRNA expression was found to be more robustly upregulated in both LPS mouse brain tissue and human cells treated with LPS compared to translocator protein (18 kDa) (TSPO), a widely-evaluated PET biomarker of inflammation.

[¹¹C]GLPG38 is a promising and specific PET tracer for detecting innate immune activation. Since GPR84 is more specific than TSPO as a biomarker, GPR84-PET has the potential to enable more accurate characterization of myeloid cell-specific inflammation in the CNS and beyond in numerous diseases.

### Ulcerative Colitis Model

To investigate alterations in GPR84 mRNA expression under pro-inflammatory conditions, quantitative polymerase-chain reaction was performed using mouse brain and human monocyte-derived macrophage cells (hMDMs) treated with lipopolysaccharides (LPS) 24 hr prior to RNA extraction. [¹¹C]GLPG38 and [¹⁸F]MGX-110S were made via [¹¹C]methylation of the desmethyl precursor and by copper mediated [¹⁸F]fluorination of Bpin precursor respectively. Binding specificities of both tracers were assessed using human GPR84-stably-expressing human embryonic kidney cells (HEK-293) versus control HEK-293 cells. [¹¹C]GLPG38 binding was also assessed in hMDMs+/−treatment with LPS. Dynamic PET/CT imaging of healthy mice was commenced just prior to injection of [¹¹C]GLPG38 (100-150 μCi) or [¹⁸F]MGX-110S (60-120 μCi) to determine biodistribution and kinetics. In vitro autoradiography (ARG) of human colon sections (5 μm) was conducted to test the ability of each tracer to detect innate immune activation in active UC.

GPR84 mRNA expression was more robustly upregulated in both LPS mouse brain tissue and human cells treated with LPS compared to translocator protein (18 kDa) (TSPO), a widely-evaluated PET biomarker of inflammation. [¹¹C]GLPG38 was synthesized in 8.13%±3.30% radiochemical yield (n=3) and >95% radiochemical purity (RCP). [¹⁸F]MGX-110S was generated in 22.21±11.99% yield (n=3) and >99% RCP. Cell binding studies showed 13.5-fold higher binding of [¹¹C]GLPG38 and 33.4-fold higher [¹⁸F]MGX-110S binding to hGPR84-HEK-293 cells vs control cells (p<0.0001, n=4). Co-incubation with GPR84 antagonist (GLPG1205, 1000× by mass) reduced tracer binding in hGPR84-HEK293 cells by >90% (p<0.0001, n=4) proving high specificity of these tracers in cells. Notably, hMDMs challenged with LPS (100 ng/mL) stimulus were found to display significantly higher binding of [¹¹C]GPLG38 compared to unstimulated cells, and this signal was reduced to baseline following co-incubation with GLPG1205. Whole-body PET images of healthy mice depict relatively low background signal in most peripheral tissues, highlighting their potential for imaging inflammation throughout the body. The highest signal (though lower for [¹⁸F]MGX-110S) was observed in liver and gallbladder indicating biliary excretion. In the context of human inflammatory disease, in vitro ARG of [¹¹C]GLPG38 and [¹⁸F]MGX-110S in active UC colon tissue samples demonstrated marked increase of binding for both tracers compared to age-, sex-, and ethnicity-matched healthy samples, and this signal was markedly attenuated by pre-blocking with 1000×GLPG1205 by mass. Regions of high tracer binding corresponded with immune infiltrates shown by H&E staining.

Both PET tracers enable specific detection of GPR84 and innate immune activation in cells and UC tissue. [¹⁸F]MGX-110S shows high potential for translation given its favorable half-life, in vivo distribution, and specificity for GPR84.

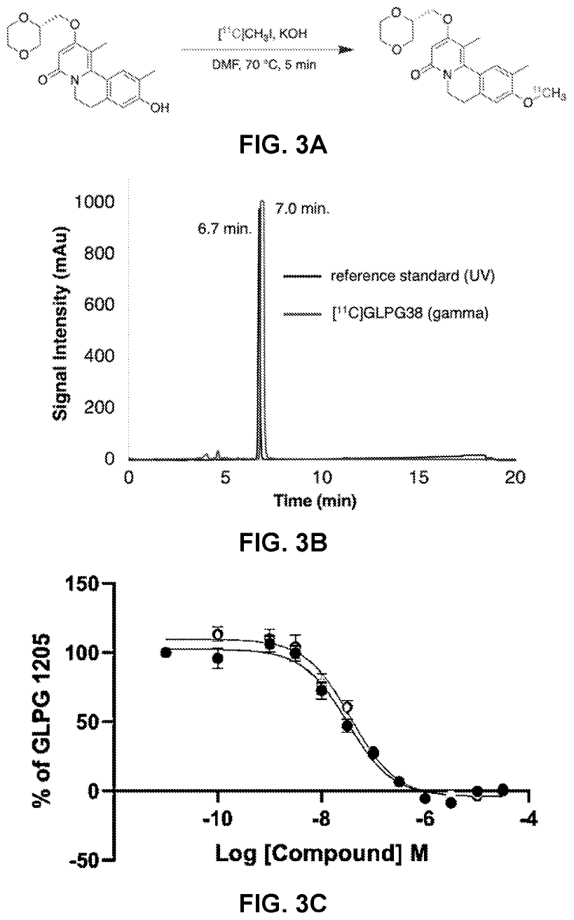

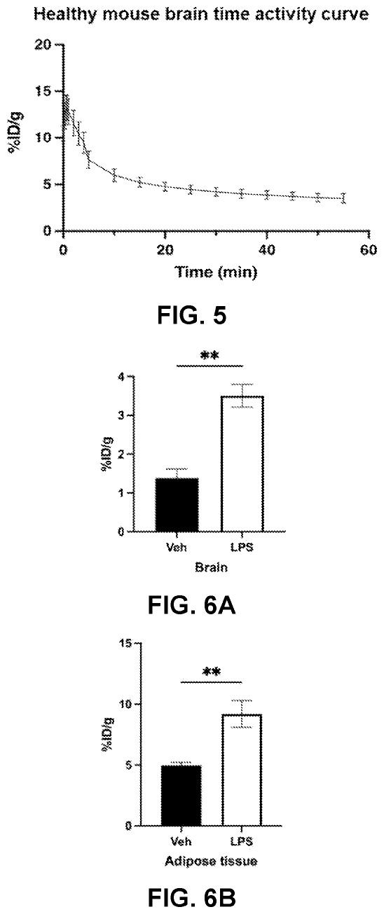

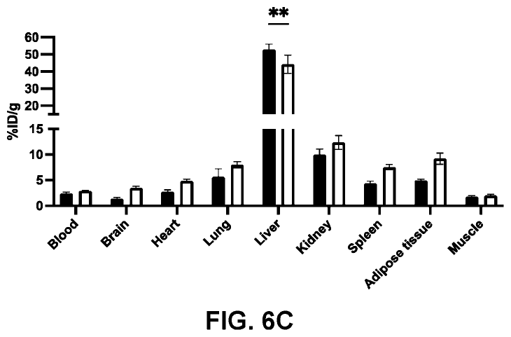

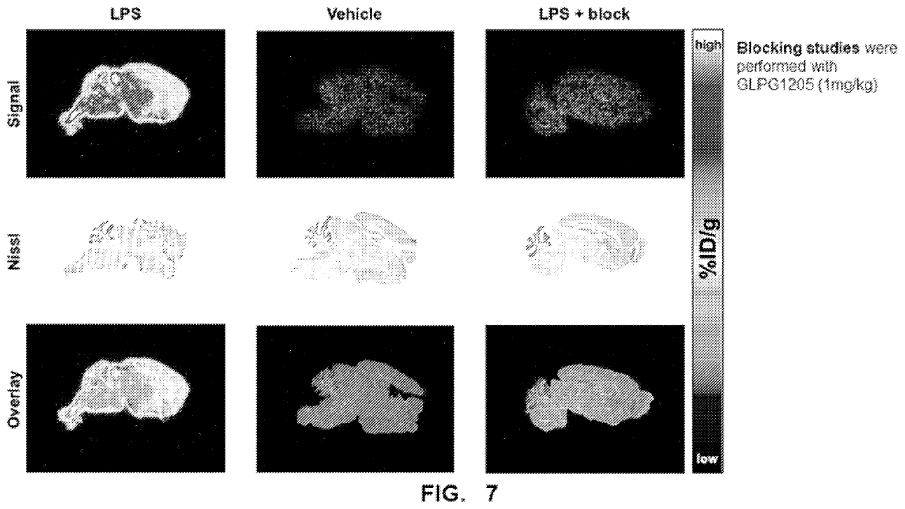

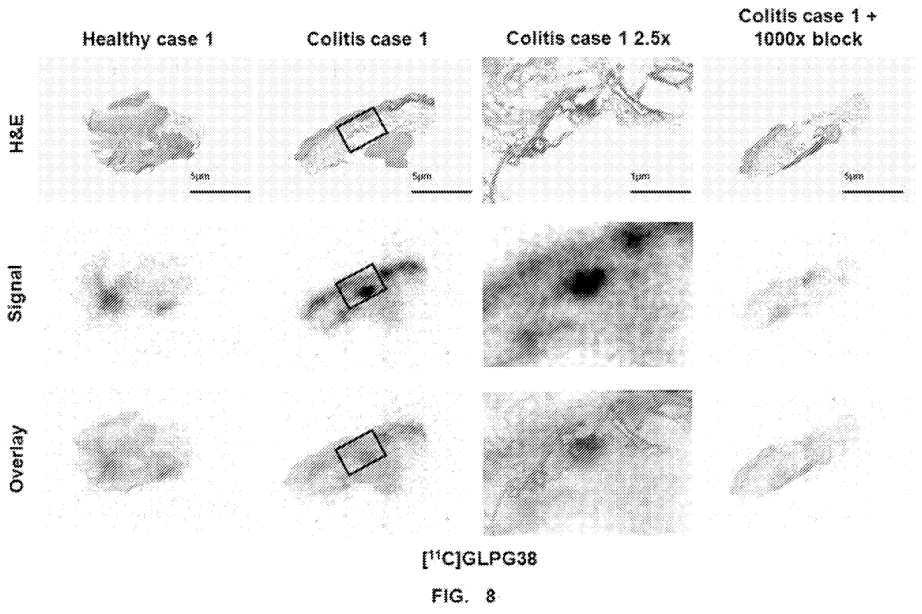

Additional data for [¹¹C]GLPG38 is presented in FIGS. 3A-7.

### Results and Conclusion

Here, a rational, systematic approach was applied to identify GLPG38 as a promising first-in-class PET radiotracer suitable for in vivo imaging of GPR84. GLPG38 demonstrated high affinity and suitable specificity for human GPR84 based on in vitro studies, motivating radiolabeling and in vivo imaging. [¹¹C]GLPG38 was radiolabeled in suitable RCY and molar activity conducive to animal imaging experiments, and demonstrated excellent CNS uptake and appropriate washout in both healthy naïve wildtype mice and rhesus macaque. Cumulatively, these preliminary studies with [¹¹C]GLPG38 are very encouraging and warrant further evaluation and use in vivo. Additionally, these data serve as a promising first application of the described new computation-driven approach to CNS radiotracer design described above. Studies are currently underway to evaluate the ability of [¹¹C]GLPG38 to detect changes in innate immune activation in mice treated with LPS as a murine model of sepsis. Future studies will investigate NSB in mice, as well as binding and NSB in human brain tissue from healthy humans and those with Alzheimer's disease. In parallel, colleagues within the James lab are currently developing a new generation of GPR84 radiotracer candidates amenable to labeling with F-18 as well as C-11.

CNSPOP differentiated permeable from non-permeable CNS PET tracers with excellent PPV (97.4%), and good sensitivity (49.3%) and overall accuracy (59.4%). A support vector ML algorithm displayed improved sensitivity (89.7%) and overall accuracy (79.1%), with good PPV (81.3%), suggesting a combination of these methods for identification of tracer candidates with high likelihood of CNS uptake (FIG. 2A). GPLG38 was identified based on CNSPOP score (3.125/5, corresponding to PPV>90%) and its affinity for GPR84 (11.77 nM), and then radiolabeled in sufficient radiochemical yield (8.13%±3.30%, n=3) with high radiochemical purity (>95%) (FIG. 2A). PET imaging demonstrated robust CNS uptake of [¹¹C]GLPG38 in mice and rhesus macaque (9.46% ID/g and 3.3 SUV respectively at 4 minutes post injection), followed by washout consistent with low basal GPR84 expression in healthy brain (FIG. 2B).

ML and CNSPOP algorithms accurately differentiated known permeable from non-permeable CNS PET tracers with excellent PPV, and enabled identification of a new promising GPR84 tracer candidate. This tracer was successfully synthesized and shown to have robust CNS uptake in mice and rhesus macaque. This work establishes a novel computational approach to predict CNS penetrance of PET tracer candidates and highlights [¹¹C]GLPG38 as a promising new tracer for an emerging neuroinflammation imaging biomarker.

### Example 2: Development and Testing of ¹¹C-MGX-10S and ¹¹C-MGX-11S

### Discussion

An activated immune system amplifies GPR84 production primarily on the myeloid cells. Two ¹¹C labeled PET tracers have been synthesized that are specific for GPR84 in vitro in human-GPR84-expressing HEK293 cells and in vivo in the murine model of MPS induced neuroinflammation. A 2020 publication on negative allosteric modulators of GPR84 guided potential tracer selection. The published literature was modified to fit into the disclosed synthesis of HPLC standards and precursors for ¹¹C labeling. The competitive binding assay with ³H-G9543 (known GPR84 inhibitor) resulted low nanomolar kinetic inhibition constant (K) of MGX-10S (20.41±7.95 nM) and MGX-11S (38.90±6.63 nM). Functional cyclic adenosine 3′,5′-monophosphate (cAMP) assay and the orthogonal (³⁵S-guanosine 5′-O-[γ-thio]triphosphate (GTPγS)) binding assays furnished moderate to high nanomolar half maxima inhibitory concentration (IC₅₀). These properties-strong binding affinity but less potent inhibitory effect-made ¹¹C-MGX-10S and ¹¹C-MGX-11S potential ‘Goldilocks’ PET tracers. In the pursuit of these radiotracers, different bases, base equivalents, and temperatures were screened to determine the best conditions of ¹¹C labeling with high radiochemical purity (>99%) and molar activity (3-6 Ci/μmol). Once optimized, the in vitro specificity of these tracers was tested. The ¹¹C-MGX-10S tracer demonstrated more pronounced specificity than ¹¹C-MGX-11S when compared to the parental HEK293 cells (14.5-fold versus 3.25-fold). When blocked with GLPG1205, both tracers produced a significant reduction in binding (91.7% versus 91.5%). Higher lipophilicity of ¹¹C-MGX-11S likely contribute to the non-specific binding with the parental HEK293 cell membranes. Next, both tracers were compared head-to-head in their blood-brain-barrier permeability. Both tracers were injected into healthy mice (n=4) to determine the size and shape of the time-activity curve (TAC) in whole brain generated by analyzing PET/CT images co-registered with a mouse brain atlas. Both tracers entered the brain, peaked within first minute of tail vein injection, and cleared gradually over a period of 60 min. ¹¹C-MGX-10S tracer displayed a suitable TAC with an average peak uptake of 5.90±0.89% ID/g (n=4) in contrast to only 1.89±0.35% ID/g (n=4) uptake of ¹¹C-MGX-11S tracer. ¹¹C-MGX-10S was pursued as lead radiotracer for subsequent in vivo studies in the murine model of neuroinflammation: (a) in vivo metabolic stability at 5 min and 20 min, (b) biodistribution of the tracer, (c) PET/CT imaging with and without blocking with GLPG1205, and (d) ex vivo autoradiography with and without blocking with GLPG1205.

The Gpr84 mRNA levels in the brain of the LPS mice with control mice was compared using qRT-PCR. LPS induced ˜17-fold increase of Gpr84 mRNA versus only 5-fold increase of TSPO mRNA. These results motivated testing of the lead radiotracer in the LPS model of neuroinflammation.

### Methods

Animals. All animal procedures were approved by the Stanford Administrative Panel on Laboratory Animal Care (APLAC), accredited by the Association for the Assessment and Accreditation of Laboratory Animal Care (AAALAC International). All federal and state regulations governing the humane care and use of laboratory animals were upheld. For the subsequent experimental procedures, mice were anesthetized using isoflurane gas (2.0-3.0% for induction and 1.5-2.5% for maintenance). Female C57BL/6J wild-type mice were obtained from Jackson Laboratories. All animals were housed in a temperature-controlled environment under a 12-h light/dark schedule with ad libitum food and water access and were acclimatized for 1 week prior to experiments.

Study Design. The main objective of this study was to design and develop ¹¹C-PET tracers to image the expression of GPR-84—a biomarker of neuroinflammation—in myeloid cells. The tracers were first validated in cell culture model using human GPR84-expressing HEK293 cells and THP-1 monocytes. The tracers were next evaluated in an LPS-induced mouse model of sepsis, testing blood brain barrier permeability, in vivo stability, and detection of neuroinflammation. The animals were randomized before imaging. All outliers were included.

Cell culture. Human GPR-84⁺HEK293 cells (Creative Biogene Biotechnology) were cultured in Dulbecco's modified Eagle's medium (DMEM; Life Technologies) containing 10% fetal bovine serum, puromycin (1 μg/mL). THP-1 cells (American Type Culture Collection [ATCC]) were cultured in ATCC-formulated RPMI-1640 Medium (Catalog No. 30-2001) containing 2-mercaptoethanol (final concentration 0.05 mM), and 10% fetal bovine serum. All cells were propagated at 37° C. in a humidified atmosphere containing 5% CO₂. Flp-IN TREx 293 cells expressing a GPR84-Gαi2 fusion protein have been described previously.

Quantitative PCR. Quantitative PCR (qPCR) was performed with SYBR Green Polymerase (Qiagen), RT-PCR Qiagen specified primers (TSPO, GPR84, GAPDH), and cDNA. GAPDH was used as a housekeeping gene for all tissues. Reactions were completed in the Applied Biosystems QuantStudio 6 Real-Time PCR machine. Each sample was run with three technical replicates, and fold change for each gene was calculated by deriving 2^{ddCT} Transcripts with undetectable values were assigned a cycle threshold of 38 for analysis. Samples with high variation between technical replicates (SD>0.70) were excluded from analysis.

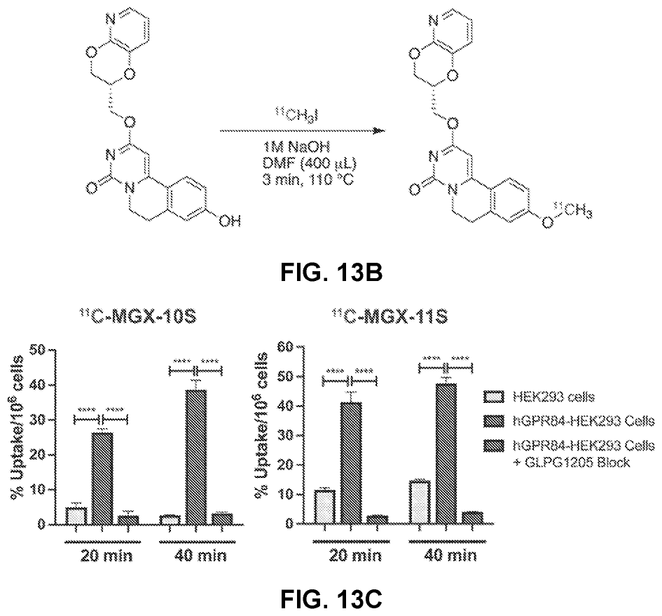

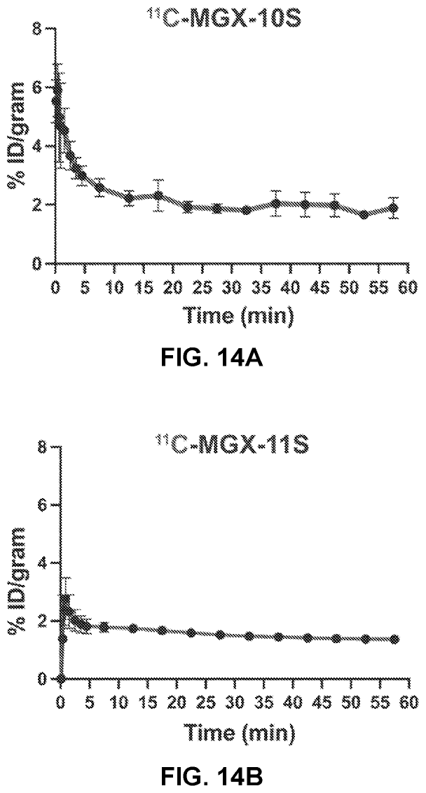

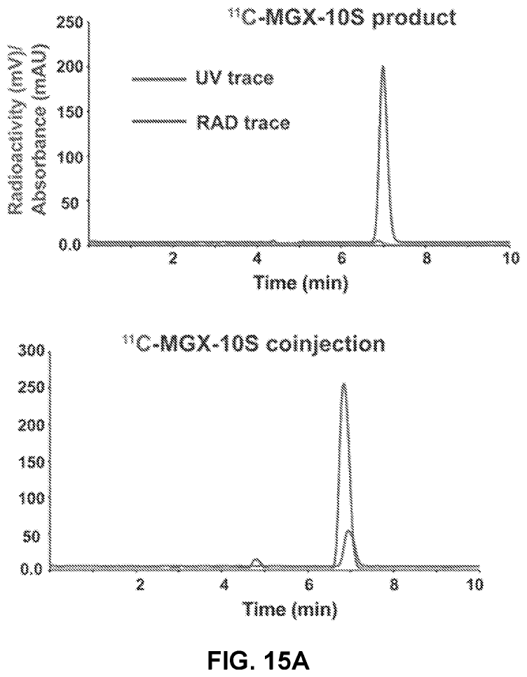

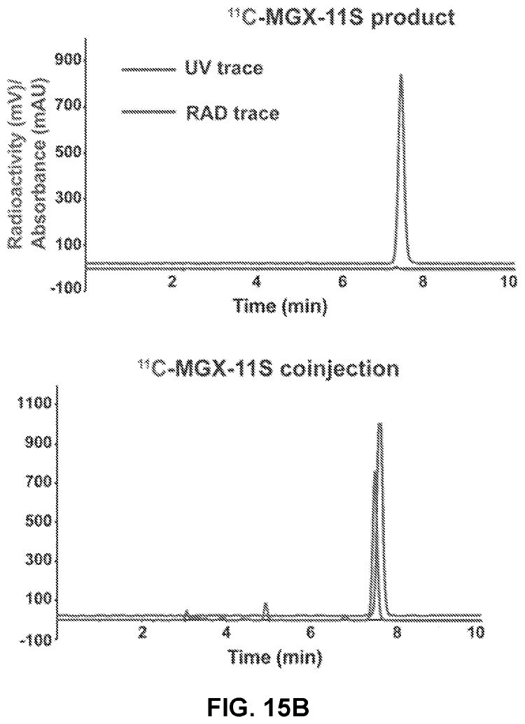

Precursors and cold standards synthesis. First, the cold version of compound MGX-10S was synthesized. MGX-10S is an allosteric inhibitor of GPR84 and determined its kinetic inhibition constant (Kᵢ) via a competition binding assay with ³H-G9543 (known GPR84 inhibitor). HPLC traces of the formulated product alone and coinjected with the cold (non-labeled) standard are shown in FIGS. 14A-14B. ¹¹C-MGX-10S was then generated by alkylating the phenolic precursor with ¹¹C-methyl iodide in DMF (500 μL) for 3 min at 65° C., using 1 M NaOH as a base (FIG. 13A). A similar procedure was followed to synthesize ¹¹C-MGX-11S (FIG. 13B). Subsequently the binding specificity of ¹¹C-MGX-10S and ¹¹C-MGX-11S were evaluated using stable human-GPR84-expressing human endothelial kidney (hGPR84-HEK293) cells versus parental control cells in quadruplicate. Lastly, the in vivo kinetics, distribution, and blood-brain-barrier (BBB) permeability of ¹¹C-MGX-10S in healthy C57BL/6 mice (n=4) were assessed using dynamic PET/CT imaging (FIG. 13C).

The precursors for radiosynthesis were obtained via multi-step organic synthesis illustrated in Scheme 3. The synthesis was modified from previously published work. Briefly, carbamate was produced by heating 2-(3-methoxyphenyl)ethylamine and urea under acidic condition for 2 days. The carbamate was reacted with diethylmalonate to afford hexahydropyrimidine-2,4,6-trione (MGX-2), which was transformed to dihydropyrimido[6,1-a]isoquinolin-4-one (MGX-3) by heating with phosphorus oxychloride (POCl₃) at 55° C. for 2 days. MGX-3 was bifurcated into two parallel routes—the cold standards (MGX-10S and MGX-11S) and the precursors (MGX-8S and MGX-9S). The cold standards were achieved by treating MGX-3 with (R)-enantiomers of dioxane alcohol in the presence of potassium tert-butoxide at 0° C. for 30 minutes, followed by work-up and column purification. Greater than 99% pure products were obtained (assessed via NMR and HPLC) with overall yields of 17.27% (MGX-10S) and 32.55% (MGX-11S) in four step synthesis. Precursors were generated by deprotection of the MGX-3 methoxy group with boron tribromide at −78° C. to room temperature, reprotection with an allyloxy group with ally bromide potassium carbonate at room temperature, dioxane ring installation with dioxane alcohols at 0° C., and finally deprotection of the allyloxy group with palladium (0) catalyst. These steps provided >99% pure precursors (MGX-8S and MGX-9S) (determined via NMR and HLPC) with overall yields of 19.83% (MGX-8S) and 22.28% (MGX-9S).

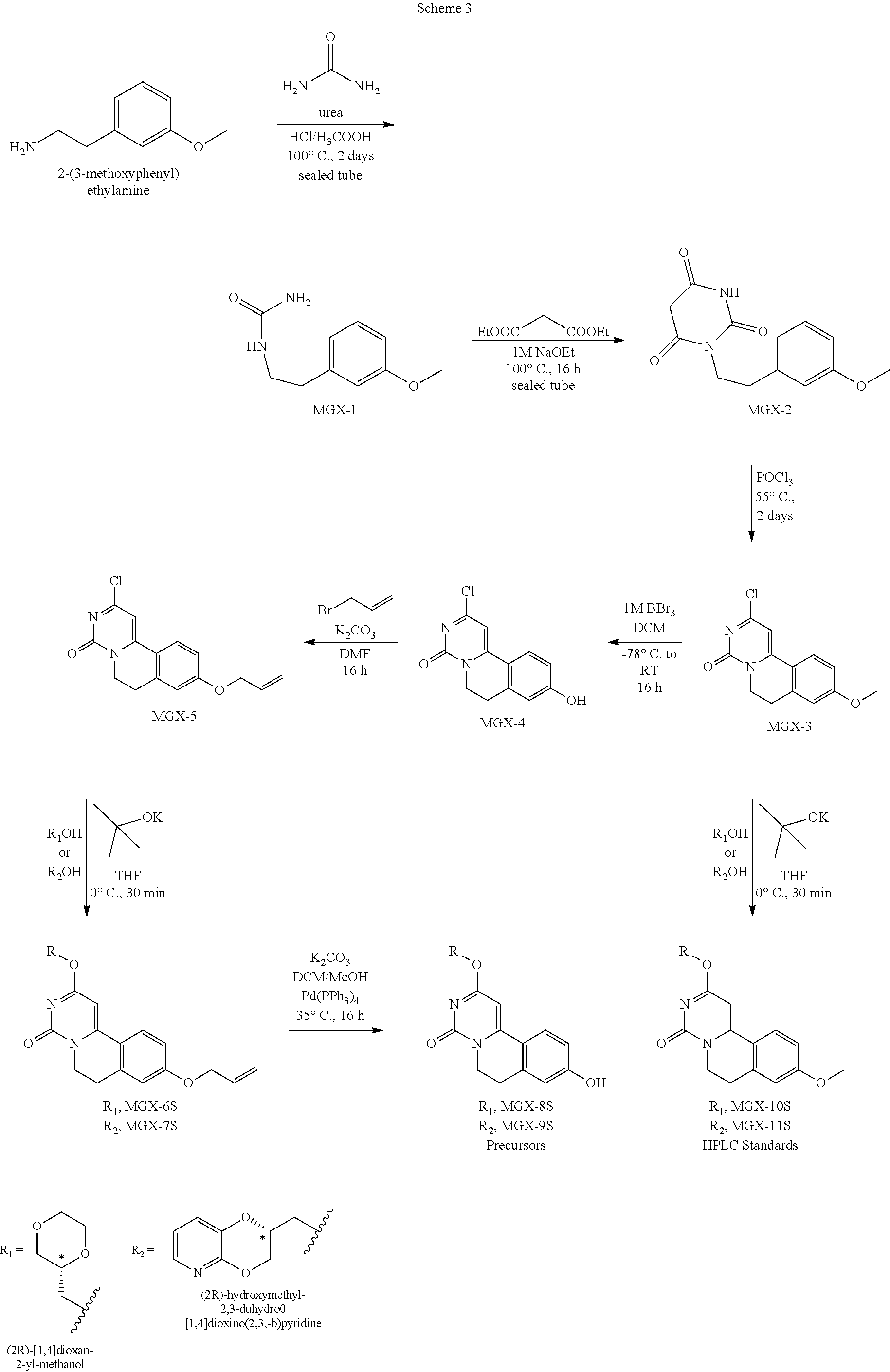

Radiosynthesis. The ¹¹C labeling was first optimized by screening different bases, base molar equivalences, and temperatures. Next, the optimized conditions were applied to synthesize ¹¹C-MGX-10S and ¹¹C-MGX-11S using the GE TRACERLab FX M2 module. Trapped ¹¹CO₂ was converted into ¹¹CH₃I in GE TRACERLab FX Mel, and subsequently delivered to the FX M2 module. For ¹¹C-MGX-10S synthesis, the precursor MGX-7S (0.63 mg, 1.907 μmol) and 1M NaOH (2.28 μL, 1.2 equivalent) base were reacted with [¹¹C]methyl iodide in N, N-dimethylformamide (400 μL) for 5 min at 65° C. For ¹¹C-MGX-11S synthesis, MGX-8S (0.7 mg, 1.78 μmol) and 1M NaOH (2.31 μL, 1.3 equivalent) were treated with [¹¹C]methyl iodide in N, N-dimethylformamide (400 μL) for 3 min at 110° C. The reaction mixture was diluted with 1 mL water and loaded on a semi-prep HPLC for purification Phenomenex Gemini C18 column 5 μm, 110 Å, 250×10 mm), and purified using water+0.1% trifluoroacetic acid/acetonitrile+0.1% trifluoroacetic acid isocratic (70:30, 30 minutes). Each synthesis utilized identical formulation steps. The ¹¹C-MGX-10S (elution time 12.5 min) or ¹¹C-MGX-11S fraction (elution time 15.5 min) was collected into a round bottom flask containing 20 mL water. This was then passed through a preconditioned Sep-Pak C18 Light cartridge (Waters). The loaded cartridge was washed with water (8 mL), and the tracer eluted using ethanol (1 mL) and saline (4 mL). The total synthesis time was 60 min. The radiochemical purity and molar activity were determined via analytical HPLC (Phenomenex Gemini C18 column 5 μm, 110 Å, 250×4.6 mm) using water+0.1% trifluoroacetic acid/acetonitrile+0.1% trifluoroacetic acid isocratic (60:40, 12 minutes). For molar activity calculation, the area under UV absorbance peak of the carrier product at 254 nm was compared to a standard curve of the cold standards (MGX-10S or MGX-11S).

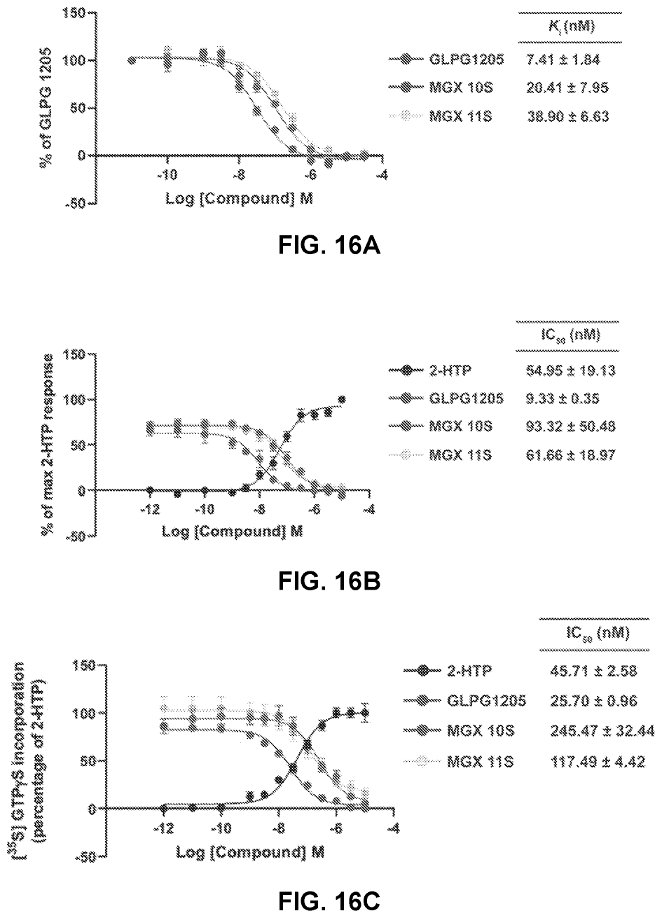

Functional and competition binding assays. GPR84-transduced HEK-293 cells and control HEK-293 cells were plated at a concentration of 3×10⁵ cells per well in media (DMEM with 10% FBS and 1% anti-anti) into three 12-well plates 16-24 hr prior to the cell binding assay. Once cells reached ˜90% confluency, the radiotracer was prepared. Additionally, a blocking solution was prepared of 35 μM GLPG1205 in DMSO, and DMEM media warmed. The radiotracer was diluted in DMEM without FBS or antibiotic to yield a 30 mL solution where the amount of activity was 25 μCi in 500 μL. The first 12-well plate was incubated for 20 minutes at 37° C. and 5% C02 in air while the next was incubated for 40 minutes at the same conditions. Cells were subsequently washed with PBS, trypsinized, resuspended in DMEM+10% FBS, and pipetted into gamma counting tubes at 500 μL volume. Radioactivity levels bound to cells were measured in a gamma counter (Hidex Oy, Helsinki, Finland), and cell counts were recorded using Trypan blue and an automatic cell counter to normalize radioactivity levels to cell count. Results of competition binding assays and functional assays are presented in FIGS. 16A-16C.

Tracer binding assay. hGPR84⁺HEK293 and HEK cells (3×10⁵) were plated into 12-well plates 16 h to 24 h before ¹¹C-MGX-10S or ¹¹C-MGX-11S uptake analysis. On the day of the experiment, fresh, prewarmed DMEM (lacking FBS or antibiotic) containing approximately 0.925 MBq (or 25 μCurie) of ¹¹C-MGX-10S (or ¹¹C-MGX-11S) was added to each well (500 μL per well; μmol). A GLPG1205 blocking solution of 35 μM final concentration was also prepared.

The hGPR84⁺HEK293 (+/−GLPG1205 block) and HEK cells were incubated with ¹¹C-MGX-10S (or ¹¹C-MGX-11S) at 37° C. and 5% CO₂ over a 20 min and 40 min time points (n=4). At the indicated time points, plates were placed on ice, washed three times with ice-cold phosphate-buffered saline (PBS), and trypsinized (150 μL per well) for 2 min at 37° C. The trypsin was neutralized by adding 600 μL DMEM containing 10% FBS (original cell culture media) to each well. The cells from each well (500 μL) were transferred to gamma counting tubes; a tube was also created containing a 500 μL standard of the tracer stock solution (1.85 MBq [or 50 μCurie]/mL). Gamma counting was performed to quantitate percentage radiotracer uptake by the cells and decay-corrected radioactivity was determined (Hidex automated gamma counter). Finally, cells in each gamma counting tube were counted by using the Countess cell counter (Life Technologies). Cell binding was expressed in terms of % uptake/million cells.

In vivo stability assay. Mice were injected with LPS (5 mg/kg) intraperitoneally (i.p.) 24 hours before tracer administration. ¹¹C-MGX-10S was injected via tail vein (650-850 μCi) while mice were under anesthesia. Mice were perfused with PBS buffer (20 mL) under deep anesthesia (5 min, 20 min). The whole brain and liver lobe were removed from mouse place in two separate 50 mL microcentrifuge tubes containing 500 μL acetonitrile, homogenized sample and placed on ice. The well-homogenized samples were transferred to two 1.5 mL microcentrifuge tubes, centrifuged at 9,400 g for 4 min at room temperature. Supernatants were transferred into gamma counting tubes; the pellets were transferred into separate tubes. All supernatants and pellets were counted using gamma counter to measure extraction efficiency. Each of the supernatant (150-200 μL) was transferred into HPLC vials with the inserts and injected into HPLC for radio-metabolic analysis.

In vivo LPS model: imaging studies. All experiments involving animals were in accordance with the Stanford Administrative Panel on Laboratory Animal Care (APLAC), accredited by the Association for the Assessment and Accreditation of Laboratory Animal Care (AAALAC). 3-4 mo female C57BL/6J mice were purchased from Jackson Laboratories (Jax) and were acclimated to the facility for at least 2 weeks prior to experimentation. The mice were in a temperature-controlled environment with a 12 hr light/dark schedule, and provided unrestricted access to food and water.

PET imaging scans were carried out on a docked Siemens Inveon PET/CT scanner (matrix size: 128×128×159; CT attenuation-corrected; nonscatter-corrected) after a bolus intravenous injection of MBq of ¹¹C-MGX-10S into control and LPS mice (μmol). Dynamic brain PET scanning began immediately after administration of ¹¹C-MGX-10S or ¹¹C-MGX-11S and terminated 60 min later. The acquired data were then sorted into 0.5-mm sinogram bins and 19 time frames for image reconstruction (4×15 s, 4×60 s, and 11×300 s), Iterative reconstruction was performed with the following parameters: 3D ordered-subset expectation maximization (3D-OSEM) was followed by fast maximum a posteriori (fastMAP); MAP OSEM iterations, 2; MAP sub-sets, 16; MAP iterations, 18. The count densities were averaged for all volumes of interest at each time point to obtain a time-activity curve (TAC). The TACs were normalized to injected dose, as measured by a CRC-15 PET dose calibrator (Capintec Inc.), and expressed as % injected dose per gram (% ID/g), assuming 1 g/mL. CT was performed in GNEXT depth of interaction (DOI) dual-layer detector system. Scintica VivoQuant software was used to visualize radiotracer uptake and define the 3D volumes of interest. 3D maximum intensity projection (MIP) PET/CT images were created using IRW software.

Ex vivo biodistribution: statistical analysis. Statistical analyses were performed using GraphPad Prism (version 9). Group comparisons were analyzed using two-tailed Mann-Whitney U tests, and multiple comparisons analyzed by one-way ANOVA. Cell binding assays and ex vivo biodistribution studies were analyzed by multiple unpaired Student's t test with Welch's correction. Data are expressed as mean±SEM, unless otherwise indicated; a P value of 0.05 was considered significant.

### Results

Synthesis of MGX-10S and MGX-11S. Previously published methods were modified and optimized to produce cold standards (MGX-10S and MGX-11S) via multi-step organic synthesis. Briefly, 2-(3-methoxyphenyl) ethylamine underwent three step transformations to generate three ring system 9-Methoxy-2-chloro-6,7-dihydropyrimido[6,1-a]isoquinolin-4-one (MGX-3). The resulting MGX-3 product was split into two reaction pathways. Cold standards (MGX-10S and MGX-11S) were produced by treating MGX-3 with the (R)-enantiomers of dioxane alcohol in the presence of potassium tert-butoxide at 0° C. for 30 minutes afterwork-up and column purification. Alternately, the precursors MGX-8S and MGX-9S were produced by a four-step synthesis: boron tribromide induced deprotection of the methoxy group, reprotection of the phenol with allyl group, installation of the dioxane ring, and finally deprotection of the allyl group with [Pd(PPh₃)₄] catalyst.

Optimized synthesis yielded enantiopure precursors and cold standards. In 2020, Labéguère et al. reported a library of GPR84 inhibitors. From this library, two potential molecules were shortlisted for two main reasons: (1) structural amenability for incorporation of a ¹¹C isotope and (2) favorable CNS PET multiparameter optimization (CNS PET MPO) scores indicating their high potential for blood-brain barrier (BBB) permeability. The CNS PET MPO score considers several physicochemical properties including molecular weight (MW), topological polar surface area (TPSA), and lipophilicity (Clog P) of the radioligands. ¹¹C-MGX-10S and ¹¹C-MGX-11S have suitable MW (<400), TPSA (70-82), and Clog D (<2), yielding a cumulative score of 4.8/5.0 for ¹¹C-MGX-10S and 4.1/5.0 for MGX-11S. These scores suggest that these radiotracers have a high likelihood of BBB penetration. In pursuit of cold standards (for quality control using HPLC analysis) and precursors for ¹¹C labeling, a previously reported synthesis was optimized with several modifications as described elsewhere herein. The present synthetic route avoids late stage addition of the methyl group on the phenolic precursor, thereby circumventing the formation of side products via keto-enol tautomerization. Greater than 95% pure products were obtained (assessed via NMR and HPLC) with overall yields of 17.27% (MGX-10S) and 32.55% (MGX-11 S) in four steps. Precursors were generated by deprotection of the MGX-3 methoxy group using boron tribromide at −78° C. to room temperature, followed by reprotection with an allyloxy group using ally bromide/potassium carbonate at room temperature, subsequent dioxane ring installation with dioxane alcohols at 0° C., and finally deprotection of the allyloxy group with palladium (0) catalyst. The resulting precursors, MGX-8S and MGX-9S, were >95% pure (determined via NMR and HLPC) with overall yields of 19.83% (MGX-8S) and 22.28% (MGX-9S).

We also optimized the synthesis of (2R)-hydroxymethyl-2,3-dihydro-[1,4]dioxino[2,3-b]pyridine—an integral part of the ¹¹C-MGX-11S radiotracer. The novel pathway involved a Mitsunobu reaction that transferred enantiomeric purity of the starting glycerol via intramolecular cyclization-induced complete inversion of the chiral center. The previously reported trityl protecting group of the primary alcohol was replaced with tert-butyldiphenysilyl (TBDPS), which is more stable in acidic conditions and protects the scaffold from hydrolysis.

MGX-10S and MGX-11S Bind GPR84 at Nanomolar Concentration. To determine the affinity of the MGX-10S and MGX-11S compounds for human GPR84, competition binding assays were performed using [³H]G9543. Binding of [³H]G9543 to membranes from Flp-In TREx 293 cells expressing a FLAG-human GPR84-Gαi2 fusion protein was fully competed by the compounds MGX-10S and MGX-11S with Kᵢ in the nanomolar range; this was similar to that observed for the reference GLPG1205 (Kᵢ=24.53±14.26 nM, Kᵢ=39.92±12.83 nM and Kᵢ=7.99±3.40 nM, respectively; FIG. 16A). Compounds MGX-10S and MGX-11S were also fully blocked in a concentration-dependent manner, and inhibition of forskolin-amplified cAMP levels was carried out by 2-HTP in the same cells (IC₅₀=104.70±58.79 and 70.28±36.28 nM, respectively; FIG. 16B). This membrane system was finally used to demonstrate that MGX-10S and MGX-11S were each able, in a concentration-dependent manner, to fully block activation of human GPR84 promoted by 2-(hexylthiol)-pyrimidine-4,6 diol (2-HTP) in [³⁵S] GTPγS binding assays with potency in the high nanomolar range (IC₅₀=250.40±39.07 and 116.90±6.33 nM, respectively; FIG. 16C). These two functional assays confirmed that MGX-10S and MGX-11 S are antagonists with high nanomolar IC₅₀. The high potency (low nanomolar binding affinity Kᵢ) combined with moderate inhibitory effects (high nanomolar IC₅₀ values in the functional assays) predicts ¹¹C-MGX-10S and ¹¹C-MGX-11S as potential “Goldilocks” PET tracers.

Radiochemistry. The phenolic precursors MGX-8S and MGX-9S (Scheme 3) were radiolabeled with [¹¹C]methyliodide (¹¹CH₃I) in presence of 1M NaOH base. The ¹¹C-MGX-10S and ¹¹C-MGX-11S radiotracers were produced via nucleophilic substitution reaction using the GE TRACERLab FX M2 module: ¹¹C-MGX-10S was synthesized in DMF (400 μL) at 65° C. for 3 min, while ¹¹C-MGX-11S was synthesized in DMF (400 μL) at 110° C. for 3 min. Semi-preparative reverse-phase high performance liquid chromatography (HPLC) of the crude reaction mixture afforded ¹¹C-MGX-10S or ¹¹C-MGX-11S at a purity of >99% with high molar activity (Table 1). The formulated ¹¹C-MGX-10S or ¹¹C-MGX-11S in saline/ethanol (v/v 9:1, total 10 mL) was stable for at least 4 h via analytical reverse-phase HPLC.

TABLE 1
Quality Control Summary of ¹¹C Radiotracers

| | Radiochemical | Radio- | |
|---|---|---|---|
| | Yield (decay | chemical | Molar Activity |
| Tracers | uncorrected) (%) | Purity (%) | (mCi/μmol) |
| ¹¹C-MGX-10S | 1.30 ± 0.40 (n = 12) | >99 | 17484.13 ± 6577.08 |
| ¹¹C-MGX-11S | 1.16 ± 0.15 (n = 3) | >99 | 4116.03 ± 2335.71 |

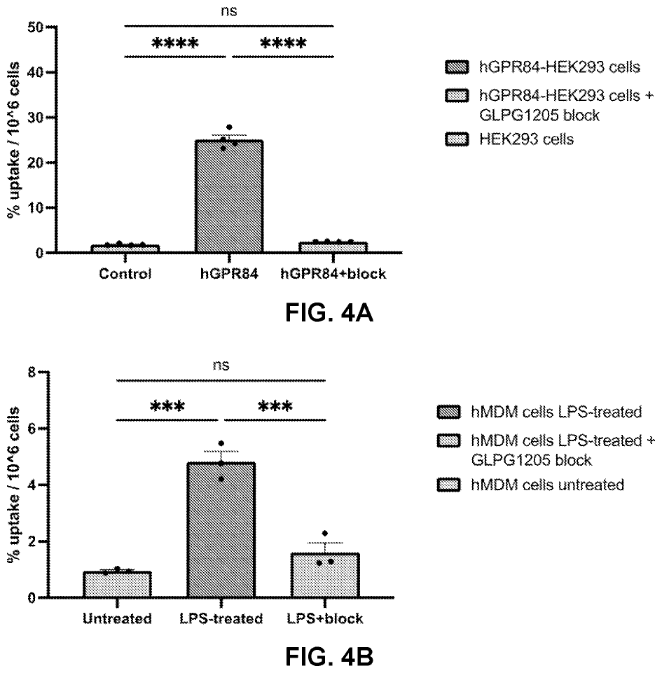

¹¹C-MGX-10S and ¹¹C-MGX-11S specifically bind human GPR84 in cells. With two new candidate GPR84 radiotracers in hand, their binding in cells was assessed with and without the expression of human GPR84. Cell binding studies revealed 14.5-fold higher binding of ¹¹C-MGX-10S to hGPR84⁺HEK293 cells compared to parental HEK293 cells after 40 min of incubation (n=4, P<0.0001). A similar pattern was observed for radiotracer binding after 20 min incubation (FIGS. 4A-4B. Co-incubation of ¹¹C-MGX-10S with the GPR84 antagonist GLPG1205 (35 μM) for 40 min significantly reduced radiotracer binding to hGPR84⁺HEK293 cells by 91.7%, demonstrating its high specificity (n=4, P<0.0001). In contrast, ¹¹C-MGX-11S bound hGPR84+HEK293 cells with only a moderate 3.25-fold increase compared to the parental cells after 40 min of incubation (P<0.0001, n=4). The higher target to background binding of ¹¹C-MGX-10S compared to ¹¹C-MGX-11S could be due to the higher lipophilicity of ¹¹C-MGX-11S (calculated clog P=0.963 vs clog P=0.313 for ¹¹C-MGX-10S), which can lead to some potential nonspecific binding or it could be due to the higher affinity of ¹¹C-MGX-10S, resulting in higher overall binding to cells expressing hGPR84 compared to ¹¹C-MGX-11S. Co-incubation with GLPG1205 (35 μM) decreased ¹¹C-MGX-11S binding by 91.5% (P<0.0001, n=4), indicating that while the overall binding is lower than ¹¹C-MGX-10S, it is still specific for GPR84.

¹¹C-MGX-10S and ¹¹C-MGX-11S cross the blood-brain barrier of healthy mice. After evaluating the candidate GPR84 radiotracers in vitro, their ability to cross the blood-brain barrier was compared in healthy mice, finding the initial brain uptake (1 min postinjection) of ¹¹C-MGX-10S to be higher than that of ¹¹C-MGX-11S (FIGS. 14A and 14B). Co-registration of PET/CT images with a mouse brain atlas and subsequent quantification of the radioactive signal in the whole brain over time demonstrated rapid entry of ¹¹C-MGX-10S into the brain with an average peak uptake of 5.90±0.89% ID/g (n=4) within 22.5 s, decreasing to 1.89±0.35% ID/g by the end of the 60 min scan, demonstrating favorable brain uptake with fast washout rate (Brain 1 min/60 min>3; illustrating that the uptake of ¹¹C-MGX-10S in healthy mouse brain peaked within 1 min and that less than ⅓rd of the initial radiotracer activity remained at 60 min). Whole body 3D maximum intensity projection (MIP) PET/CT images using ¹¹C-MGX-10S illustrate a robust brain signal at 0-2 min with increasing renal/hepatic clearance, in addition to a low background signal in all other areas of the body, by the later time point (30-60 min). In contrast, ¹¹C-MGX-11S displayed a lower and slightly slower peak brain uptake of 2.77±0.71% ID/g within 52.5 s, which gradually decreased over the duration of the scan to 1.36±0.14% ID/g with a slower brain washout rate (Brain 1 min/60 min˜2) than ¹¹C-MGX-10S. It was determined that ¹¹C-MGX-10S has a more favorable kinetic profile for imaging GPR84 in the CNS than ¹¹C-MGX-11S and that its ratio of peak uptake-to-levels at the end of the scan is comparable to, if not more favorable than, two other small molecule PET tracers used for clinical neuroimaging research studies, namely, ^{11C}-UCBJ and ¹¹C-Raclopride. ¹¹C-UCBJ, a radiotracer that binds the synaptic vesicle glycoprotein 2A receptor and is used to image synaptic density, has a ratio of 3 in healthy mouse brain scans [the radiotracer reached maximum uptake at 15 min post-tracer injection and gradually cleared over a span of 90 min, with brain pharmacokinetics (Brain 15 min/90 min˜3)]. Another widely used CNS PET radiotracer, ¹¹C-Raclopride, known to bind postsynaptic dopamine D2 receptors, achieved maximum uptake in 5 min in healthy rodents and gradually reached a plateau over 60 min with washout pharmacokinetics of Brain 5 min/60 min˜2.

In summary, the favorable uptake and washout pharmaco-kinetics of ¹¹C-MGX-10S radiotracer in healthy mice (Brain 1 min/60 min>3) warranted further investigation of this radiotracer in a murine model of innate immune activation. As part of these studies, in vivo metabolic stability, biodistribution (via gamma counting), and in vivo specificity of ¹¹C-MGX-10S in the brain were assessed via PET/CT imaging with and without blocking.

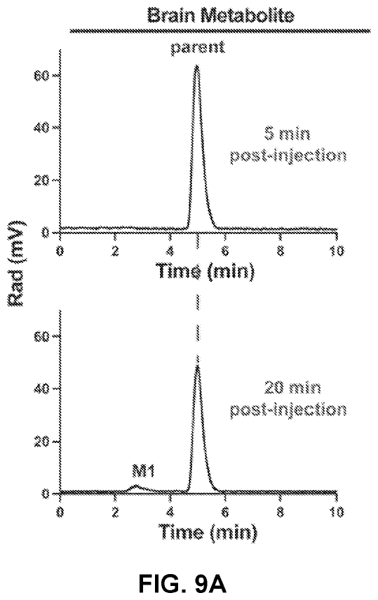

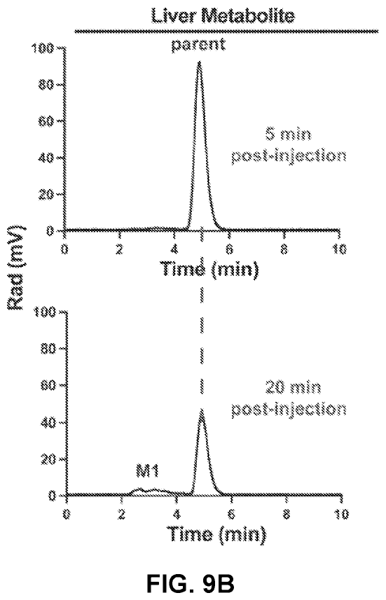

Ex Vivo autoradiography shows specific binding of ¹¹C-MGX-10S tracer in brain. To investigate the metabolic stability of ¹¹C-MGX-10S in vivo in the presence of inflammation, saline (group 1) and LPS [5 mg/kg, intraperitoneal (i.p.), group 2] were administered to C57BL/6 mice 24 h before radiotracer injection. At 5 and 20 min post-tracer injection, the analysis of the whole brain and liver was performed using analytical HPLC to assess the presence of potential radiometabolites. In saline-treated mice, ¹¹C-MGX-10S was largely intact in the brain and liver at 5 min postinjection, with one polar metabolite, M1 peak (HPLC elution time=2.8 min) in the plasma at this time. The tracer underwent low to moderate metabolism in the healthy brain, liver, and plasma 20 min postinjection, as reflected in the M1/parental peaks ratios. These analyses of LPS-treated mice revealed 100 and 97% intact radiotracer (parent radiotracer HPLC elution time=5 min) in the brain at 5 min (n=3) and 20 min (n=3) post-tracer injection, respectively (FIG. 9A). HPLC data of LPS mice plasma at 5 min postinjection showed the major parental peak while samples analyzed at 20 min postinjection revealed one metabolite M1 in addition to the parental peak. The inflamed mouse liver metabolized the radiotracer to some extent as the percentage of the intact radiotracer dropped from 100% to 90% within 20 min post-tracer injection (FIG. 9B). The action of cytochrome P450 (CYP)-3A4 and CYP2C19 enzymes in the liver likely resulted in the minor (10%) hydrophilic radiometabolite M1 (elution time=2.8 min). When compared between mouse and human liver microsomes and hepatocytes, unlabeled compound MGX-10S displayed 10-fold and 22-fold higher stability, respectively. Based on these data, it predicted that the intact ¹¹C-MGX-10S radiotracer will persist longer in the human brain relative to the mouse brain.

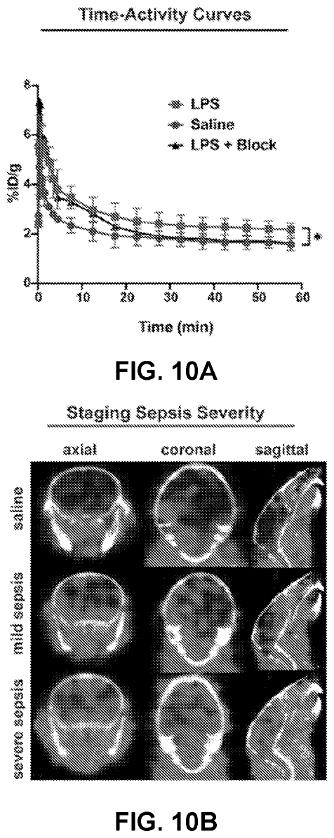

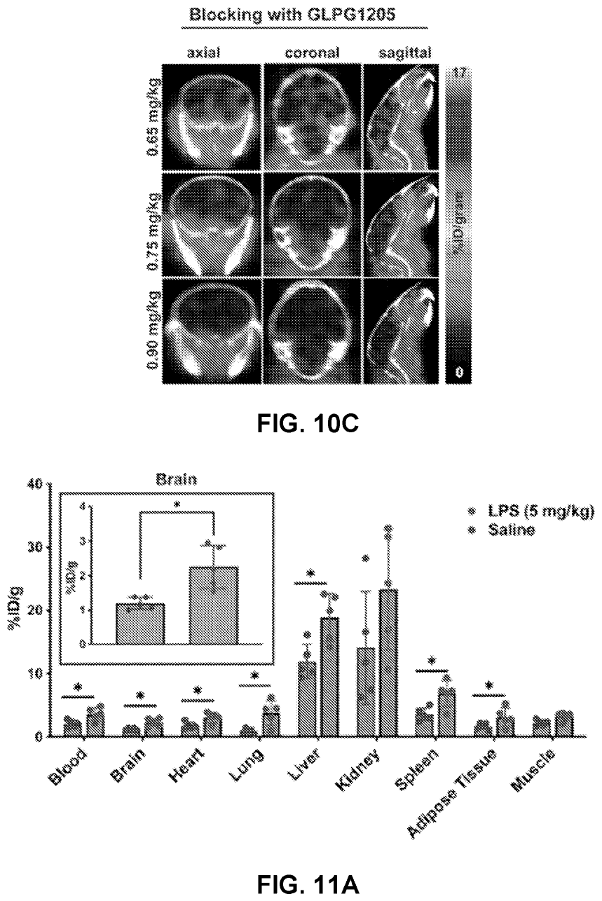

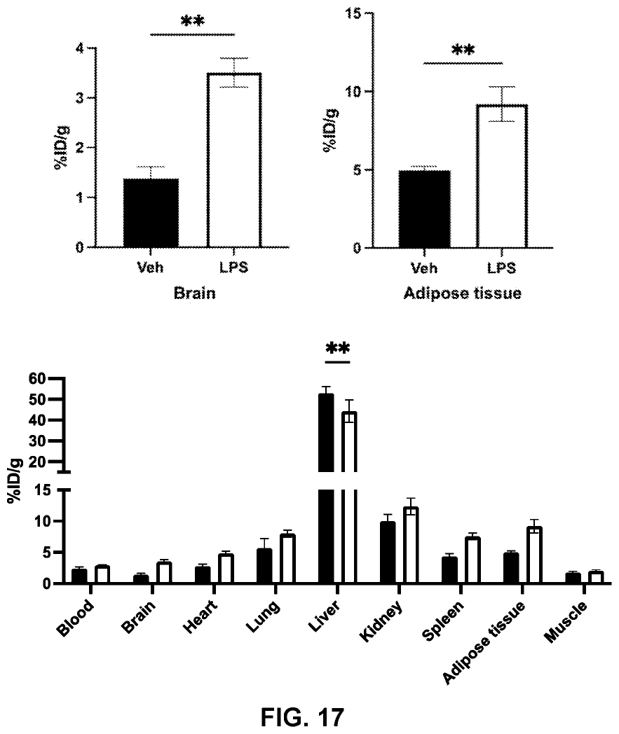

¹¹C-MGX-10S accumulates significantly higher in the LPS brain than in the control brain, while ¹¹C-MGX-10S radiometabolites are absent in the brain or liver. Administration of LPS intraperitoneally to mice is known to trigger rapid systemic inflammation in multiple peripheral organs, in addition to neuroinflammation as soon as 3 h after injection which can last up to 10 months, as evidenced by elevated levels of proinflammatory cytotoxic tumor necrosis factor alpha (TNFα) and increased Iba1-positive immunostaining. This rodent model of systemic inflammation has been used extensively to evaluate the ability of new radiotracers to accurately detect and quantify innate immune activation. Most recently, Horti et al. reported a ¹¹C-CPPC radiotracer targeting CSF1R (a candidate biomarker of innate immune cell-driven inflammation) to have 55% higher binding in the mouse brain when injected with LPS (10 mg/kg) compared to a control animal. This radiotracer displayed significant nonspecific binding since the CSF1R knockout mouse still showed an extensive signal. Another example is the ¹¹C-GSK1482160 radiotracer, which binds purinergic receptor subtype 7 (P2×7)—expressed on activated microglia induced by LPS (5 mg/kg) at a 3.2-fold higher amount than binding in saline-treated mice brains. Although this is a promising fold difference in binding, P2×7 is a suboptimal biomarker for innate immune activation since it is not solely expressed on myeloid cells. In the current work, the pharmacokinetics of ¹¹C-MGX-10S were characterized in female C57BL/6J mice. with and without LPS (5 mg/kg) treatment. Whole-body 3D MIP PET/CT images revealed a marked accumulation of ¹¹C-MGX-10S in the brain, large intestine, liver, and stomach of the LPS-treated mice. The brain was next focused on to assess the ability of ¹¹C-MGX-10S to detect neuroinflammation associated with this murine model. Whole brain time-activity curves (TACs) demonstrated rapid BBB penetration of the radiotracer in LPS-treated mice within 2 min and a sustained higher uptake compared to saline-treated mice (1.40-fold higher accumulation at 20 min and 1.42-fold higher by the end of the scan, P<0.05) (FIG. 10A). The 30-60 min integration of the whole brain PET signals of saline- and LPS-treated mice resulted marked increase in radiotracer uptake (approximately 39.27%, P<0.01) in the LPS brain. All areas of the LPS-treated mouse brain displayed a significantly higher signal (except for the cerebellum), suggesting upregulation of GPR84 expressing immune cells in these tissues. Notably, ¹¹C-MGX-10S PET images clearly enable delineation of different disease severities [i.e., LPS score 0.5 (mild disease) versus 1.5 (severe disease)](FIG. 10B) and show dose-dependent blocking (FIG. 10C). Biodistribution of ¹¹C-MGX-10S is also shown in FIG. 17.

¹¹C-MGX-10S Specifically Binds GPR84 in vivo in the Context of Inflammation. locking studies were performed using the GLPG1205—GPR84 antagonist being evaluated in clinical trials. A range of doses (0.65, 0.75, and 0.90 mg/kg) of GLPG1205 were used to investigate whether the blocking was dose-dependent. For this study, LPS (5 mg/kg, i.p.) was injected into mice (n=6) 24 h before radiotracer injection. GLPG1205 was injected intra-venously 10 min prior to radiotracer injection (n=4). Preblocking with GLPG1205 markedly reduced whole-body radiotracer binding compared to mice administered LPS alone. The blocking agent (0.65 mg/kg) attenuated the PET signal in the LPS-treated mice brain by 38.50% at the end of the scan (FIG. 10B). This reduction in the signal after GLPG1205 treatment is shown in the 30-60 min summed brain PET images, which illustrate a decrease in radiotracer binding almost to the level of the saline-treated mice brain. Dose-dependent GLPG1205 reductions in the brain PET signal were also observed (FIG. 10C). This study clearly demonstrates the specific binding of ¹¹C-MGX-10S to GPR84 in the mouse brain in vivo. Overall, these data suggest that ¹¹C-MGX-10S has great promise for studying GPR84 expression in the brain in the context of inflammation.

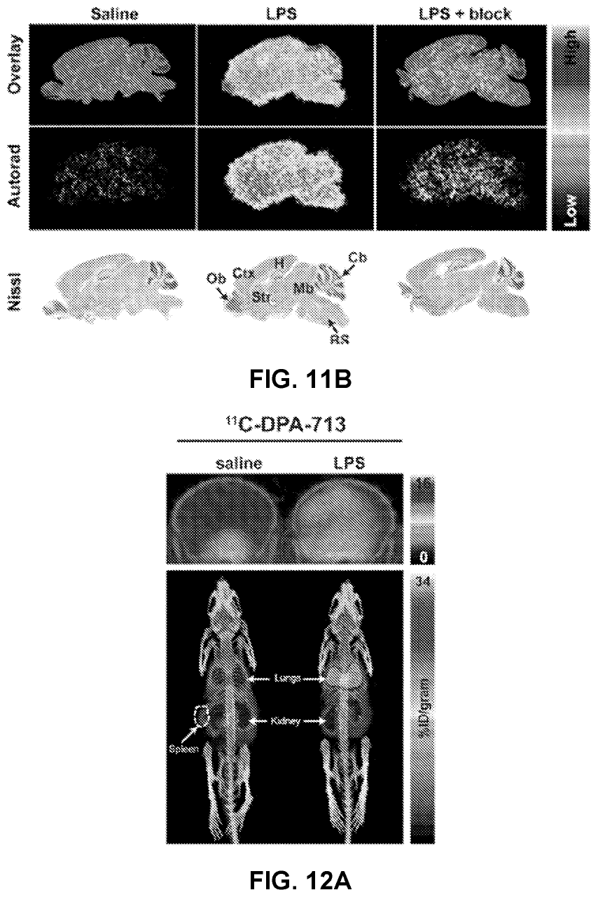

Ex vivo Biodistribution Demonstrates Higher Radiotracer Accumulation in the LPS Brain. Whole-brain TACs suggest that ¹¹C-MGX-10S binding in LPS mice begins to stabilize at −20 min post-tracer injection. Due to the short half-life of the ¹¹C radiotracer (T_{1/2}=20 min) and its initial stable pharmacokinetics (Brain20 min/60 min˜1.2), ex vivo biodistribution was assessed at 20 min postinjection of the radiotracer. TNFα production in the liver and blood of these mice ultimately triggers brain microglial activation, prompting proinflammatory protein synthesis.14 These neuroinflammatory events portend high accumulation of ¹¹C-MGX-10S in the blood, liver, and brain due to severe inflammation. Indeed, increased accumulation of ¹¹C-MGX-10S was observed in several tissues from LPS-treated mice (including adipose tissue, blood, brain, heart, liver, lung, and spleen) compared to that in saline-treated mice. Specifically, >1.8-fold increased radiotracer uptake was observed in the brain (0.84±0.21% ID/gram) and adipose tissue (1.16±0.50% ID/g) of LPS-induced mice compared to those treated with saline (0.45±0.06 and 0.63±0.15% ID/g, respectively), consistent with known GPR84-mediated inflammatory processes in these tissues following LPS challenge. The highest signal for both LPS- and saline-treated mice was observed in metabolic and excretory organs, including the liver (LPS: 7.08±1.27% ID/gram, saline: 4.51±0.15% ID/gram, P<0.05) and kidneys (LPS: 8.73±3.18% ID/gram, saline: 5.32±3.01% ID/gram P=not significant) indicative of a clearance pattern consistent with the kinetics of most small molecule radiotracers (FIG. 11A). The significant radiotracer accumulation in the liver likely represents LPS-induced inflammation in combination with radiotracer clearance.

Our results align with previous reports detailing elevated gpr84 mRNA expression in adipose tissue, brain, and lung from mice treated with LPS (1 mg/kg). While this prior study did not use the same amount of LPS as in the present study, nor did it investigate all the tissues examined herein, it serves to affirm, at least in part, what was observed in this study. Bouchard and colleagues found a 4-fold increase in the brain gpr84 at 24 h post-LPS compared to saline-injected mice. They applied in situ hybridization to show that gpr84 mRNA expression was restricted to the leptomeninges and blood vessels 3 h after LPS treatment but was widespread throughout the brain at 24 h. This effect was absent following GPR84 antagonist treatments that partially block AKT and ERK phosphorylation and expression of proinflammatory cytokines (interleukin [IL]-6, IL-12b, and tumor necrosis factor [TNF]-α), suggesting that pro-inflammatory cytokines likely regulate the endotoxin-induced GPR84 expression in the cerebral cortex of mice.

Ex vivo Audoradiography Confirms ¹¹C-MGX-10S Specificity. To obtain a high-resolution view of radiotracer distribution in the brain, ex vivo autoradiography was performed 20 min after ¹¹C-MGX-10S injection. Brains from LPS-treated mice showed widespread radiotracer binding (matching previously published data on spatial gpr84 expression in the brain of LPS-treated mice at this time point), with slightly higher intensity in the striatum (FIG. 11B). Preblocking with GLPG1205 (1 mg/kg) produced a dramatic reduction in the binding of ¹¹C-MGX-10S, approximately to the level of the saline-treated mouse brain (FIG. 11B). These results confirm the specificity of ¹¹C-MGX-10S for GPR84 in the mouse brain.

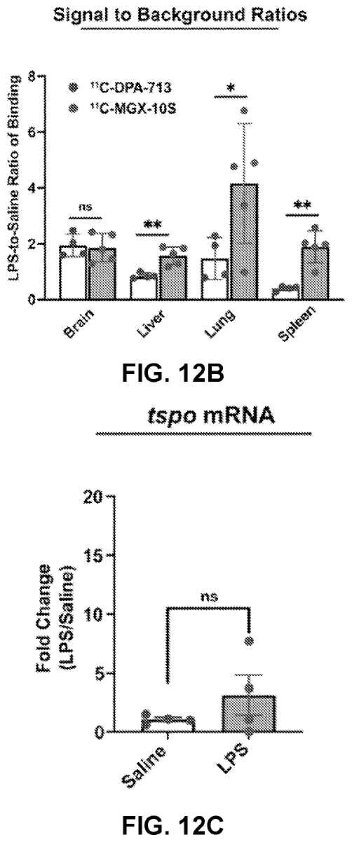

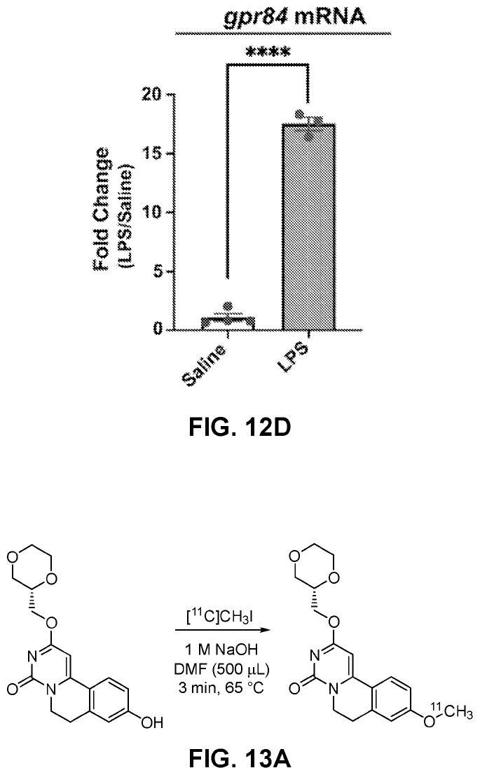

GPR84 PET is More Sensitive than TSPO PET. Next ¹¹C-MGX-10S was compared with an existing clinical research radiotracer [¹¹C]N,N-Diethyl-2-[2-(4-methoxyphen-yl)-5,7-dimethylpyrazolo[1,5-a]pyrimidin-3-yl]acetamide (¹¹C-DPA-713) that targets TSPO—a widely evaluated imaging biomarker of neuroinflammation.37 In saline-treated mice, ¹¹C-DPA-713 concentrates in the kidneys, lungs, and spleen (all known to be TSPO-rich tissues). Unfortunately, the high TSPO-PET signal in many healthy tissues can make detection of early alterations in this target in the context of disease challenging and can obscure one's ability to visualize changes even under the most severe inflammatory conditions. For example, changes in the spleen signal in LPS-treated mice are difficult to delineate in TSPO-PET images due to the neighboring intense kidney signal (FIG. 12A). To ascertain the sensitivity of ¹¹C-DPA-713 compared to the disclosed new GPR84 radiotracer, the signal-to-background ratios were directly compared using biodistribution data (expressed as a ratio of the signal in LPS to that in saline-treated mouse organs). While these data revealed similar brain uptake ratios for both radiotracers (¹¹C-MGX-10S: 1.86, ¹¹C-DPA-713: 1.94, P=not significant), ¹¹C-MGX-10S afforded higher signal-to-background ratios than ¹¹C-DPA-713 in multiple inflamed peripheral organs including the liver (1.57 vs 0.86, P<0.005), lungs (4.16 vs 1.48, P<0.05), and spleen (1.89 vs 0.40, P<0.005) (FIG. 12B), signifying its ability to detect innate immune activation with higher sensitivity in these tissues. To investigate the reason for this observed higher sensitivity of the disclosed GPR84 radiotracer, the change in gpr84 and tspo gene expression in the brains from saline- and LPS-treated (5 mg/kg) mice was characterized. Using qRT-PCR, it was found that gpr84 was upregulated to a greater extent than tspo (approximately 17-fold change for gpr84 versus 3-fold for tspo) in the brains of LPS-stimulated mice. (FIGS. 12C-12D). These results support the high potential of GPR84 imaging to enable the sensitive detection of innate immune activation in neuroinflammatory diseases.

Results summary. The 1,4-dioxane ring linked compound (MGX-10S) was shown to have a Kᵢ of 8.22 nM in the competitive binding assay. Automated module synthesis of ¹¹C-MGX-10S was completed in 60 minutes, with a radiochemical yield of 5.07±1.42% (non-decay corrected), radiochemical purity >99%, and molar activity of 6328±396 mCi/μmol. Cell binding studies revealed 14.5-fold higher binding of ¹¹C-MGX-10S to hGPR84-HEK293 cells compared to parental HEK293 cells after 40 minutes of incubation (P<0.0001, n=4). Blocking with GPR84 antagonist (GLPG1205, 35 μM), significantly reduced tracer binding to hGPR84-HEK293 cells by 91.7%, demonstrating high specificity of ¹¹C-MGX-10S (P<0.0001, n=4). A similar pattern of binding was observed for tracer binding after 20 minutes incubation (FIG. 13B). Whole brain time-activity curves (FIGS. 14A and 14B), generated by analyzing PET/CT images co-registered with a mouse brain atlas, demonstrate rapid entry of ¹¹C-MGX-10S into the brain with an average peak uptake of 5.90±0.89% ID/g (n=4) within 22.5 seconds, decreasing to 1.89±0.35% ID/g by the end of the 60 minute scan. Whole body 3D maximum intensity projection PET/CT images illustrate robust brain signal at 0-5 min with increasing renal/hepatic clearance, in addition to low background signal in all other areas of the body, by the later time point (30-60 min).

### Conclusion

A novel PET tracer for the measurement of GPR84 expression has been successfully synthesized and is currently being further evaluated. The data presented herein demonstrate the promise of ¹¹C-MGX-10S as a highly specific tracer for detecting GPR84-expressing myeloid cells in vitro and in vivo. Further studies are currently underway to explore the utility of ¹¹C-MGX-10S for imaging GPR84-associated innate immune activation in rodent models of CNS diseases.

Herein, a proinflammatory orphan receptor known as GPR84 was identified as a promising biomarker of detrimental innate immune activation. To image GPR84 and assess its utility as a PET biomarker, two novel ¹¹C radiotracers were synthesized with high radiochemical yields and molar activity and evaluated their specificity in vitro and blood-brain barrier permeability in vivo. Based on the observed favorable brain uptake and pharmacokinetics of ¹¹C-MGX-10S, this radiotracer was selected as the lead imaging agent to measure GPR84 expression associated with inflammation in a mouse model of systemic and neuro-inflammation. The disclosed radiotracer displayed significantly higher binding in the brain and liver of mice treated with LPS (among other tissues), compared to mice treated with saline. Robust reduction in the PET signal was observed when mice were pretreated with GLPG1205 antagonist, affirming the specificity of ¹¹C-MGX-10S for GPR84 in the context of inflammation. Importantly, these results using sensitive ex vivo techniques, including gamma counting and autoradiography, corroborated the in vivo PET imaging findings described elsewhere herein. Subsequently, the new GPR84-targeting radiotracer was compared with a widely studied TSPO-targeting radiotracer, ¹¹C-DPA-713, and demonstrated the superior signal-to-background ratio of ¹¹C-MGX-10S for identifying regions containing inflammation. Taken together, this work resulted in a new highly specific imaging tool for quantifying GPR84 in vitro and in vivo, and the data using this radiotracer underscore the high potential of GPR84-PET versus TSPO-PET for sensitive and specific detection of innate immune activation. These studies have laid the foundation for future work involving other murine models of inflammatory diseases and human post-mortem tissues to further assess the promise of this radiotracer and GPR84 PET imaging for eventual clinical use.

### Example 3: ¹⁸F PET Tracers to Image GPR84 Expression During Neuroinflammation

In addition to ¹¹C labeled tracers, several ¹⁸F tracers have been developed. Scheme 4 shows a synthetic mechanism for generating ¹⁹F standards for comparison with ¹⁸F tracers.

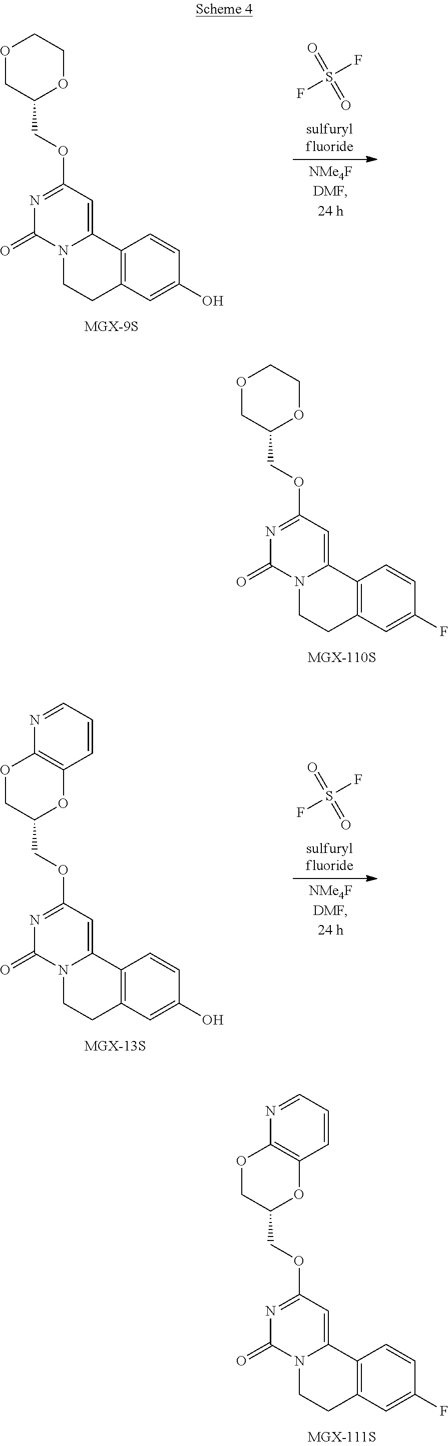 [SMILES: O=c1nc(OC[C@@H]2COCCO2)cc2n1CCc1cc(O)ccc1-2 ; O=S(=O)(F)F ; O=c1nc(OC[C@@H]2COCCO2)cc2n1CCc1cc(F)ccc1-2 ; O=c1nc(OC[C@@H]2COc3ncccc3O2)cc2n1CCc1cc(O)ccc1-2 ; O=S(=O)(F)F ; O=c1nc(OC[C@@H]2COc3ncccc3O2)cc2n1CCc1cc(F)ccc1-2]

Scheme 5, meanwhile, shows radiosynthesis of PET tracers via ¹⁸F labeling:

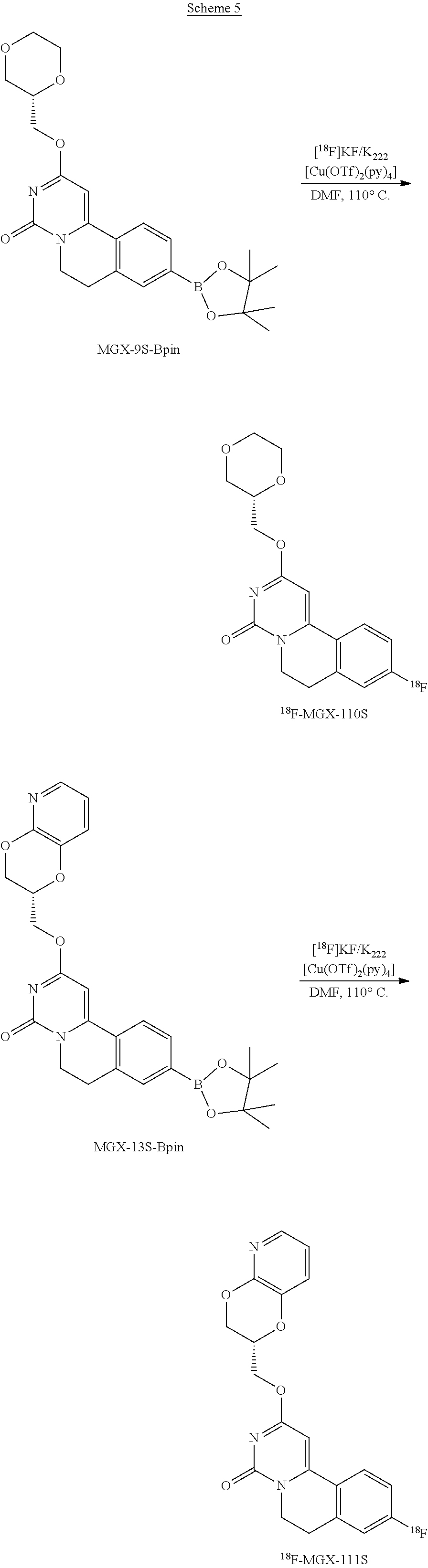 [SMILES: CC1(C)OB(c2ccc3c(c2)CCn2c-3cc(OC[C@@H]3COCCO3)nc2=O)OC1(C)C ; O=c1nc(OC[C@@H]2COCCO2)cc2n1CCc1cc([18F])ccc1-2 ; CC1(C)OB(c2ccc3c(c2)CCn2c-3cc(OC[C@@H]3COc4ncccc4O3)nc2=O)OC1(C)C ; O=c1nc(OC[C@@H]2COc3ncccc3O2)cc2n1CCc1cc([18F])ccc1-2]

### Example 4: Synthesis, In Vitro, and In Vivo Data for Selected ¹⁸F Compounds

### ¹⁸F-MGX-110S and Related Compounds

Synthesis of the Bpin Precursors, MGX-90 and MGX-100 is presented in Scheme 6.

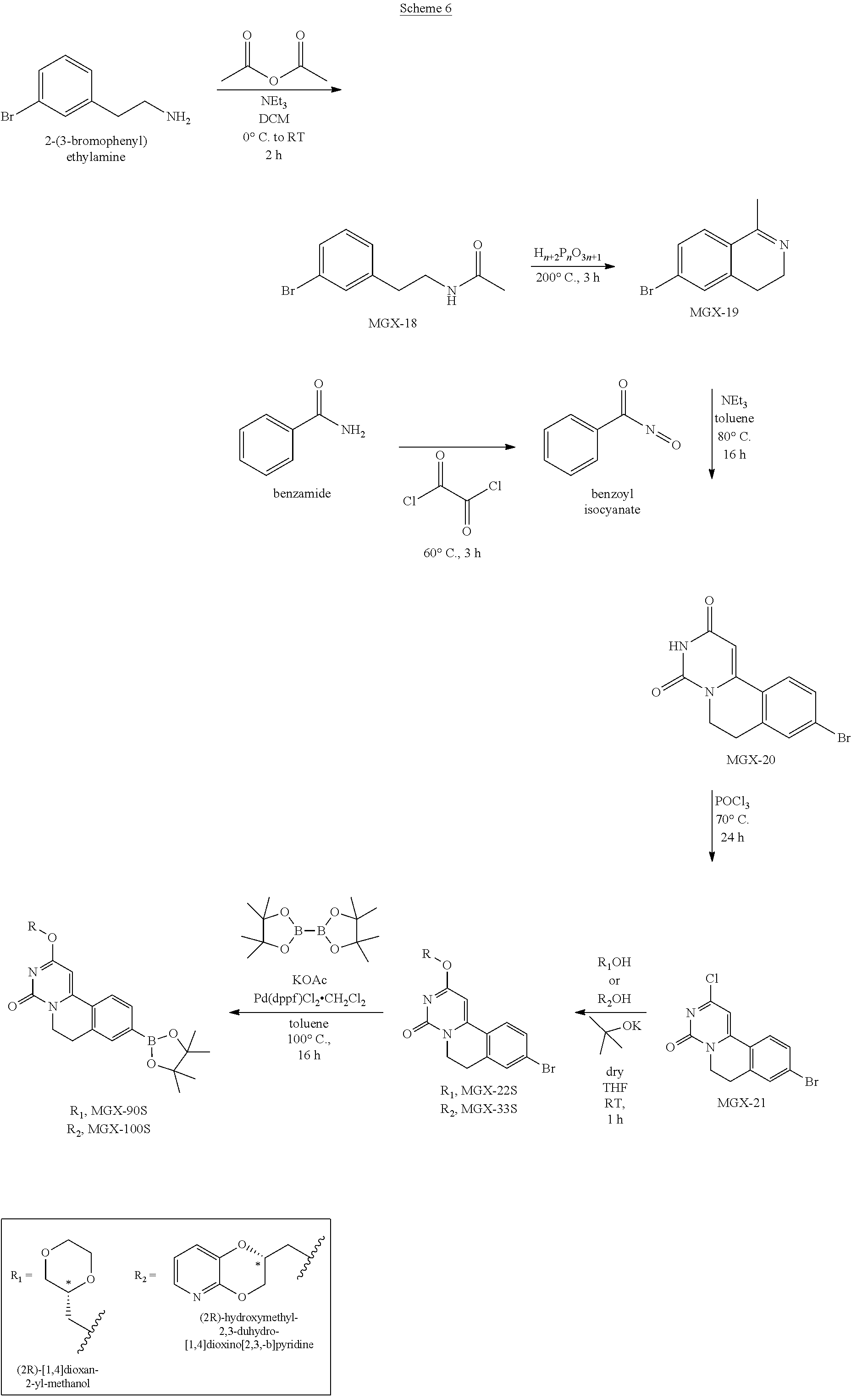

### N-(3-Bromophenethyl) acetamide (MGX-18)

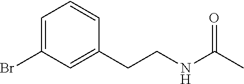

TEA (2.12 g, 21 mmol) was added to a solution of 2-(3-bromophenyl)ethylamine (2.0 g, 10 mmol) in DCM (30 mL). After acetic anhydride was added dropwise at 0° C., the reaction mixture was stirred at room temperature for 3 hours and monitored by TLC. At completion, the reaction was washed with 10% aqueous NaCl, dried over Na₂SO₄, filtered, and concentrated under vacuum, giving 2.48 g (quantitative yield) of N-(3-bromophenethyl)acetamide as a yellowish oil. ¹H-NMR δ (CDCl₃): 7.36 (m, 2H), 7.18 (dt, J=7.6, 0.6 Hz, 1H), 7.12 (m, 1H), 5.54 (bs, 1H), 3.49 (dt, J=7.0, 6.9 Hz, 2H), 2.79 (t, J=7.0 Hz, 2H), 1.95 ppm (s, 3H).

### 6-Bromo-1-methyl-3,4-dihydroisoquinoline (MGX-19)

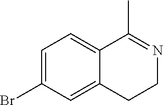

N-(3-bromophenethyl)acetamide (2.48 g) was treated with polyphosphoric acid (8.43 g) and heated to 200° C. for 4 hours. The reaction mixture was poured into stirred ice-cold water (150 mL), the pH adjusted with 30% aqueous NH₄OH to 10 and extracted with ethyl acetate (3×50 mL). The organic phase was dried over Na₂SO₄, filtered, and concentrated under vacuum to give a residue, which was purified by flash chromatography on silica gel, eluting with ethyl acetate and giving 0.93 g (37%) of 6-bromo-1-methyl-3,4-dihydroisoquinoline as a brownish oil. ¹H-NMR δ (CDCl₃): 7.38 (m, 3H), 3.65 (t, J=7.5 Hz, 2H), 2.67 (t, J=7.5 Hz, 2H), 2.36 ppm (s, 3H).

### Benzoyl isocyanate

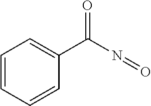

Oxalyl chloride (1.44 mL) was added to a suspension of benzamide (1.21 g, 10 mmol) in DCE under a nitrogen atmosphere. The suspension was stirred at 60° C. for 3 hours, monitoring the progression by NMR, and concentrated in loco under vacuum to give 1.30 g of benzoyl isocyanate (88%) as yellowish oil, with was directly used without any further purification. ¹H-NMR δ (CDCl₃): δ 8.07 (d, J=7.3 Hz, 2H), 7.65 (t, J=7.4 Hz, 1H), 7.49 (m, 2H).

### 9-Bromo-6,7-dihydropyrimido[6,1-a]isoquinoline-2,4-dione (MGX-20)

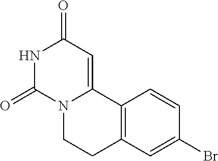 [SMILES: O=c1cc2n(c(=O)[nH]1)CCc1cc(Br)ccc1-2]

TEA (0.758 mL, 5.44 mmol) and benzoyl isocyanate (0.80 g, 5.44 mmol) were added dropwise to a solution of 6-bromo-1-methyl-3,4-dihydroisoquinoline (0.30 g, 1.36 mmol) in toluene. The solution was stirred at 80° C. overnight and monitored by TLC, giving a suspension; at completion, the solid was filtered, washed with toluene and dried under vacuum to give 0.165 g (41%) of 9-bromo-6,7-dihydro-2H-pyrimido[6,1-a]isoquinoline-2,4(3H)-dione as yellowish solid. ¹H-NMR δ (DMSO-d6): δ 11.35 (bs 1H), 7.86 (d, J=6.0 Hz, 1H), 7.65 (s, 1H), 7.54 (d, J=6.0 Hz, 1H), 6.21 (s, 1H), 3.90 (t, J=7.0 Hz, 2H), 2.97 (t, J=7.0 Hz, 2H).

### 9-Bromo-2-chloro-6,7-dihydropyrimido[6,1-a]isoquinolin-4-one (MGX-21)

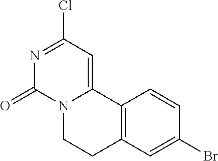 [SMILES: O=c1nc(Cl)cc2n1CCc1cc(Br)ccc1-2]

Compound MGX-21 (1 equivalent) was placed into a 250 mL round bottom flask (RBF) equipped with a stir bar. The compound was subjected to three cycles of vacuum and nitrogen. Added 30 mL POCl₃ into the RBF and stirred at 65° C. for 3 days. The reaction was cooled to room temperature and directly poured into ice cold water (300 mL) inside a 1 L conical flask, which was kept in an ice bath. The pH of this mixture was brought to 7-8 by slowly adding sodium carbonate in the solid form while stirring. This step produced foam/froth. Once pH is reached 7-8, the yellow precipitate was filtered through a frit, washed with cold water (1000 mL) while stirring the precipitate manually with a glass rod. The compound was dried under vacuum.

### 9-Bromo-2-[[(2S)-1,4-dioxan-2-yl]methoxy]-6,7-dihydropyrimido[6,1-a]isoquinolin-4-one (MGX-22)

### 9-Bromo-2-[[(2S)-2,3-dihydro-[1,4]dioxino[2,3-b]pyridin-2-yl]methoxy]-6,7-dihydropyrimido[6,1-a]isoquinolin-4-one (MGX-33)

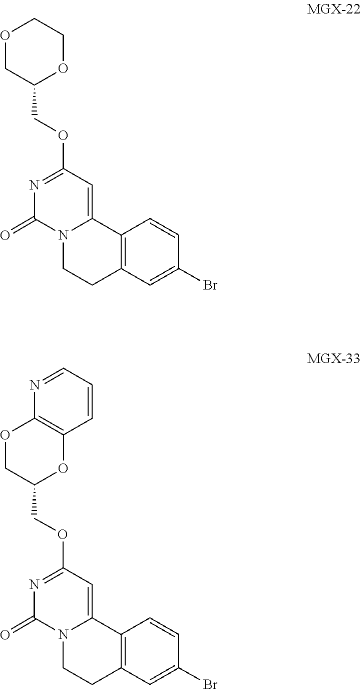 [SMILES: O=c1nc(OC[C@@H]2COCCO2)cc2n1CCc1cc(Br)ccc1-2 ; O=c1nc(OC[C@@H]2COc3ncccc3O2)cc2n1CCc1cc(Br)ccc1-2]

### General Procedure

Compound MGX-21 in a RBF (1 equivalent) was dissolved in dry THF. Added (2R)-[1,4]dioxan-2-yl-methanol or (2R)-hydroxymethyl-2,3-duhydro-[1,4]dioxino[2,3-b]pyridine (1.5 equivalent) and stirred for 5 minutes. Added potassium tert-butoxide (1.5 equivalent) dissolved in dry THE (100 mg/mL concentration) dropwise. The color of the reaction changed to deep red. The reaction was quenched after 60 minutes by adding saturated NH₄Cl (50 mL). Added ethyl acetate (100 mL) and stirred for 5 minutes. The mixture was transferred to a separatory flask and the organic layer was washed with water (twice), brine (once), dried over anhydrous Na₂SO₄, and concentrated in rotavap. Compound was purified via column chromatography using silica gel (20-45 μm) stationary phase and ethyl acetate/methanol gradient (from 1% MeOH to 5% MeOH) mobile phase.

### 9-O-Pinacolatoboro-2-[[(2S)-1,4-dioxan-2-yl]methoxy]-6,7-dihydropyrimido[6,1-a]isoquinolin-4-one (MGX-90)

### 9-O-Pinacolatoboro-2-[[(2S)-2,3-dihydro-[1,4]dioxino[2,3-b]pyridin-2-yl]methoxy]-6,7-dihydropyrimido[6,1-a]isoquinolin-4-one (MGX-100)

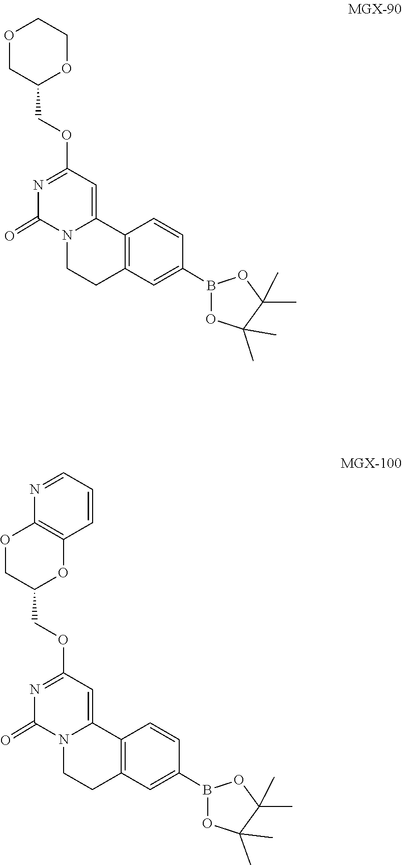 [SMILES: CC1(C)OB(c2ccc3c(c2)CCn2c-3cc(OC[C@@H]3COCCO3)nc2=O)OC1(C)C ; CC1(C)OB(c2ccc3c(c2)CCn2c-3cc(OC[C@@H]3COc4ncccc4O3)nc2=O)OC1(C)C]

### General Procedure

Compound MGX-22 or MGX-33 (1 equivalent) in a sealed tube equipped with a stir bar was dispersed in anhydrous toluene followed by degassing with nitrogen purge for 15 min. Next, bis(pinacolato)diboron (1.5 equivalent) and potassium acetate (3 equivalent) was added to the above tube. This mixture was purged with nitrogen again for another 10 min followed by addition of [Pd)dppf)₂Cl₂](0.2 equivalent). The tube was sealed and stirred at 100° C. for 16 hours. The reaction was allowed to reach room temperature, filtered through a celite pad, and washed with diethyl ether. The solvents were removed in a rotavap and the crude reaction was purified in a silica gel (20-45 m) using ethyl acetate/methanol gradient (from 1% MeOH to 5% MeOH) mobile phase.

MGX-90: ¹H NMR (400 MHz, Chloroform-d) δ 7.82-7.75 (m, 1H), 7.73 (s, 1H), 7.68 (d, J=7.9 Hz, 1H), 6.41 (d, J=1.3 Hz, 1H), 4.50-4.33 (m, 2H), 4.23-4.14 (m, 2H), 3.97 (dddd, J=9.0, 6.3, 3.8, 1.9 Hz, 1H), 3.89-3.60 (m, 5H), 3.48 (dd, J=11.5, 10.1 Hz, 1H), 3.01 (t, J=6.5 Hz, 2H), 1.35 (s, 12H). ¹³C NMR (400 MHz, Chloroform-d) δ 170.73, 156.97, 152.21, 135.14, 134.49, 133.83, 129.43, 124.84, 90.84, 84.33, 74.99, 73.20, 67.79, 66.63, 66.37, 66.06, 40.49, 27.66, 24.84.

MGX-100: ¹H NMR (400 MHz, Chloroform-d) δ 7.88-7.77 (m, 2H), 7.77-7.67 (m, 2H), 7.23 (dd, J=7.8, 1.6 Hz, 1H), 6.89 (dd, J=7.9, 4.8 Hz, 1H), 6.41 (s, 1H), 4.69 (d, J=5.0 Hz, 2H), 4.63-4.50 (m, 2H), 4.33 (dd, J=11.6, 7.2 Hz, 1H), 4.27-4.11 (m, 2H), 3.03 (t, J=6.5 Hz, 2H), 1.36 (s, 12H). ¹³C NMR (400 MHz, Chloroform-d) δ 170.82, 157.18, 153.04, 150.90, 140.48, 138.86, 135.55, 134.91, 134.22, 129.66, 125.43, 125.28, 119.09, 90.89, 84.74, 71.12, 65.93, 64.82, 40.93, 28.01, 25.21.

Synthesis of the HPLC standards MGX-110S and MGX-111S is presented in Scheme 7.

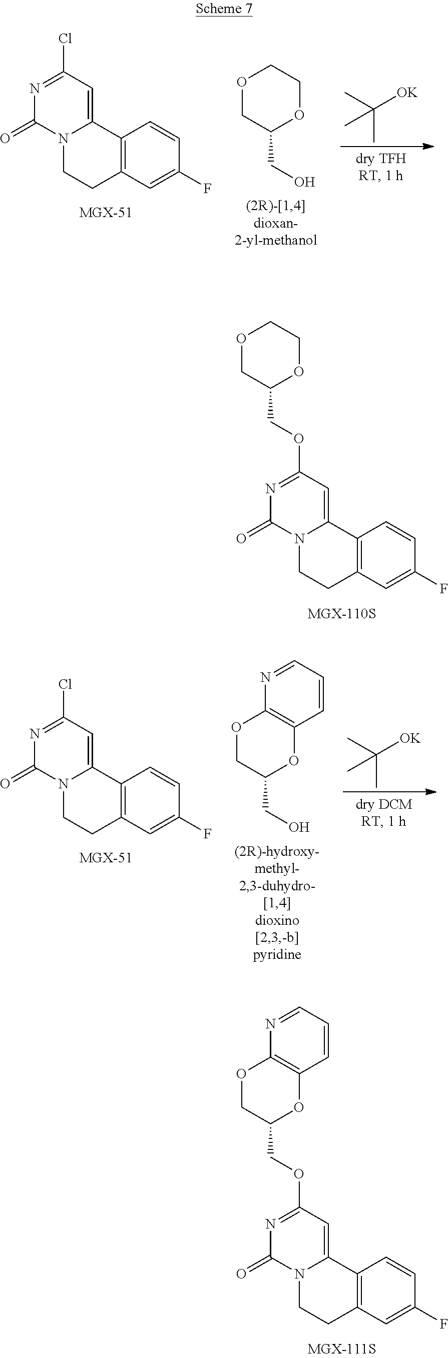

### General Procedure

Compound MGX-51 was charged into a RBF (1 equivalent) and dissolved in dry THF. Added (2R)-[1,4]dioxan-2-yl-methanol or (2R)-hydroxymethyl-2,3-duhydro-[1,4]dioxino[2,3-b]pyridine (1.5 equivalent) and stirred for 5 minutes. Added potassium tert-butoxide (1.5 equivalent) dissolved in dry THF or DCM (100 mg/mL concentration) dropwise. The color of the reaction changed to deep red. The reaction was quenched after 60 minutes by adding saturated NH₄Cl (50 mL). Added ethyl acetate (100 mL) and stirred for 5 minutes. The mixture was transferred to a separatory flask and the organic layer was washed with water (twice), brine (once), dried over anhydrous Na₂SO₄, and concentrated in rotary evaporator. Compound was purified via column chromatography using silica gel (20-45 m) stationary phase and ethyl acetate/methanol gradient (from 1% MeOH to 5% MeOH) mobile phase.

MGX-110S: ¹H NMR (400 MHz, Chloroform-d) δ 7.70 (dd, J=8.7, 5.3 Hz, 1H), 7.15-6.95 (m, 2H), 6.31 (s, 1H), 4.51-4.33 (m, 2H), 4.26-4.15 (m, 2H), 3.97 (dddd, J=10.1, 6.3, 3.8, 2.7 Hz, 1H), 3.90-3.59 (m, 5H), 3.47 (dd, J=11.5, 10.1 Hz, 1H), 3.01 (t, J=6.5 Hz, 2H). ¹³C NMR (400 MHz, Chloroform-d) δ 170.70, 165.91, 163.37, 156.80, 151.50, 139.15, 139.06, 128.34, 128.24, 123.53, 123.50, 115.44, 115.34, 115.22, 115.12, 90.14, 73.19, 67.76, 66.64, 66.37, 66.10, 40.12, 27.92, 27.90. ¹⁹F NMR (400 MHz, Chloroform-d) δ −106.45, −106.46, −106.47, −106.48, −106.49, −106.51.

MGX-111S: ¹H NMR (400 MHz, Chloroform-d) δ 7.91-7.65 (m, 2H), 7.23 (dd, J=7.8, 1.5 Hz, 1H), 7.14-6.97 (m, 2H), 6.94-6.82 (m, 1H), 6.30 (d, J=4.7 Hz, 1H), 4.68 (d, J=5.0 Hz, 2H), 4.62-4.47 (m, 2H), 4.39-4.15 (m, 3H), 3.02 (t, J=6.5 Hz, 2H). ¹³C NMR (400 MHz, Chloroform-d) δ 170.56, 166.16, 163.62, 156.79, 152.10, 150.66, 140.27, 139.36, 139.27, 138.62, 128.59, 128.50, 125.20, 123.54, 118.87, 115.64, 115.55, 115.41, 115.33, 89.98, 70.88, 65.68, 64.60, 40.34, 28.03. ¹⁹F NMR (400 MHz, Chloroform-d) δ −106.08, −106.10, −106.11, −106.12, −106.13.

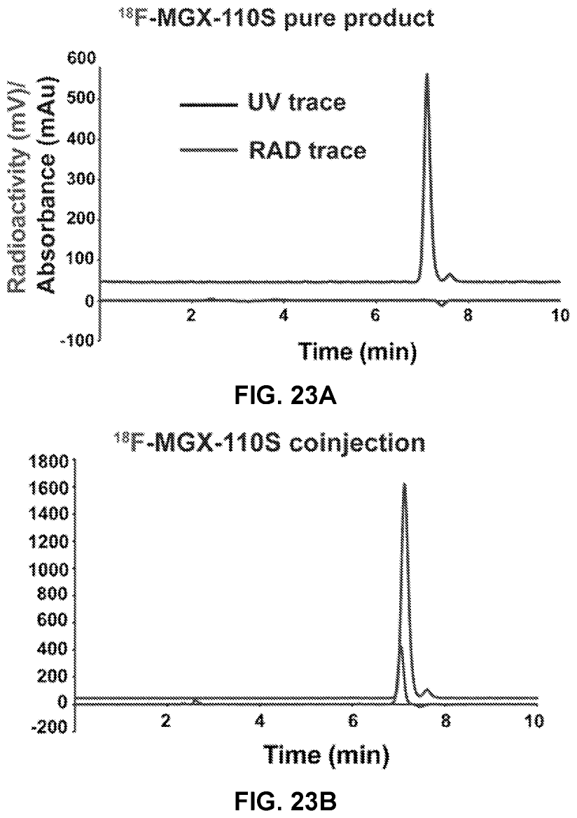

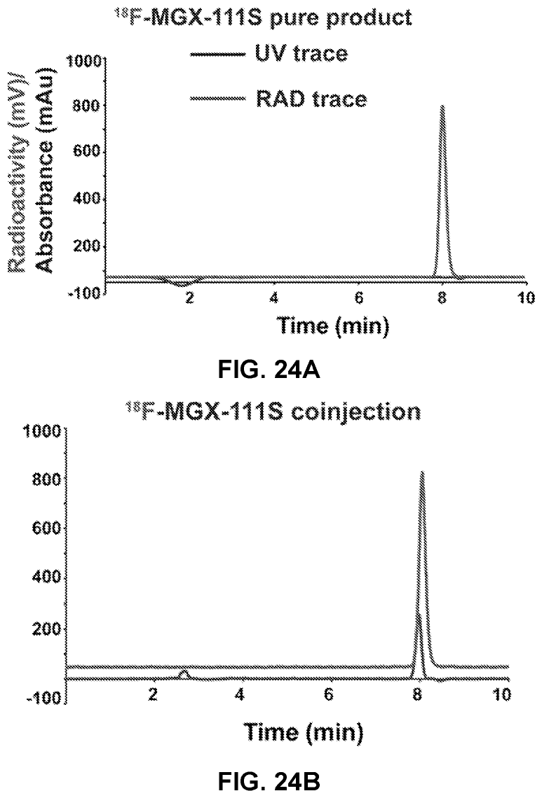

Radiosynthesis of ¹⁸F-MGX-110S and ¹⁸F-MGX-111S is presented in Schemes 8 and 9. HPLC characterization of ¹⁸F-MGX-110S and ¹⁸F-MGX-111S is shown in FIGS. 23A-23B and FIGS. 24A-24B, respectively.

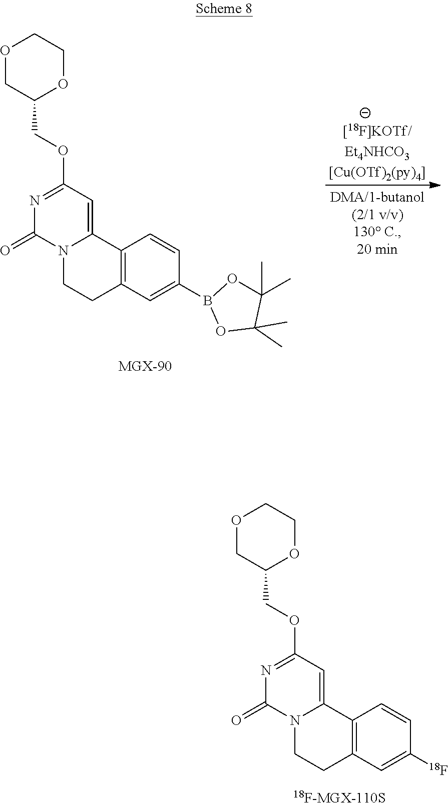 [SMILES: CC1(C)OB(c2ccc3c(c2)CCn2c-3cc(OC[C@@H]3COCCO3)nc2=O)OC1(C)C ; O=c1nc(OC[C@@H]2COCCO2)cc2n1CCc1cc([18F])ccc1-2]

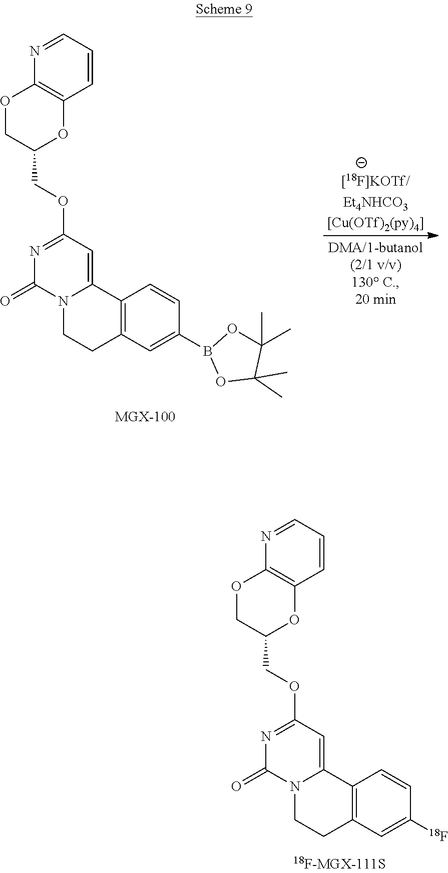 [SMILES: CC1(C)OB(c2ccc3c(c2)CCn2c-3cc(OC[C@@H]3COc4ncccc4O3)nc2=O)OC1(C)C ; O=c1nc(OC[C@@H]2COc3ncccc3O2)cc2n1CCc1cc([18F])ccc1-2]

### General Procedure

The [¹⁸F]fluoride ion was produced using the ¹³O(p,n)¹⁸F nuclear reaction via proton bombardment of a H₂¹⁸O liquid target on a GE PETtrace cyclotron at a beam current of 55 μA for 5 min. The [¹⁸F]fluoride was trapped on a QMA-light Sep-Pak cartridge (Waters) that was preconditioned with ethanol (10 mL), 0.5 M KOTf aqueous solution (10 mL), and MilliQ H₂O (10 mL). Fluoride was eluted into the reaction vessel using a solution of tetraethylammonium bicarbonate (0.05 mg) and KOTf (10 mg) in 0.5 mL of water; the fluoride was then treated with acetonitrile (3 mL) and azeotropically dried at 100° C. first under vacuum and helium overpressure. A solution of MGX-90 or MGX-100 precursor (2.5 mg, 1 equivalent) and Cu(OTf)₂py₄ (1.2 equivalent) in a mixture of DMA (300 μL) and 1-butanol (150 μL) was added, and the reaction mixture was heated at 130° C. for 20 min. After cooling to 50° C., the reaction mixture was diluted with the mobile phase (1.2 mL) and purified by semi-preparative HPLC with mobile phase: water+0.1% TFA/MeCN 0.1% TFA (65:35) as an eluent at a 4 mL/min flow rate. The collected peak was further processed to achieve final formulation of 10% ethanol in saline. Decay uncorrected radiochemical yield=(22.21±11.99) % (n=3), molar activity (Am)=1.85±0.06 Curie/μmol.

Peripheral Blood Mononuclear Cell (PBMC) Isolation & Human Monocyte-Derived Macrophage (hMDM) Differentiation

Deidentified fresh human peripheral blood from Stanford Blood Bank was used to isolate peripheral blood mononuclear cells (PBMCs) via centrifugation and separation by density using Ficoll-Paque Plus (GE Healthcare Life Sciences, Piscataway, NJ, USA). Isolated PBMCs were washed in sterile PBS until a clear supernatant was achieved upon centrifugation. Cells were counted using Trypan blue dye (Life Technologies Cat #15250-061) on an automatic cell counter (Countess II FL). From the clean PBMCs, CD14⁺ monocytes were isolated by negative magnetic selection using the Pan Monocyte Isolation Kit (Miltenyi Biotec Cat #130-096-537) and QuadroMACS™ Starting Kit with LS columns (Miltenyi Biotec Cat #130-091-051). CD14⁺ cells were then resuspended in complete growth medium: RPMI 1640 with Glutamax with 50 ng/mL macrophage colony-stimulating factor (M-CSF, PreproTech Cat #300-25), 1% 100× Antibiotic-Antimycotic (anti-anti, Gibco Cat #15240062), and 10% filtered FBS (Millipore Sigma Cat #12107C), and were plated appropriately (10×10⁶-10×10⁷ cells per T-75 flask in 15 mL complete media). Cells were allowed to incubate at 5% CO₂ and 37° C. for 7-10 days, changing media every 2-3 days.

hMDM Pro-Inflammatory Stimulation Using Lipopolysaccharides (LPS)

CD14⁺ monocytes isolated from PBMCs from human peripheral blood were plated 10×10⁶ cells per 10 cm petri dish in 8-10 mL complete growth medium (for quantitative PCR) or 10⁶-10⁵ cells per well in 1 mL complete growth medium into one 12-well plate per blood donor (radioactive cell binding) and incubated 7-10 days at 5% CO₂ in air at 37° C. These cells were then treated with 100 ng/mL LPS in complete growth medium 24 hr or 72 hr prior to experimentation according to previous working concentrations in the literature.

hMDM mRNA Expression Quantification

The RNeasy Micro Kit (Qiagen #74004) was used to extract ribonucleic acids from cultured hMDM cells, and the amount and purity of the sample was determined by quantifying the RNA/DNA ratio by spectrophotometry (Eppendorf BioSpectrometer). From this sample, complementary DNA (cDNA) was synthesized using the RT² First Strand Kit (Qiagen). Incubation was completed in the Thermal Cycler Mini Amp (Applied Biosystems), according to the kit protocol. Real-time quantitative PCR was then performed in a thermal cycler (the Applied Biosystems QuantStudio 6 Real-Time PCR machine) using SYBER Green Polymerase (Qiagen), cDNA, and primers for βactin (Origene Cat #HP204660), TSPO (Qiagen Cat #PPH02177F-200), and gpr84 (Origine Cat #HK203504). The levels of the housekeeping gene βactin were measured to serve as a normalization for the target Gpr84 and Tspo genes. Each sample was run as three technical replicates. Fold-change for Gpr84 and Tspo was calculated by determining 2^{-ΔΔCt}. Transcripts with undetectable values were assigned a cycle threshold of 37, rather than 40, for analysis. Samples with high variation amongst replicates (SD>0.8) were excluded from analysis.

### HEK Radioactive Cell Binding Assay

GPR84-transduced HEK-293 cells and control HEK-293 cells from Creative Biogene were plated at a concentration of 3×10⁵ cells per well in media (DMEM with 10% FBS and 1% anti-anti) into a 12-well plate for 16-24 hr. HEK-293 cells were stably transduced via the following process from Creative Biogene: construction of pLVX-CMV-Human-GPR84-PGK-Puro lentiviral expression plasmid, lentivirus production, lentivirus transduction, puromycin selection of polyclonal stable HEK293-Human-GPR84 cells, screening of monoclonal stable HEK293-Human-GPR84 cells via limited dilution, validation using qPCR, then mycoplasma detection by PCR and Lonza kit. Once cells reached ˜90% confluency, the radiotracer was prepared. The radiotracer was diluted in DMEM without FBS or antibiotic to yield a 30 mL solution such that the amount of activity was 50 μCi/mL for ¹¹C or 2 μCi/mL for ¹⁸F. Additionally, a blocking solution was prepared consisting of 35 μM GLPG1205 dissolved in DMSO and added to DMEM media+tracer solution. The 12-well plate was incubated for 40 min at 37° C. and 5% CO₂ in air. Following incubation, the cells were washed with PBS, trypsinized, resuspended in DMEM+10% FBS, then pipetted into gamma counting tubes at 500 μL volume. Radioactivity levels bound to cells were measured in a gamma counter (Hidex Oy, Helsinki, Finland), and cell counts were recorded using Trypan blue and an automatic cell counter to normalize radioactivity levels to cell count.

hMDM Radioactive Cell Binding Assay

Human monocyte-derived macrophages (hMDMs) were plated at a concentration of 10⁶-10⁵ cells per well in media (RPMI 1640+glutamax with 10% FBS, 1% anti-anti, and 50 ng/mL M-CSF) into one 12-well plate per blood donor. Once cells adhered to the plate (˜24 hr), 100 ng/mL lipopolysaccharides (LPS) was applied to two of the three rows of the plate. 24 hr later, the [¹¹C]GLPG38, [¹¹C]MGX-10S, or [¹⁸F]MGX-110S radiotracer was prepared. The radiotracer was diluted in RPMI 1640 without FBS or antibiotic to yield a 30 mL solution such that the amount of activity was 50 μCi/mL for ¹¹C or 2 μCi/mL for ¹⁸F. Additionally, a blocking solution was prepared consisting of 35 μM GLPG1205 dissolved in DMSO and added to RPMI 1640 media+tracer solution. The 12-well plates were incubated for 30 min at 3° C. and 5% CO₂ in air. Following incubation, the radioactive media was removed and replaced with 500 μL fresh media, and a 10 min efflux was conducted. After efflux, the media was collected into a gamma counting tube to measure supernatant, and the cells were washed with PBS, trypsinized, resuspended in RPMI 1640+glutamax+10% FBS, then pipetted into gamma counting tubes at 500 μL volume.

Radioactivity levels bound to cells were measured in a gamma counter (Hidex Oy, Helsinki, Finland), and cell counts were recorded using Trypan blue and an automatic cell counter to normalize radioactivity levels to cell count.

### Murine Model of Brain Metastases Study Methods

Six-to-eight-week-old female C57BL/6 mice were injected subcutaneously with 0.25×10⁶ B16F1 melanoma cells on the right flank to generate extracranial tumors. After two days of extracranial tumor inoculation, 5×10³ B16F1 melanoma cells were stereotactically injected into the striatum. Five days after intracranial tumor inoculation, mice were randomized into treatment (n=4) vs no treatment (n=4) groups based on the intracranial bioluminescence signals and monitored daily starting on day 5 for extracranial tumor volume changes. On days 5, 7 and 9 post-intracranial tumor inoculation, mice in the treatment group were treated with anti-PD-1 and anti-CTLA-4 (200 μg via intraperitoneal injection). T2-weighted MRI (Bruker) with a CryoProbe was performed on day 9 after intracranial tumor inoculation to identify brain tumor region of interest. Following MRI, mice underwent PET/CT imaging on the GNEXT PET/CT (Sofie). 100-125 μCi of [¹⁸F]MGX-110S was injected for a 10-min static PET scan followed by CT. Mice were then perfused and organs of interest were dissected and analyzed via gamma counting. Region of interest analysis of the PET images was performed using a 3D volume drawing mode around the intracranial and extracranial tumors (Inveon Research Workspace).

### Experimental Autoimmune Encephalomyelitis (EAE) Imaging Methods

### Animals

All experiments involving animals were completed in accordance with the Stanford Administrative Panel on Laboratory Animal Care (APLAC), which is accredited by the Association for the Assessment and Accreditation of Laboratory Animal Care (AAALAC International). Female C57BL/6 J wild-type mice were purchased from Jackson Laboratories (9 weeks, Jax #00664) and acclimatized for 1 week prior to experiments. Mice were housed in a temperature-controlled environment under a 12-h light/dark schedule with unrestricted access to food/water.

### EAE Induction

10-week old female C57BL/6 mice were induced with EAE using kits from Hooke Laboratories Inc. (cat #EK2110; Lawrence, MA). After anesthetizing mice with isoflurane gas (2.0-3.0% for induction and 1.5-2.5% for maintenance), they were inoculated with myelin oligodendrocyte glycoprotein (MOG35-55) in complete Freund's adjuvant (CFA) (0.2 mL per mouse, S.C.), followed by two pertussis toxin (PTX) injections (110 ng per injection, I.P., 1 and 24 h post-MOG injection). All mice were weighed and scored daily in the morning from day 8 onwards according to standard EAE scoring systems: 0, no paralysis; 1, loss of tail tone; 2, hind limb weakness or paresis; 3, hind limb paralysis; 4, complete hind leg and partial front leg paralysis. EAE mice were given supplemental food and fluids as necessary.

### PET/CT Imaging

On days 15-16 after EAE induction, mice were catheterized and placed on the 4-mouse scanning bed of the Sofie GNEXT PET/CT scanner. Mice were injected with a radiotracer bolus of 130-170 μCi of ¹⁸F-MGX-110S for a 60-min dynamic PET scan followed by CT. Scans were reconstructed from list mode into 19 frames (4×15 s, 4×60 s, 11×300 s) using OSEM3D reconstruction with 3 iterations and 24 subsets. Brain analysis was performed using a semi-automated brain atlas on VivoQuant software (2020 version) and spinal cords were manually drawn as previously described (JoVE).

### Ex Vivo Gamma Counting and Autoradiography

After the PET/CT scan the mice were perfused under 3% isoflurane and organs of interest were dissected out, weighed, and placed on a Hidex gamma counter to quantify the percent of injected dose per gram of tissue. After gamma counting, CNS tissues were frozen and sectioned on a cryotome at 40 μm and placed on a phosphor storage screen overnight. The images were read using a Typhoon imager 18 h after the screen was exposed to the tissues. Additional mice were also injected off of the PET/CT scanner and perfused at 60-min post-injection to increase the N for the ex vivo gamma counting and autoradiography data.

### Results

Binding Affinity of ¹⁸F-MGX-110S and ¹⁸F-MGX-111S

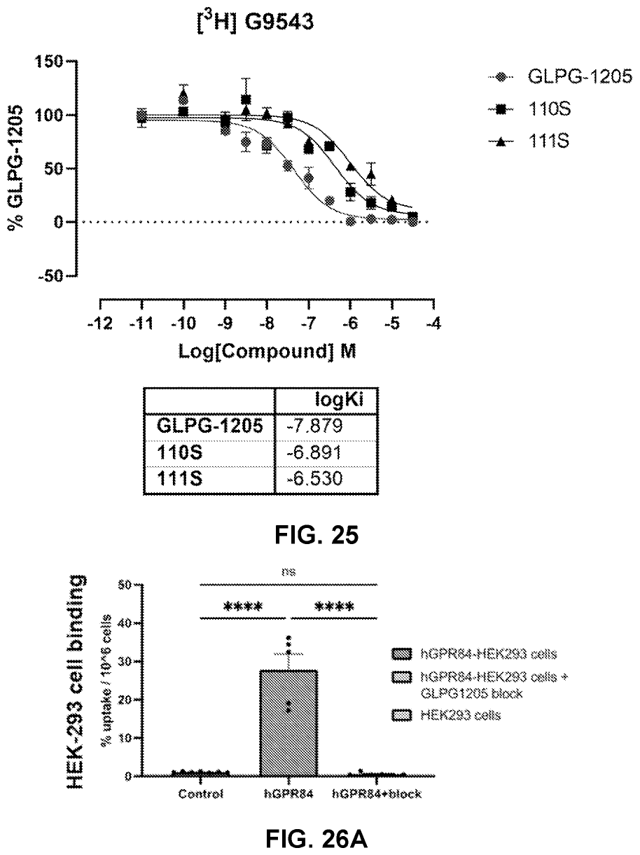

A competition binding experiment. 5 μg of membranes was prepared from a stable cell line expressing hGPR84-Gi fusion protein and measured the ability of the compounds to compete for binding of [³H]G9543 (chemical analog of lead negative allosteric modulator, GLPG1205). Results, including log Kᵢ are shown in FIG. 25.

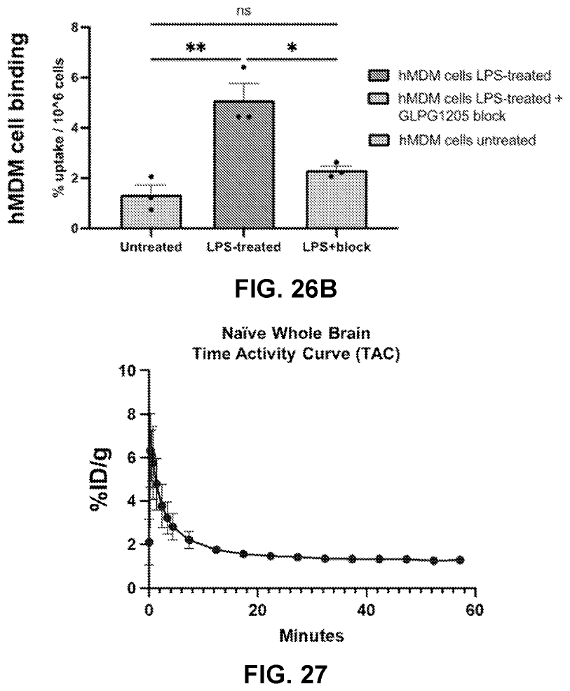

Cell binding experiments were performed first with hGPR84-stably-transduced human embryonic kidney cells (HEK-293) versus control HEK-293 cells (FIG. 26A). To assess binding specificity, cells were incubated with the tracer in absence or presence of a blocking compound, the known GPR84 antagonist (35 μM GLPG1205 in DMSO and media), for 40 mins. Cell binding studies showed 25-fold higher binding of [¹⁸F]MGX-110S to hGPR84-HEK-293 cells vs control cells (p<0.0001, n=5-7). Co-incubation with GPR84 antagonist (GLPG1205, 1000× by mass) reduced tracer binding in hGPR84-HEK293 cells by >90% (p<0.0001, n=5-7) proving high specificity of these tracers in cells. hMDMs challenged with LPS (100 ng/mL) stimulus were found to display significantly higher binding of [¹³F]MGX-110S compared to unstimulated cells (p>0.001, n=3), and this signal was reduced to baseline following co-incubation with GLPG1205 (p>0.001, n=3). Statistical significance was assessed using a one-way ANOVA: *p<0.05, **p<0.01, ****p<0.0001, ns p>0.05 (FIG. 26B).

In mice with no neuroinflammation, exemplary radiotracers are eliminated from the brain in a short period of time. A time activity curve illustrating kinetics of ¹⁸F-MGX-110S in the whole brain of a healthy naïve mouse demonstrating radiotracer crossing the blood brain barrier with over 6% ID/g peak uptake in the first few minutes and a rapid washout with less than 1.5% ID/g at 60-min post-injection is shown in FIG. 27.

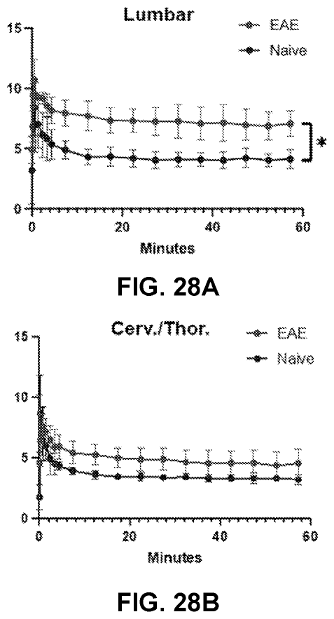

Spinal cord time activity curves illustrating increased signal of ¹³F-MGX-110S in both the lumbar and cervical/thoracic spinal cord were calculated for EAE mice compared to naive mice (FIGS. 28A-28B). Significantly higher signal at 60 min in the lumbar was found with Welch's t test. *p<0.05, **p<0.01, ***p<0.001, ****p<0.0001, ns p>0.05.

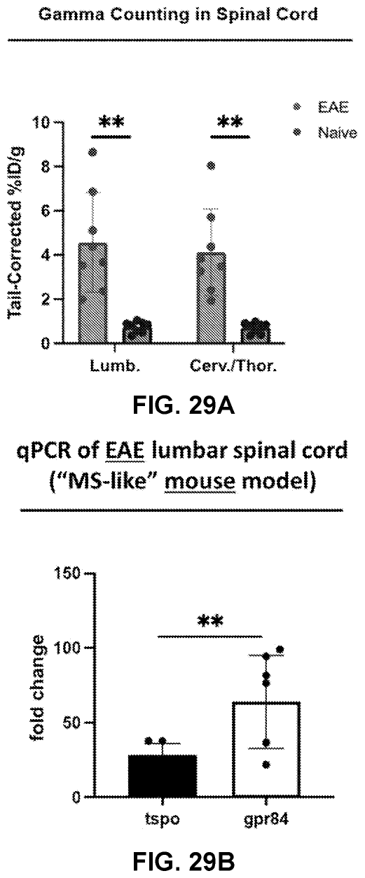

PET/CT imaging was conducted showing elevated signal in the lumbar (white arrow), thoracic (green arrow) and cervical (orange arrow) spinal cord regions, brain stem (blue arrow), and adipose tissue of EAE mouse compared to negligible signal in naive mouse. Both naive and EAE mice show signal in the liver and gut, indicative of tracer metabolism and clearance. FIG. 29A shows gamma counting of spinal cords confirmed elevated uptake of ¹⁸F-MGX-110S using Welch's t test. *p<0.05, **p<0.01, ***p<0.001, ****p<0.0001, ns p>0.05. qPCR was performed, showing that GPR84 is significantly upregulated in the spinal cord of an MS mouse model and this elevation can be detected using the disclosed tracers with a 1.5-1.9 fold increase in signal (FIG. 29B).

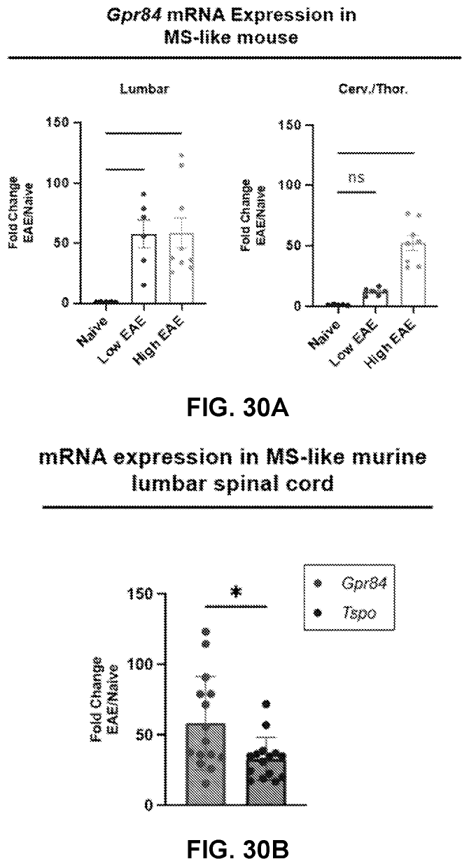

mRNA expression of Gpr84 in spinal cords from EAE mice versus naive mice demonstrating was evaluated, showing significantly higher Gpr84 expression in all segments of the spinal cord via ordinary one way ANOVA test (FIG. 30A). FIG. 30B shows a comparison using Welch's t test of naïve normalized Gpr84 vs Tspo mRNA in EAE spinal cord reveals significantly higher Gpr84 expression, indicating higher sensitivity of this biomarker in EAE mice. *p<0.05, **p<0.01, ***p<0.001, ****p<0.0001, ns p>0.05.

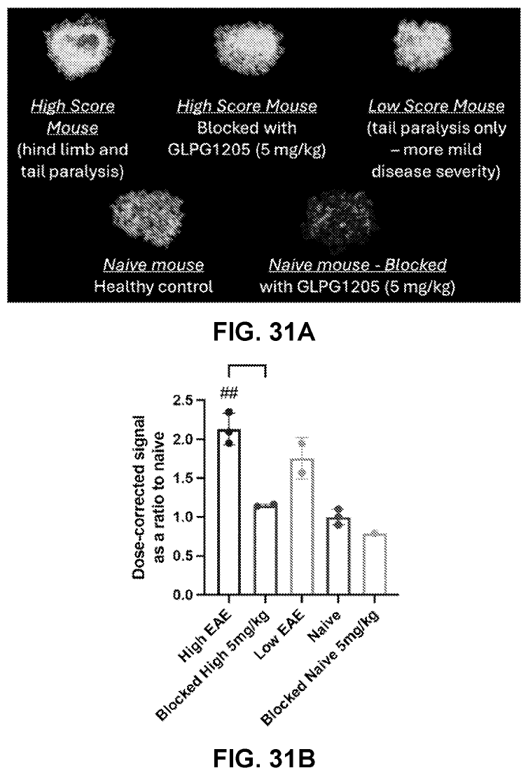

Representative high-resolution images of tracer binding in ex vivo autoradiography of lumbar spinal cord sections (40 μm-thick) are seen in FIG. 31A, showing tracer binding is highly specific for GPR84. FIG. 31B shows quantification of autoradiography data, which demonstrates significantly more tracer binding in the high score EAE mouse compared to both the blocked high score and naïve groups using Welch's t test. #denotes comparison to naïve. *^{, #}p<0.05, **^{, ##}p<0.01, ***^{, ###}p<0.001, ****^{, ####}p<0.0001, ns p>0.05.

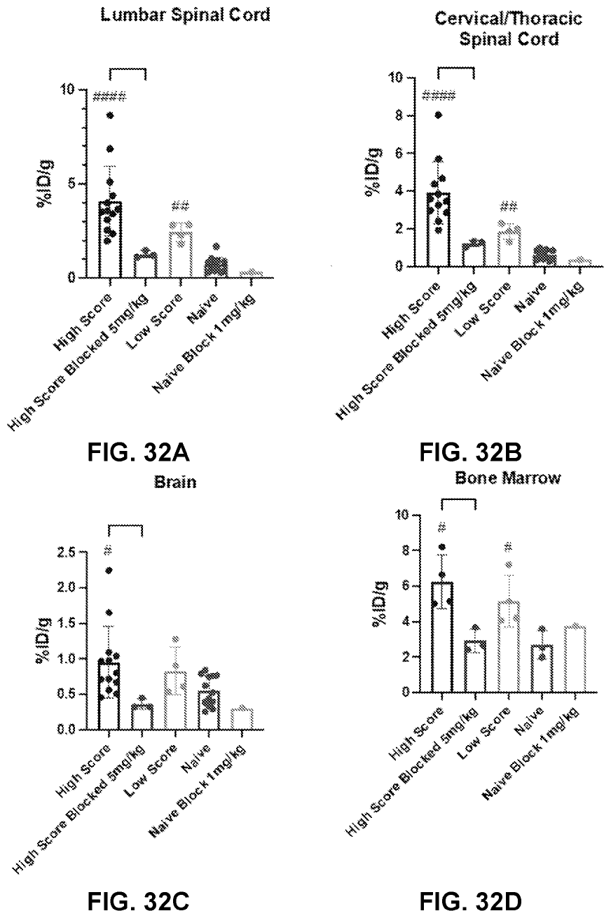

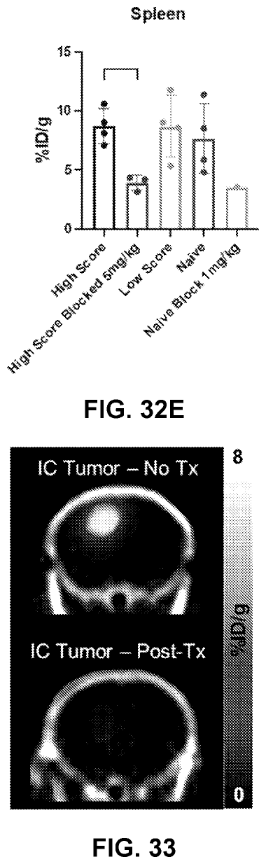

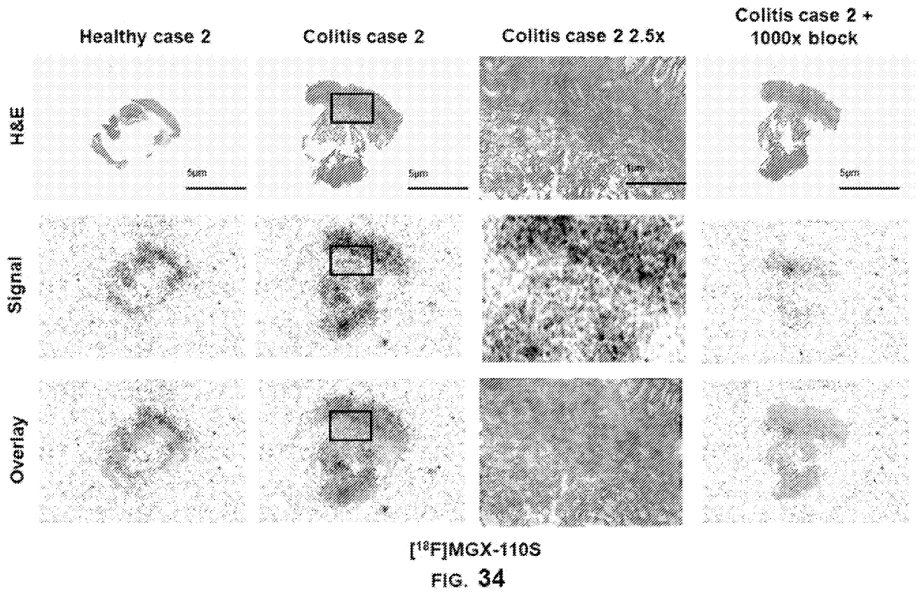

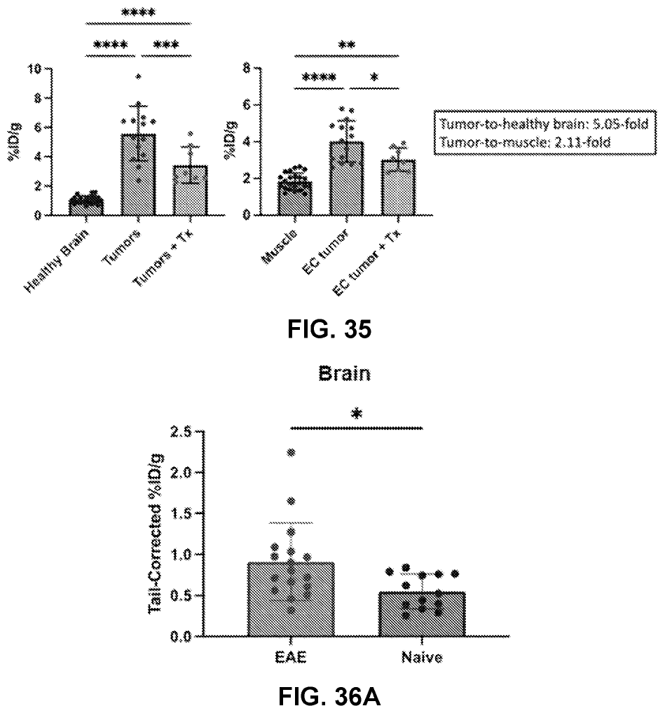

Post-scan ex vivo gamma counting confirms specificity of ¹⁸F-MGX-110S in all organs via significant blocking with GLPG1205, a known GPR84 agonist (FIGS. 32A-32E). Additionally, gamma counting shows significantly higher binding in high scoring EAE mice compared to Naïve mice in all organs except spleen and significantly higher radiotracer binding in the spinal cords of the low scoring EAE mice using Welch's t test. #denotes comparison to naïve. *^{,#}p<0.05, **^{,##}p<0.01, ***^{,###}p<0.001, ****^{,####}p<0.0001, ns p>0.05.

¹³F-MGX-110S can be used to monitor response to immunotherapy in a mouse model of brain metastasis. Six-to-eight-week-old female C57BL/6 mice were implanted subcutaneously with 0.25×10⁶ B16F1 melanoma cells on the right flank and with 5×103 B16F1 melanoma cells in the striatum two days post-extracranial tumor inoculation. Mice were randomized into treatment (n=4) and no treatment (n=4) groups on day 5 post-intracranial tumor inoculation based on the intracranial bioluminescence signals. Mice in the treatment group were treated with anti-PD-1 and anti-CTLA-4 (200 μg via intraperitoneal injection) on days 5, 7 and 9. MRI was performed on day 9 post-intracranial tumor implantation to identify brain tumor of interest, and PET/CT imaging using ¹⁸F-MGX-110S followed. Representative PET/CT images of mouse with brain metastasis showed significantly elevated signal in the intracranial tumor compared to immunotherapy-treated mouse (FIG. 33).

### Multiple Sclerosis Model

Multiple sclerosis (MS) is a demyelinating, neuroinflammatory disease that causes substantial morbidity and diminished quality of life typically affecting young adults. Myeloid cells are fundamental to the progression and remission of MS; activated macrophages and microglia are the predominant immune cells associated with acute and chronic-active central nervous system (CNS) lesions. Unfortunately, existing imaging strategies (e.g., positron emission tomography [PET] using translocator protein 18 kDa [TSPO]-targeted tracers) for detecting myeloid cells lack specificity and cannot distinguish between beneficial and harmful immune responses. GPR84 is an immune-metabolic receptor that is significantly induced on myeloid cells specifically under pro-inflammatory conditions. Herein is reported the synthesis of a new PET tracer for GPR84, ¹⁸F-MGX-110S. Subsequently, this tracer is evaluated vs. ¹⁸F-GE180 (TSPO-PET tracer) in a mouse model of MS (experimental autoimmune encephalomyelitis; EAE) to investigate its ability to detect spinal cord neuroinflammation known to occur in this model.

Radiosynthesis of ¹⁸F-MGX-110S was achieved by copper-mediated fluorination of a Bpin precursor in a mixture of dimethylacetamide at 130° C. for 20 min. C57BL/6 mice were induced with EAE using myelin oligodendrite glycoprotein (MOG₃₅₋₅₅) emulsified in complete Freund's adjuvant and weighed/scored daily for disease severity. Expression of Gpr84 and Tspo was assessed in EAE (n=10-16 mice of low and high disease severities) versus naïve mice (n=5) using quantitative polymerase chain reaction (qPCR) of CNS tissues. In vivo kinetics, spatial distribution, and BBB permeability of ¹⁸F-MGX-110S was assessed via 60-min dynamic PET/CT imaging of naïve C57BL/6 (n=3) and high scoring EAE mice (n=3). Ex vivo gamma counting of tissues of interest was conducted using naïve (n=8) and EAE (n=8) mice 60-min post-tracer injection. Lastly, EAE (n=3) and naïve (n=4) mice were injected with ¹⁸F-GE180 and dissected 60 min later for gamma counting as a comparison.

¹⁸F-MGX-110S was successfully synthesized (retention time [tR]=4.91 min), confirmed by co-injection of the tracer with ¹⁹F-MGX-110S (tR=4.82 min), with radiochemical purity >99%, radiochemical conversion=54.40±30.38%, and decay-uncorrected radiochemical yield=22.21±11.99%. qPCR revealed significantly elevated Gpr84 expression in high scoring EAE spinal cords and brain vs. naïve mice (all p<0.05) and vs. low score EAE lumbar spinal cords and brain (p=0.0113, p=0.0205, respectively). qPCR also demonstrated significantly higher (p=0.0147) Gpr84 expression in EAE lumbar spinal cord vs. Tspo indicating greater sensitivity of GRP84 as a biomarker in this model. Whole body PET/CT images of EAE mice showed elevated signal in the spinal cord, brain stem, and adipose tissue vs. naive mice. Whole brain TACs for naïve mice (n=3) revealed rapid entry of ¹⁸F-MGX-110S into the brain with a peak uptake of 6.33±1.70% ID/g at 0.25 min and rapid washout to 1.28±0.10% ID/g by 60 min. TACs for EAE vs. naïve mice illustrated increased signal in cervical/thoracic spinal cord (4.54±1.16 vs. 3.21±0.42% ID/g at 60 min) and significantly higher signal in lumbar spinal cord (p=0.0206 at 60 min; 7.08±1.05 vs. 4.13±0.79% ID/g). Ex vivo gamma counting confirmed significantly elevated tracer binding in both the cervical/thoracic and lumbar spinal cord (both p<0.01), but not in whole brain. Crucially, the signal-to-background ratio (tracer binding in EAE-to-naive) for ¹⁸F-MGX-110S in lumbar spinal cord was found to be 2.5-fold higher than ¹⁸F-GE180.

We have successfully made a novel ¹⁸F-labeled GPR84 PET tracer and shown it can detect neuroinflammation in EAE mice spinal cords with greater sensitivity than a TSPO-PET tracer.

### ¹⁸F-MGX-111S

Radiosynthesis of ¹⁸F-MGX-111S involved the reaction between Bpin precursor (1 equivalent) and Cu(OTf)₂py₄ (1.2 equivalent) in a mixture of dimethylacetamide (300 μL) and 1-butanol (150 μL) which was heated at 130° C. for 20 min. The reaction mixture was quenched with mobile phase (1.5 mL) and purified by semi-preparative HPLC with mobile phase: water+0.1% TFA/MeCN 0.1% TFA (65:35) as an eluent at a 4 mL/min flow rate. The collected peak was further processed to achieve final formulation of 10% ethanol in saline. HPLC chromatograms. Pure ¹⁸F-MGX-111S (HPLC retention time=8.06 min, FIG. 24A) was confirmed by analytical HPLC using co-injection of the tracer with cold ¹⁹F-MGX-110S compound (HPLC retention time=7.94 min, FIG. 24B).

### Example 5: ¹⁸F Fluorosulfonates

### Synthesis

Non-radioactive standards for MGX-113 and MGX-125 were synthesized according to the procedures shown in Scheme 10 and Scheme 11, respectively.

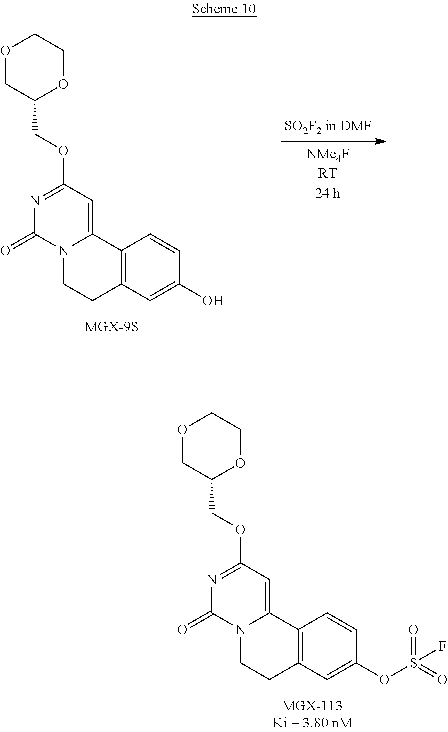 [SMILES: O=c1nc(OC[C@@H]2COCCO2)cc2n1CCc1cc(O)ccc1-2 ; O=c1nc(OC[C@@H]2COCCO2)cc2n1CCc1cc(OS(=O)(=O)F)ccc1-2]

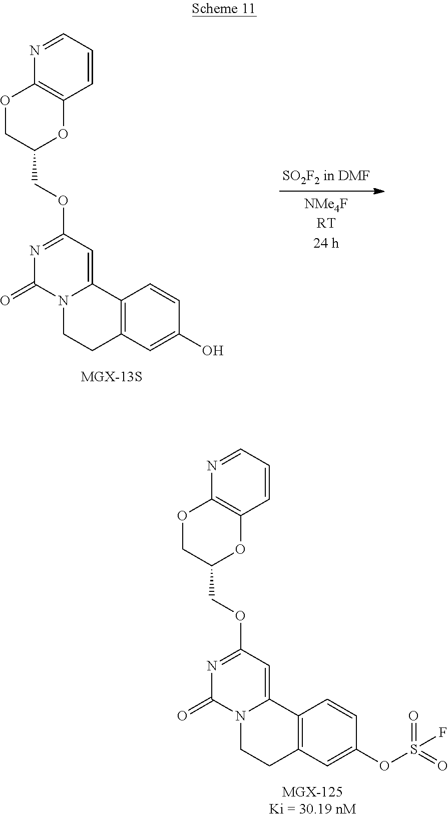 [SMILES: O=c1nc(OC[C@@H]2COc3ncccc3O2)cc2n1CCc1cc(O)ccc1-2 ; O=c1nc(OC[C@@H]2COc3ncccc3O2)cc2n1CCc1cc(OS(=O)(=O)F)ccc1-2]

Radioactive compounds ¹⁸F-MGX-113 and ¹⁸F-MGX-125 were synthesized according to the procedures shown in Scheme 12 and Scheme 13, respectively.

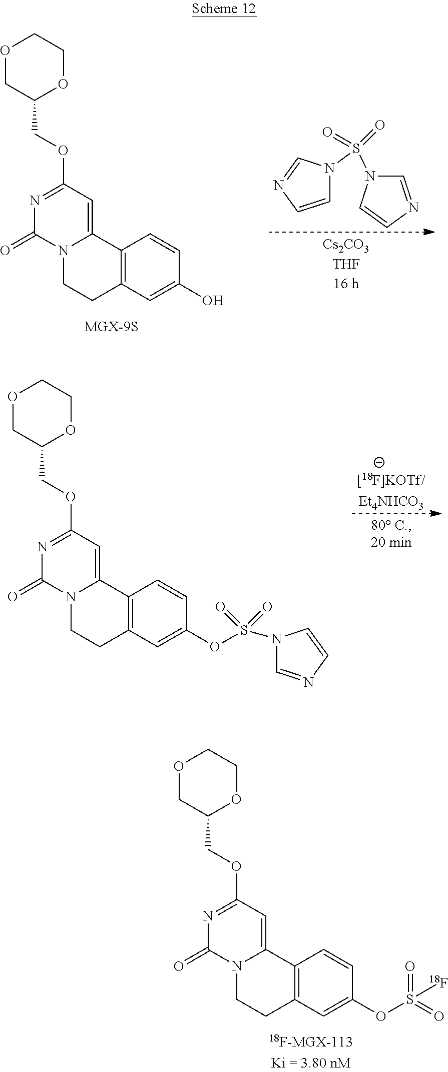 [SMILES: O=c1nc(OC[C@@H]2COCCO2)cc2n1CCc1cc(O)ccc1-2 ; O=S(=O)(n1ccnc1)n1ccnc1 ; O=c1nc(OC[C@@H]2COCCO2)cc2n1CCc1cc(OS(=O)(=O)n3ccnc3)ccc1-2 ; O=c1nc(OC[C@@H]2COCCO2)cc2n1CCc1cc(OS(=O)(=O)[18F])ccc1-2]

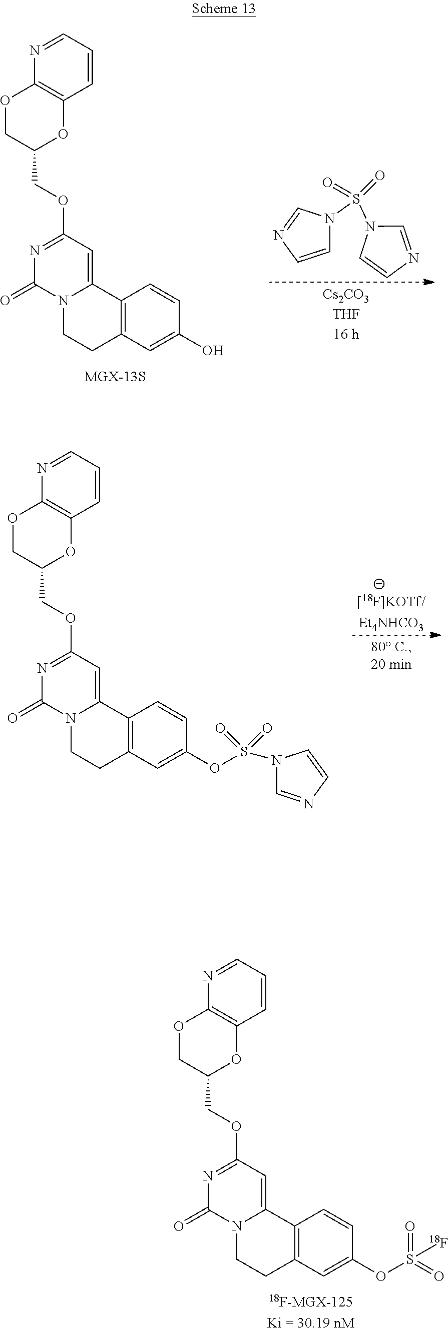 [SMILES: O=c1nc(OC[C@@H]2COc3ncccc3O2)cc2n1CCc1cc(O)ccc1-2 ; O=S(=O)(n1ccnc1)n1ccnc1 ; O=c1nc(OC[C@@H]2COc3ncccc3O2)cc2n1CCc1cc(OS(=O)(=O)n3ccnc3)ccc1-2 ; O=c1nc(OC[C@@H]2COc3ncccc3O2)cc2n1CCc1cc(OS(=O)(=O)[18F])ccc1-2]

### Characterization of MGX-113 and MGX-125

Binding affinity studies were conducted for MGX-113 and MGX-125 controls using a competition binding assay using membrane from Flp-In TREx 293 cells expressing the human GPR84-Gαᵢ₂ fusion protein (n=3).

A cAMP functional assay was conducted for MGX-113 and MGX-125 controls using Flp-In TREx 293 cells expressing the human GPR84-Gαᵢ₂ fusion protein.

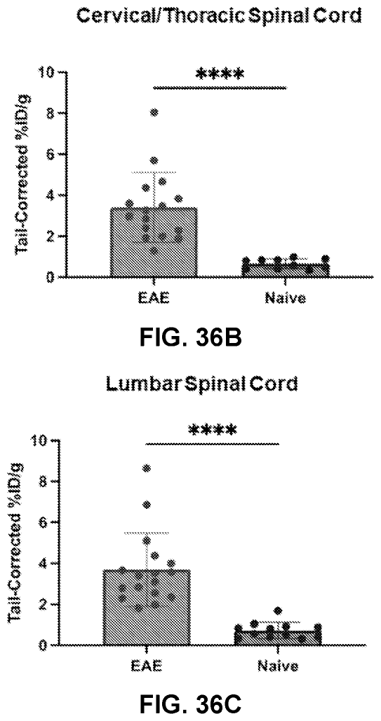

Post PET scan perfusion and dissection of CNS tissue followed by gamma counting confirms significantly decreased radiotracer binding in all CNS organs in a mouse model in brain (FIG. 36A), cervical/thoracic spinal cord (FIG. 36B), and lumbar spinal cord (FIG. 36C) tissues.

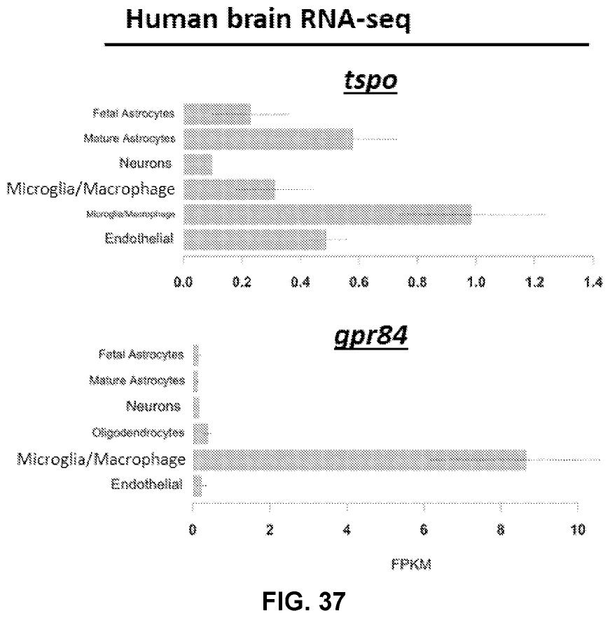

It has further been shown using human brain RNA-seq, mouse brain analysis, and cellular analysis in human monocyte derived macrophages that GPR84 is a more sensitive and specific biomarker of microglia/macrophages than TSPO (see FIG. 37).

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Absinta, M.; et al. A Lymphocyte-Microglia-Astrocyte Axis in Chronic Active Multiple Sclerosis. Nature 2021, 597, 709-714.
2. Alexoff, D. L.; et al. Reproducibility of 11C-Raclopride Binding in the Rat Brain Measured with the MicroPET R₄: Effects of Scatter Correction and Tracer Specific Activity. J. Nucl. Med. 2003, 44, 815-822.
3. Altomonte, S.; et al. Discovery of a High-Affinity Fluoromethyl Analog of [11C]5-Cyano-N-(4-(4-Methylpiperazin-1-YI)-2-(Piperidin-1-YI)Phenyl)Furan-2-Carboxamide ([11C]CPPC) and Their Comparison in Mouse and Monkey as Colony-Stimulating Factor 1 Receptor Positron Emission Tomography Radioligands. ACS Pharmacol. Transl. Sci. 2023, 6, 614-632.
4. Audoy-Rémus, J.; et al. GPR84 Deficiency Reduces Microgliosis, but Accelerates Dendritic Degeneration and Cognitive Decline in a Mouse Model of Alzheimer's Disease. Brain Behav. Immun. 2015, 46, 112-120.
5. Bertoglio, D.; et al. Validation and Noninvasive Kinetic Modeling of [11C]UCB-J PET Imaging in Mice. J. Cereb. Blood Flow Metab. 2020, 40, 1351-1362.
6. Betlazar, C.; et al. Cellular Sources and Regional Variations in the Expression of the Neuroinflammatory Marker Translocator Protein (TSPO) in the Normal Brain. Int. J. Mol. Sci. 2018, 19 (9), 2707.
7. Bolchi, C.; et al. Univocal Syntheses of 2- and 3-Hydroxymethyl-2,3-Dihydro[1,4]Dioxino[2,3-b]Pyridine Enantiomers. Tetrahedron: Asymmetry 2005, 16, 3380-3384.
8. Bouchard, C.; et al. G Protein-Coupled Receptor 84, a Microglia-Associated Protein Expressed in Neuroinflammatory Conditions. Glia 2007, 55, 790-800.
9. Forsman, H.; et al. Function and Regulation of GPR84 in Human Neutrophils. Br. J. Pharmacol. 2023, DOI: 10.1111/bph.16066.
10. Hamesch, K.; et al. Lipopolysaccharide-Induced Inflammatory Liver Injury in Mice. Lab. Anim. 2015, 49, 37-46.
11. Hopperton, K. E.; et al. Markers of Microglia in Post-Mortem Brain Samples from Patients with Alzheimer's Disease: A Systematic Review. Mol. Psychiatry 2018, 23, 177-198.
12. Horti, A. G.; et al. PET Imaging of Microglia by Targeting Macrophage Colony-Stimulating Factor 1 Receptor (CSF1R). Proc. Natl. Acad. Sci. U.S.A 2019, 116, 1686-1691.
13. International Multiple Sclerosis Genetics Consortium. Multiple Sclerosis Genomic Map Implicates Peripheral Immune Cells and Microglia in Susceptibility. Science 2019, 365 (6460), No. eaav7188.
14. James, M. L.; et al. Synthesis and in Vivo Evaluation of a Novel Peripheral Benzodiazepine Receptor PET Radioligand. Bioorg. Med. Chem. 2005, 13, 6188-6194.
15. Jenkins, L.; et al. Discovery and Characterization of Novel Antagonists of the Proinflammatory Orphan Receptor GPR84. ACS Pharmacol. Transl. Sci. 2021, 4, 1598-1613.
16. Labeguere, F.; et al. Discovery of 9-Cyclopropylethynyl-2-((S)-1-[1,4]-Dioxan-2-Ylmethoxy)-6,7-Dihydropyrimido[6,1-a]Isoquinolin-4-One (GLPG1205), a Unique GPR84 Negative Allosteric Modulator Undergoing Evaluation in a Phase II Clinical Trial. J. Med. Chem. 2020, 63, 13526-13545.
17. Li, Y.; et al. Fluorine-18-Labeled Diaryl-Azines as Improved β-Amyloid Imaging Tracers: From Bench to First-in-Human Studies. J. Med. Chem. 2023, 66, 4603-4616.
18. Madeddu, S.; et al. Identification of Glial Activation Markers by Comparison of Transcriptome Changes between Astrocytes and Microglia Following Innate Immune Stimulation. PLoS One 2015, 10, No. e0127336.
19. Mahmud, Z. A.; et al. Three Classes of Ligands Each Bind to Distinct Sites on the Orphan G Protein-Coupled Receptor GPR84. Sci. Rep. 2017, 7, 17953.
20. Marsango, S.; et al. Regulation of the Pro-Inflammatory G Protein-Coupled Receptor GPR84. Br. J. Pharmacol. 2023, DOI: 10.1111/bph.16098.
21. Mrdjen, D.; et al. High-Dimensional Single-Cell Mapping of Central Nervous System Immune Cells Reveals Distinct Myeloid Subsets in Health, Aging, and Disease. Immunity 2018, 48, 380-395.e6.
22. Nicol, L. S. C.; et al. The Role of G-Protein Receptor 84 in Experimental Neuropathic Pain. J. Neurosci. 2015, 35, 8959-8969.
23. Nutma, E.; et al. Cellular Sources of TSPO Expression in Healthy and Diseased Brain. Eur. J. Nucl. Med. Mol. Imaging 2021, 49, 146-163.
24. Prater, K. E.; et al. Human Microglia Show Unique Transcriptional Changes in Alzheimer's Disease. Nat. Aging 2023, 3, 894-907.
25. Qin, L.; et al. Systemic LPS Causes Chronic Neuroinflammation and Progressive Neurodegeneration. Glia 2007, 55, 453-462.
26. Recio, C.; et al. Activation of the Immune-Metabolic Receptor GPR84 Enhances Inflammation and Phagocytosis in Macrophages. Front. Immunol. 2018, 9, 1419.
27. Saniere, L.; et al. Characterization of GLPG1205 in Mouse Fibrosis Models: A Potent and Selective Antagonist of GPR84 for Treatment of Idiopathic Pulmonary Fibrosis. In A19. Less Idiopathic: Structural and Functional Abnormalities in IPF; American Thoracic Society: 2019; pp A1046-A1046.
28. Shi, K.; et al. Global Brain Inflammation in Stroke. Lancet Neurol. 2019, 18, 1058-1066.
29. Shrum, B.; et al. A Robust Scoring System to Evaluate Sepsis Severity in an Animal Model. BMC Res. Notes 2014, 7, 233.
30. Steen, E J L et al. Front Nucl Med, 2022. 2:853475.
31. Takamura, Y.; et al. In Vivo Receptor Visualization and Evaluation of Receptor Occupancy with Positron Emission Tomography. J. Med. Chem. 2021, 64, 5226-5251.
32. Territo, P. R.; et al. Characterization of 11C-GSK1482160 for Targeting the P2×7 Receptor as a Biomarker for Neuroinflammation. J. Nucl. Med. 2017, 58, 458-465.
33. Tewari, M.; et al. Emerging Role of P2×7 Receptors in CNS Health and Disease. Ageing Res. Rev. 2015, 24, 328-342.
34. Timmis, H.; et al. GLPG1205, a GPR84 Modulator: Safety, Pharmacokinetics, and Pharmacodynamics in Healthy Subjects. Clin. Pharmacol. Drug Dev. 2021, 10, 994-1006.
35. Van de Bittner, G. C.; et al. A Philosophy for CNS Radiotracer Design. Acc. Chem. Res. 2014, 47, 3127-3134.
36. Vivash, L.; et al. Imaging Microglial Activation with TSPO PET: Lighting Up Neurologic Diseases? J. Nucl. Med. 2016, 57, 165-168.
37. Wager, T. T.; et al. Central Nervous System Multiparameter Optimization Desirability: Application in Drug Discovery. ACS Chem. Neurosci. 2016, 7, 767-775.
38. Wager, T. T.; et al. Moving beyond Rules: The Development of a Central Nervous System Multiparameter Optimization (CNS MPO) Approach to Enable Alignment of Druglike Properties. ACS Chem. Neurosci. 2010, 1, 435-449.
39. Witney, T. H.; et al. PET Imaging of Tumor Glycolysis Downstream of Hexokinase through Noninvasive Measurement of Pyruvate Kinase M2. Sci. Transl. Med. 2015, 7, 310ra169.
40. Yang, C. et al., Exp. Brain Res. 2021, 239:3601-3613.

1. A radiolabeled compound having a structure of Formula I: 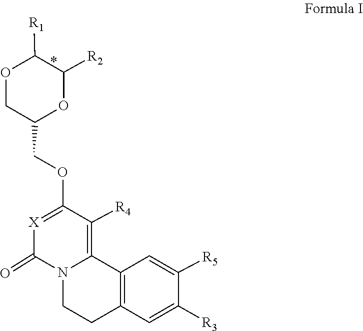
wherein X is N or CH;
wherein R₁ and R₂ are both hydrogen and the bond marked by * is a single bond, or wherein R₁ and R₂ are bridged to form a heteroaromatic ring and the bond marked by * is a component of the heteroaromatic ring;
wherein R₃ comprises a halogen, C1-C4 alkyl ether, or fluorosulfonate group;
wherein R₃ comprises at least one radioisotope; and
wherein R₄ and R₅ are independently hydrogen or methyl.

2. The radiolabeled compound of claim 1, wherein the at least one radioisotope is selected from ¹¹C and ¹⁸F.

3. The radiolabeled compound of claim 1, wherein X is N.

4. The radiolabeled compound of claim 1, wherein X is CH.

5. The radiolabeled compound of claim 1, wherein R₁ and R₂ are both hydrogen and the bond marked by * is a single bond.

6. The radiolabeled compound of claim 1, wherein the radiolabeled compound has the structure

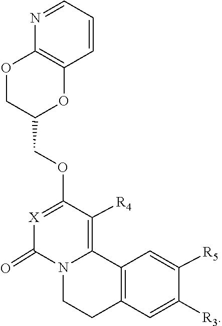

7.-10. (canceled)

11. The radiolabeled compound of claim 1, wherein R₃ is selected from

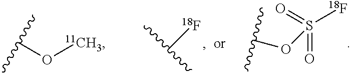

12. The radiolabeled compound of claim 1, wherein the radiolabeled compound is selected from

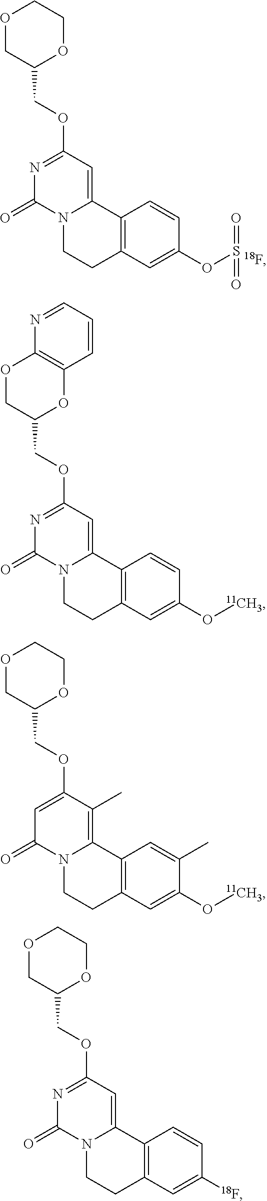

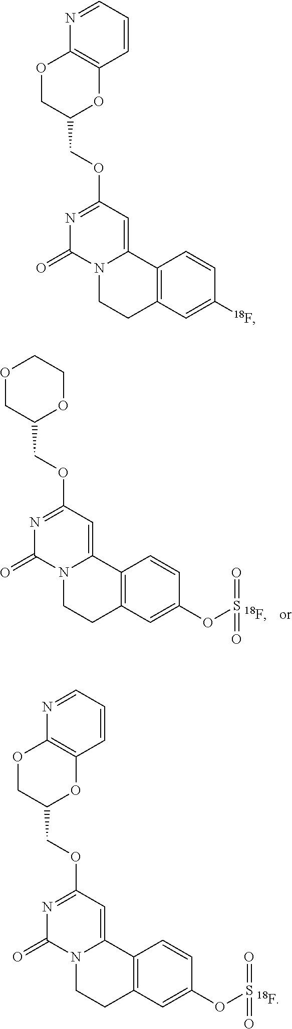

13. The radiolabeled compound of claim 1, wherein the radiolabeled compound undergoes beta plus decay and emits a positron.

14.-15. (canceled)

16. The radiolabeled compound of claim 1, wherein the radiolabeled compound is capable of penetrating an intact blood-brain barrier (BBB).

17. A pharmaceutical composition comprising the radiolabeled compound of claim 1.

18. (canceled)

19. A method for imaging inflammation in a subject, the method comprising:
(a) administering the radiolabeled compound of claim 1 to the subject; and
(b) performing positron emission tomography (PET) on the subject.

20. The method of claim 19, wherein the inflammation is associated with increased GPR84 expression relative to a level of GPR84 expression in a subject not experiencing inflammation.

21. The method of claim 1, wherein the inflammation comprises neuroinflammation, gut inflammation, or any combination thereof.

22. The method of claim 19, wherein the radiolabeled compound binds to GPR84.

23. The method of claim 19, wherein the subject is a mammal.

24. (canceled)

25. A method for determining a stage of a disease associated with increased GPR84 expression in a subject, the method comprising:
(a) administering the radiolabeled compound of claim 1 to the subject;
(b) performing a positron emission tomography (PET) scan on the subject; and
(c) comparing results of the PET scan to one or more standard PET scans.

26. (canceled)

27. The method of claim 25, wherein the disease comprises Alzheimer's disease, multiple sclerosis, glioblastoma, brain metastases, stroke, central nervous system (CNS) injury, a psychiatric disorder, multiple sclerosis, ulcerative colitis, another inflammatory bowel disease, atherosclerosis, pulmonary fibrosis, arthritis, neuropathic pain or other types of pain, chronic fatigue syndrome, fibromyalgia, a bacterial or viral infection, cancer, or any combination thereof.

28. A method of monitoring immune response in a subject in response to a treatment for a disease associated with increased GPR84 expression, the method comprising:
(a) administering the radiolabeled compound of claim 1 to the subject;
(b) performing a positron emission tomography (PET) scan on the subject; and
(c) comparing results of the PET scan to results of an initial PET scan of the subject, wherein the initial PET scan was conducted prior to disease treatment or at an earlier stage of disease treatment.

29. (canceled)

30. The method of claim 28, wherein the disease comprises Alzheimer's disease, multiple sclerosis, glioblastoma, brain metastases, stroke, central nervous system (CNS) injury, a psychiatric disorder, multiple sclerosis, ulcerative colitis, another inflammatory bowel disease, atherosclerosis, pulmonary fibrosis, arthritis, neuropathic pain or other types of pain, chronic fatigue syndrome, fibromyalgia, a bacterial or viral infection, cancer, or any combination thereof.
